SEQUENCE LISTING (57 sequences)

SEQ ID NO: 1 - protein, 9 aa, synthetic construct
  REGION 1..9: note = Description of sequence: E7 epitope component
YMLDLQPET

SEQ ID NO: 2 - protein, 98 aa, synthetic construct
  REGION 1..98: note = Description of sequence: E7 epitope component
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 3 - protein, 8 aa, Homo sapiens
  REGION 1..8: note = Description of sequence: VH CDR1
GFTFSDYY

SEQ ID NO: 4 - protein, 8 aa, Homo sapiens
  REGION 1..8: note = Description of sequence: VH CDR2
IYGGGGST

SEQ ID NO: 5 - protein, 16 aa, Homo sapiens
  REGION 1..16: note = Description of sequence: VH CDR3
AKDRWGYSSGWHNFDF

SEQ ID NO: 6 - protein, 12 aa, Homo sapiens
  REGION 1..12: note = Description of sequence: VL CDR1
QSVLRSGNNKND

SEQ ID NO: 7 - skipped

SEQ ID NO: 8 - protein, 9 aa, Homo sapiens
  REGION 1..9: note = Description of sequence: VL CHR3
QQSSSEPIT

SEQ ID NO: 9 - protein, 123 aa, Homo sapiens
  REGION 1..123: note = Description of sequence: Full VH
EVQLLESGGGLVQPGGSLRLSCAASGFTFSDYYMSWVRQAPGKGLEWVSGIYGGGGSTRYADSVKGRFTISRDNSKNTLYLQMNSLRAEDTAVYYCAKDRWGYSSGWHNFDFWGQGTLVTVSS

SEQ ID NO: 10 - protein, 369 aa, Homo sapiens
  REGION 1..369: note = Description of sequence: DNA for Full VH
GAAGTGCAGCTGCTGGAATCTGGCGGAGGACTGGTTCAACCTGGCGGCTCTCTGAGACTGTCTTGTGCCGCCAGCGGCTTCACCTTCAGCGACTACTACATGAGCTGGGTCCGACAGGCCCCTGGCAAAGGACTTGAATGGGTGTCCGGCATCTATGGCGGCGGAGGCTCTACAAGATACGCCGACTCTGTGAAGGGCAGATTCACCATCAGCCGGGACAACAGCAAGAACACCCTGTACCTGCAGATGAACAGCCTGAGAGCCGAGGACACCGCCGTGTACTACTGCGCCAAAGACAGATGGGGCTACAGCAGCGGCTGGCACAACTTCGATTTTTGGGGCCAGGGCACCCTGGTCACCGTTTCTTCT

SEQ ID NO: 11 - protein, 9 aa, Homo sapiens
  REGION 1..9: note = Description of sequence: VL CDR3
QQSYNEPIT

SEQ ID NO: 12 - protein, 113 aa, Homo sapiens
  REGION 1..113: note = Description of sequence: Full VL
DIVMTQSPDSLAVSLGERATINCKSSQSVLRSYNNKNDLAWYQQKPGQPPKLLIYWASTRESGVPDRFSGSGSGTDFTLTISSLQAEDVAVYYCQQSYNEPITFGQGTKVEIK

SEQ ID NO: 13 - protein, 12 aa, Homo sapiens
  REGION 1..12: note = Description of sequence: VL CDR1
QSVLYSYNNKND

SEQ ID NO: 14 - protein, 9 aa, Homo sapiens
  REGION 1..9: note = Description of sequence: VL CDR3
QQSYEEPIT

SEQ ID NO: 15 - protein, 113 aa, Homo sapiens
  REGION 1..113: note = Description of sequence: Full VL
DIVMTQSPDSLAVSLGERATINCKSSQSVLYSYNNKNDLAWYQQKPGQPPKLLIYWASTRESGVPDRFSGSGSGTDFTLTISSLQAEDVAVYYCQQSYEEPITFGQGTKVEIK

SEQ ID NO: 16 - protein, 9 aa, Homo sapiens
  REGION 1..9: note = Description of sequence: VL CDR3
QQSYYSPIT

SEQ ID NO: 17 - protein, 113 aa, Homo sapiens
  REGION 1..113: note = Description of sequence: Full VL
DIVMTQSPDSLAVSLGERATINCKSSQSVLRSYNNKNDLAWYQQKPGQPPKLLIYWASTRESGVPDRFSGSGSGTDFTLTISSLQAEDVAVYYCQQSYYSPITFGQGTKVEIK

SEQ ID NO: 18 - protein, 12 aa, Homo sapiens
  REGION 1..12: note = Description of sequence: VL CDR1
QSVLRSGNNKNR

SEQ ID NO: 19 - protein, 9 aa, Homo sapiens
  REGION 1..9: note = Description of sequence: VL CDR3
QQYTRRPIT

SEQ ID NO: 20 - protein, 113 aa, Homo sapiens
  REGION 1..113: note = Description of sequence: Full VL
DIVMTQSPDSLAVSLGERATINCKSSQSVLRSGNNKNRLAWYQQKPGQPPKLLIYWASTRESGVPDRFSGSGSGTDFTLTISSLQAEDVAVYYCQQYTRRPITFGQGTKVEIK

SEQ ID NO: 21 - protein, 9 aa, Homo sapiens
  REGION 1..9: note = Description of sequence: VL CDR3
QQSYSEPLT

SEQ ID NO: 22 - protein, 113 aa, Homo sapiens
  REGION 1..113: note = Description of sequence: Full VL
DIVMTQSPDSLAVSLGERATINCKSSQSVLRSGNNKNDLAWYQQKPGQPPKLLIYWASTRESGVPDRFSGSGSGTDFTLTISSLQAEDVAVYYCQQSYSEPLTFGQGTKVEIK

SEQ ID NO: 23 - protein, 9 aa, Homo sapiens
  REGION 1..9: note = Description of sequence: VL CDR3
QQSYNAPLT

SEQ ID NO: 24 - protein, 113 aa, Homo sapiens
  REGION 1..113: note = Description of sequence: Full VL
DIVMTQSPDSLAVSLGERATINCKSSQSVLRSYNNKNDLAWYQQKPGQPPKLLIYWASTRESGVPDRFSGSGSGTDFTLTISSLQAEDVAVYYCQQSYNAPLTFGQGTKVEIK

SEQ ID NO: 25 - protein, 9 aa, Homo sapiens
  REGION 1..9: note = Description of sequence: VL CDR3
QQSYNSPIT

SEQ ID NO: 26 - protein, 113 aa, Homo sapiens
  REGION 1..113: note = Description of sequence: Full VL
DIVMTQSPDSLAVSLGERATINCKSSQSVLRSYNNKNDLAWYQQKPGQPPKLLIYWASTRESGVPDRFSGSGSGTDFTLTISSLQAEDVAVYYCQQSYNSPITFGQGTKVEIK

SEQ ID NO: 27 - protein, 9 aa, Homo sapiens
  REGION 1..9: note = Description of sequence: VL CDR3
QQSYSSPIT

SEQ ID NO: 28 - protein, 113 aa, Homo sapiens
  REGION 1..113: note = Description of sequence: Full VL
DIVMTQSPDSLAVSLGERATINCKSSQSVLRSYNNKNDLAWYQQKPGQPPKLLIYWASTRESGVPDRFSGSGSGTDFTLTISSLQAEDVAVYYCQQSYSSPITFGQGTKVEIK

SEQ ID NO: 29 - protein, 8 aa, Homo sapiens
  REGION 1..8: note = Description of sequence: VH CDR2
ISSSGSTI

SEQ ID NO: 30 - protein, 12 aa, Homo sapiens
  REGION 1..12: note = Description of sequence: VH CDR3
ARGPLTADAFDI

SEQ ID NO: 31 - protein, 7 aa, Homo sapiens
  REGION 1..7: note = Description of sequence: VL CDR1
SNIGSNT

SEQ ID NO: 32 - skipped

SEQ ID NO: 33 - protein, 11 aa, Homo sapiens
  REGION 1..11: note = Description of sequence: VL CDR3
ATWDDTRNGPE

SEQ ID NO: 34 - protein, 119 aa, Homo sapiens
  REGION 1..119: note = Description of sequence: Full VH
EVQLVQSGGGLVKPGGSLRLSCAASGFTFSDYYMSWIRQAPGKGLEWVSYISSSGSTIYYADSVKGRFTISRDNAKNSLYLQMNSLRAEDTAVYYCARGPLTADAFDIWGQGTMVTVSS

SEQ ID NO: 35 - protein, 110 aa, Homo sapiens
  REGION 1..110: note = Description of sequence: Full VL
QSVLTQPPSASGTPGQRVTISCSGSSSNIGSNTVNWYQQLPGTAPKLLIYNNNQRPSGVPDRFSGSKSGTSAALAISGLQSEDEADYHCATWDDTRNGPEFGGGTKLTVL

SEQ ID NO: 36 - protein, 8 aa, Homo sapiens
  REGION 1..8: note = Description of sequence: VH CDR1
GFTFSSTA

SEQ ID NO: 37 - protein, 8 aa, Homo sapiens
  REGION 1..8: note = Description of sequence: VH CDR2
ISGSGSYT

SEQ ID NO: 38 - protein, 18 aa, Homo sapiens
  REGION 1..18: note = Description of sequence: VH CDR3
ARHHPDDYGDYVRDAFDI

SEQ ID NO: 39 - protein, 7 aa, Homo sapiens
  REGION 1..7: note = Description of sequence: VL CDR1
QSVSHSY

SEQ ID NO: 40 - skipped

SEQ ID NO: 41 - protein, 9 aa, Homo sapiens
  REGION 1..9: note = Description of sequence: VL CDR3
QQYEEYPLT

SEQ ID NO: 42 - protein, 125 aa, Homo sapiens
  REGION 1..125: note = Description of sequence: Full VH
EVQLLESGGGLVQPGGSLRLSCAASGFTFSSTAMSWVRQAPGKGLEWVSEISGSGSYTSYADSVKGRFTISRDNSKNTLYLQMNSLRAEDTAVYYCARHHPDDYGDYVRDAFDIWGQGTMVTVSS

SEQ ID NO: 43 - protein, 108 aa, Homo sapiens
  REGION 1..108: note = Description of sequence: Full VL
EIVLTQSPGTLSLSPGERATLSCRASQSVSHSYLAWYQQKPGQAPRLLIYGASSRATGIPDRFSGSGSGTDFTLTISRLEPEDFAVYYCQQYEEYPLTFGQGTKVEIK

SEQ ID NO: 44 - protein, 19 aa, Homo sapiens
  REGION 1..19: note = Description of sequence: G4S Linker
GGGGSGGGGSGGGSGGGGS

SEQ ID NO: 45 - protein, 15 aa, Homo sapiens
  REGION 1..15: note = Description of sequence: G4S Linker
GGGGSGGGGSGGGGS

SEQ ID NO: 46 - protein, 24 aa, Homo sapiens
  REGION 1..24: note = Description of sequence: G4S Linker
GGGGSGGGGSGGGGSGGGSGGGGS

SEQ ID NO: 47 - protein, 29 aa, Homo sapiens
  REGION 1..29: note = Description of sequence: G4S Linker
GGGGSGGGGSGGGGSGGGGSGGGSGGGGS

SEQ ID NO: 48 - protein, 5 aa, Homo sapiens
  REGION 1..5: note = Description of sequence: G4S Linker
GGGGS

SEQ ID NO: 49 - protein, 10 aa, Homo sapiens
  REGION 1..10: note = Description of sequence: G4S Linker
GGGGSGGGGS

SEQ ID NO: 50 - protein, 330 aa, Homo sapiens [sequence omitted]

SEQ ID NO: 51 - protein, 107 aa, Homo sapiens [sequence omitted]

SEQ ID NO: 52 - DNA, 369 bp, Homo sapiens
  misc_feature 1..369: note = Description of sequence: DNA for Full VH
  source 1..369: mol_type = genomic DNA
gaagtgcagctgctggaatctggcggaggactggttcaacctggcggctctctgagactgtcttgtgccgccagcggcttcaccttcagcgactactacatgagctgggtccgacaggcccctggcaaaggacttgaatgggtgtccggcatctatggcggcggaggctctacaagatacgccgactctgtgaagggcagattcaccatcagccgggacaacagcaagaacaccctgtacctgcagatgaacagcctgagagccgaggacaccgccgtgtactactgcgccaaagacagatggggctacagcagcggctggcacaacttcgatttttggggccagggcaccctggtcaccgtttcttct

SEQ ID NO: 53 - DNA, 339 bp, Homo sapiens
  misc_feature 1..339: note = Description of sequence: DNA for Full VL
  source 1..339: mol_type = genomic DNA
gacatcgtgatgacacagagccctgatagcctggccgtgtctctgggagagagagccaccatcaactgcaagagcagccagagcgtgctgcggagcggcaacaacaagaatgacctggcctggtatcagcagaagcccggccaacctcctaagctgctgatctactgggccagcaccagagaaagcggcgtgcccgatagattttctggcagcggctctggcaccgacttcaccctgacaattagctccctgcaggccgaggatgtggccgtgtactactgtcagcagagcagcagcgagcccatcacatttggccagggcaccaaggtggaaatcaag

SEQ ID NO: 54 - DNA, 357 bp, Homo sapiens
  misc_feature 1..357: note = Description of sequence: DNA for Full VH
  source 1..357: mol_type = genomic DNA
gaagtgcagctggttcagtctggcggcggacttgtgaaacctggcggaagcctgagactgagctgtgccgcttccggcttcaccttcagcgactactacatgagctggatcagacaggcccctggcaaaggcctggaatgggtgtcctacatcagcagcagcggcagcaccatctactacgccgatagcgtgaagggcagattcaccatcagccgggacaacgccaagaacagcctgtacctgcagatgaactccctgagagccgaggacaccgccgtgtactattgtgccagaggacctctgaccgccgacgcctttgatatctggggccagggcacaatggtcaccgtgtcatct

SEQ ID NO: 55 - DNA, 330 bp, Homo sapiens
  misc_feature 1..330: note = Description of sequence: DNA for Full VL
  source 1..330: mol_type = genomic DNA
cagtctgtgctgacacagcctccatctgcctctggcacacctggccagagagtgaccatcagctgtagcggcagcagcagcaacatcggcagcaacaccgtgaactggtatcagcagctgcctggcacagcccctaaactgctgatctacaacaacaaccagcggcctagcggcgtgcccgatagattttctggcagcaagagcggcacaagcgccgctctggctatttctggactgcagagcgaggacgaggccgattaccattgtgccacctgggacgacaccagaaacggccctgaatttggcggaggcaccaagctgacagtgctg

SEQ ID NO: 56 - DNA, 375 bp, Homo sapiens
  misc_feature 1..375: note = Description of sequence: DNA for Full VH
  source 1..375: mol_type = genomic DNA
gaagtgcagctgctggaatctggcggaggactggttcaacctggcggctctctgagactgtcttgtgccgccagcggcttcacctttagcagcacagccatgagctgggtccgacaggctcctggcaaaggccttgaatgggtgtccgagatcagcggcagcggctcttacacaagctacgccgatagcgtgaagggcagattcaccatcagccgggacaacagcaagaacaccctgtacctgcagatgaacagcctgagagccgaggacaccgccgtgtactactgtgccagacatcaccccgacgactacggcgattatgtgcgggacgccttcgatatctggggccagggaacaatggtcaccgtgtctagt

SEQ ID NO: 57 - DNA, 324 bp, Homo sapiens
  misc_feature 1..324: note = Description of sequence: DNA for Full VL
  source 1..324: mol_type = genomic DNA
gagatcgtgctgacacagagccctggcacactgtcactgtctccaggcgaaagagccacactgagctgtagagccagccagagcgtgtcccactcttacctggcttggtatcagcagaagcccggacaggctcccagactgctgatctatggcgcctcttctagagccacaggcatccccgatagattcagcggctctggcagcggcaccgatttcaccctgacaatcagcagactggaacccgaggacttcgccgtgtactactgccagcagtacgaggaataccctctgacctttggccagggcaccaaggtggaaatcaag

(19) United States
(12) Patent Application Publication
(10) Pub. No.: US 2024/0360202 A1
(43) Pub. Date: Oct. 31, 2024

(54) ANTI-HPV ANTIBODIES AND USES THEREOF

(71) Applicants: Memorial Sloan-Kettering Cancer Center, New York, NY (US); Sloan-Kettering Institute for Cancer Research, New York, NY (US); Memorial Hospital for Cancer and Allied Diseases, New York, NY (US); Tri-Institutional Therapeutics Discovery Institute, Inc., New York, NY (US)

(72) Inventors: David A. Scheinberg, New York, NY (US); Tao Dao, New York, NY (US); Mary Ann Pohl, Astoria, NY (US); David Andrew, Sandy Hook, CT (US); David Andrew, Sandy Hook, CT (US)

(73) Assignees: Memorial Sloan-Kettering Cancer Center, New York, NY (US); Sloan-Kettering Institute for Cancer Research, New York, NY (US); Memorial Hospital for Cancer and Allied Diseases, New York, NY (US); Tri-Institutional Therapeutics Discovery Institute, Inc., New York, NY (US)

(21) Appl. No.: 18/760,674

(22) Filed: Jul. 1, 2024

Related U.S. Application Data
(63) Continuation of application No. PCT/US2023/010189, filed on Jan. 5, 2023.
(60) Provisional application No. 63/305,649, filed on Feb. 1, 2022.
(60) Provisional application No. 63/296,657, filed on Jan. 5, 2022.

(51) Int. Cl. C07K 16/08; A61K 47/68

(52) U.S. Cl. CPC C07K 16/084; A61K 47/6839

(57) ABSTRACT

The presently disclosed subject matter provides antibodies that mimic TCR recognition of HPV-derived epitopes presented by HLA class I molecules, antigen-recognizing receptors that target HPV-derived epitopes presented by HLA class I molecules, and methods of using such antibodies.

### CROSS-REFERENCE TO RELATED APPLICATIONS

This application is a Continuation application of International Patent Application No. PCT/US23/10189, filed Jan. 5, 2023, which claims priority to U.S. Provisional Patent Application No. 63/296,657, filed on Jan. 5, 2022, and to U.S. Provisional Patent Application No. 63/305,649, filed on Feb. 1, 2022, the content of each of which is incorporated by reference in its entirety, and to each of which priority is claimed.

### STATEMENT REGARDING FEDERALLY-SPONSORED RESEARCH

This invention was made with government support under CA055349, CA023766, CA241894 and CA008748 awarded by the National Institutes of Health. The government has certain rights in the invention.

### SEQUENCE LISTING

A Sequence Listing conforming to the rules of WIPO Standard ST.26 is hereby incorporated by reference. Said Sequence Listing has been filed as an electronic document via EFS-Web in ASCII format encoded as XML. The electronic document, created on Jul. 1, 2024, is entitled “0727341594_ST26.xml”, and is 73,657 bytes in size.

### 1. FIELD OF THE INVENTION

The presently disclosed subject matter relates to antibodies that mimic TCR recognition of HPV-derived epitopes presented by HLA class I molecules, antigen-recognizing receptors that target HPV-derived epitopes presented by HLA class I molecules, and methods of using such antibodies.

### 2. BACKGROUND OF THE INVENTION

High risk HPV causes malignant transformation following persistent infection and is the cause of cervical cancer and other infection-related cancers. Prophylactic vaccination-induced antibodies only neutralize virus particles before infections but have no therapeutic efficacy for the cryptic oncogenic proteins remaining or the later induced cancers. HPV E6 and E7 viral oncogenic proteins are not currently druggable by small molecules and are intracellular and hence, inaccessible to therapeutic antibodies. T cells have been attributed to the natural clearance of most infected cells because T cells recognize and destroy the infected cells that present viral protein-derived peptide fragments complexed with HLA class I and class II molecules on the cancer cell surface. Given the significant role of HPV in diseases, novel immunotherapies targeting HPV-derived proteins are desired.

### 3. SUMMARY OF THE INVENTION

The presently disclosed subject matter provides antibodies that bind to Human papillomavirus (HPV) epitopes and methods of using such antibodies. The presently disclosed subject matter provides antibodies that mimic TCR recognition of HPV-derived epitopes presented by HLA class I molecules. These novel antibodies provide an effective immunotherapeutic approach targeting HPV-induced malignancies.

In certain embodiments, the presently disclosed antibodies or antigen-binding fragments thereof that bind to an HPV epitope. In certain embodiments, the antibodies antigen-binding fragments thereof bind to an HPV-E7 epitope. In certain embodiments, the antibodies antigen-binding fragments thereof bind to an HPV-E7-derived CD8 T cell epitope. In certain embodiments, the epitope comprises or consists of the amino acid sequence set forth in SEQ ID NO: 1. In certain embodiments, the epitope is expressed in the context of a human leukocyte antigen (HLA). In certain embodiments, the HLA is an HLA-A. In certain embodiments, the HLA is an HLA-A*02:01. In certain embodiments, the epitope induces a CD8 T cell response in the context of HLA-A*02:01 molecule.

In certain embodiments, the presently disclosed subject matter provides antibodies or antigen-binding fragments thereof that binds to an HPV epitope bound to a human major histocompatibility complex (MHC) molecule. In certain embodiments, the MHC molecule is a human leukocyte antigen (HLA) molecule. In certain embodiments, the HLA molecule is an HLA class I molecule. In certain embodiments, the HLA class I molecule is HLA-A. In certain embodiments, the HLA-A is HLA-A2. In certain embodiments, the HLA-A2 is HLA-A*02:01. In certain embodiments, the HPV epitope is an E7 epitope. In certain embodiments, the E7 epitope comprises or consists of the amino acid sequence set forth in SEQ ID NO: 1.

In certain embodiments, the antibody or antigen-binding fragment thereof comprises a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 9, SEQ ID NO: 34, or SEQ ID NO: 42. In certain embodiments, the antibody or antigen-binding fragment thereof comprises a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 10, SEQ ID NO: 12, SEQ ID NO: 15, SEQ ID NO: 17, SEQ ID NO: 20, SEQ ID NO: 22, SEQ ID NO: 24, SEQ ID NO: 26, SEQ ID NO: 28, SEQ ID NO: 35, or SEQ ID NO: 43.

In certain embodiments, the antibody or antigen-binding fragment thereof comprises:
(a) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 9, SEQ ID NO: 34, or SEQ ID NO: 41; and
(b) a light chain variable region comprising an amino acid sequence that is at least about 80%, at least about 85%, at least about 90%, at least about 95%, at least about 96%, at least about 97%, at least about 98%, at least about 99% at least about 100% homologous or identical to the amino acid sequence set forth in SEQ ID NO: 10, SEQ ID NO: 12, SEQ ID NO: 15, SEQ ID NO: 17, SEQ ID NO: 20, SEQ ID NO: 22, SEQ ID NO: 24, SEQ ID NO: 26, SEQ ID NO: 28, SEQ ID NO: 35, or SEQ ID NO: 43.

In certain embodiments, the heavy chain variable region and the light chain variable region are selected from the group consisting of:
(a) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 9, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 10;
(b) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 9, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 12;
(c) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 9, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 15;
(d) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 9, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 17;
(e) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 9, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 20;
(f) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 9, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 22;
(g) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 9, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 24;
(h) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 9, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 26;
(i) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 9, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 28;
(j) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 34, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 35; and
(k) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 42, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 43.

In certain embodiments, the heavy chain variable region comprises the amino acid sequence set forth in SEQ ID NO: 9, SEQ ID NO: 34, or SEQ ID NO: 42. In certain embodiments, light chain variable region comprises the amino acid sequence set forth in SEQ ID NO: 10, SEQ ID NO: 12, SEQ ID NO: 15, SEQ ID NO: 17, SEQ ID NO: 20, SEQ ID NO: 22, SEQ ID NO: 24, SEQ ID NO: 26, SEQ ID NO: 28, SEQ ID NO: 35, or SEQ ID NO: 43.

In certain embodiments, the antibody or antigen-binding fragment thereof comprises:
(a) a heavy chain variable region comprising the amino acid sequence set forth in SEQ ID NO: 9, SEQ ID NO: 34, or SEQ ID NO: 42; and
(b) a light chain variable region comprising the amino acid sequence set forth in SEQ ID NO: 10, SEQ ID NO: 12, SEQ ID NO: 15, SEQ ID NO: 17, SEQ ID NO: 20, SEQ ID NO: 22, SEQ ID NO: 24, SEQ ID NO: 26, SEQ ID NO: 28, SEQ ID NO: 35, or SEQ ID NO: 43.

In certain embodiments,
(a) the heavy chain variable region comprises the amino acid sequence set forth in SEQ ID NO: 9, and the light chain variable region comprises the amino acid sequence set forth in SEQ ID NO: 10;
(b) the heavy chain variable region comprises the amino acid sequence set forth in SEQ ID NO: 9, and the light chain variable region comprises the amino acid sequence set forth in SEQ ID NO: 12;
(c) the heavy chain variable region comprises the amino acid sequence set forth in SEQ ID NO: 9, and the light chain variable region comprises the amino acid sequence set forth in SEQ ID NO: 15;
(d) the heavy chain variable region comprises the amino acid sequence set forth in SEQ ID NO: 9, and the light chain variable region comprises the amino acid sequence set forth in SEQ ID NO: 17;
(e) the heavy chain variable region comprises the amino acid sequence set forth in SEQ ID NO: 9, and the light chain variable region comprises the amino acid sequence set forth in SEQ ID NO: 20;
(f) the heavy chain variable region comprises the amino acid sequence set forth in SEQ ID NO: 9, and the light chain variable region comprises the amino acid sequence set forth in SEQ ID NO: 22;
(g) the heavy chain variable region comprises the amino acid sequence set forth in SEQ ID NO: 9, and the light chain variable region comprises the amino acid sequence set forth in SEQ ID NO: 24;
(h) the heavy chain variable region comprises the amino acid sequence set forth in SEQ ID NO: 9, and the light chain variable region comprises the amino acid sequence set forth in SEQ ID NO: 26;
(i) the heavy chain variable region comprises the amino acid sequence set forth in SEQ ID NO: 9, and the light chain variable region comprises the amino acid sequence set forth in SEQ ID NO: 28;
(j) the heavy chain variable region comprises the amino acid sequence set forth in SEQ ID NO: 34, and the light chain variable region comprises the amino acid sequence set forth in SEQ ID NO: 35; or
(k) the heavy chain variable region comprises the amino acid sequence set forth in SEQ ID NO: 42, and the light chain variable region comprises the amino acid sequence set forth in SEQ ID NO: 43.

In certain embodiments, the heavy chain variable region comprises the amino acid sequence set forth in SEQ ID NO: 9, and the light chain variable region comprises the amino acid sequence set forth in SEQ ID NO: 10.

In certain embodiments, the antibody or antigen-binding fragment thereof comprises a heavy chain variable region that comprises CDR1, CDR2, and CDR3 domains; and a light chain variable region that comprises CDR1, CDR2, and CDR3 domains. In certain embodiments, the heavy chain variable region and light chain variable region CDR3 domains are selected from the group consisting of:
(a) a heavy chain variable region CDR3 comprising the amino acid sequence set forth in SEQ ID NO: 5 or a conservative modification thereof; and a light chain variable region CDR3 comprising the amino acid sequence set forth in SEQ ID NO: 8 or a conservative modification thereof;
(b) a heavy chain variable region CDR3 comprising the amino acid sequence set forth in SEQ ID NO: 5 or a conservative modification thereof; and a light chain variable region CDR3 comprising the amino acid sequence set forth in SEQ ID NO: 11 or a conservative modification thereof;
(c) a heavy chain variable region CDR3 comprising the amino acid sequence set forth in SEQ ID NO: 5 or a conservative modification thereof; and a light chain variable region CDR3 comprising the amino acid sequence set forth in SEQ ID NO: 14 or a conservative modification thereof;
(d) a heavy chain variable region CDR3 comprising the amino acid sequence set forth in SEQ ID NO: 5 or a conservative modification thereof; and a light chain variable region CDR3 comprising the amino acid sequence set forth in SEQ ID NO: 16 or a conservative modification thereof;
(e) a heavy chain variable region CDR3 comprising the amino acid sequence set forth in SEQ ID NO: 5 or a conservative modification thereof; and a light chain variable region CDR3 comprising the amino acid sequence set forth in SEQ ID NO: 19 or a conservative modification thereof;
(f) a heavy chain variable region CDR3 comprising the amino acid sequence set forth in SEQ ID NO: 5 or a conservative modification thereof; and a light chain variable region CDR3 comprising the amino acid sequence set forth in SEQ ID NO: 21 or a conservative modification thereof;
(g) a heavy chain variable region CDR3 comprising the amino acid sequence set forth in SEQ ID NO: 5 or a conservative modification thereof; and a light chain variable region CDR3 comprising the amino acid sequence set forth in SEQ ID NO: 23 or a conservative modification thereof;
(h) a heavy chain variable region CDR3 comprising the amino acid sequence set forth in SEQ ID NO: 5 or a conservative modification thereof; and a light chain variable region CDR3 comprising the amino acid sequence set forth in SEQ ID NO: 25 or a conservative modification thereof;
(i) a heavy chain variable region CDR3 comprising the amino acid sequence set forth in SEQ ID NO: 5 or a conservative modification thereof; and a light chain variable region CDR3 comprising the amino acid sequence set forth in SEQ ID NO: 27 or a conservative modification thereof;
(j) a heavy chain variable region CDR3 comprising the amino acid sequence set forth in SEQ ID NO: 30 or a conservative modification thereof; and a light chain variable region CDR3 comprising the amino acid sequence set forth in SEQ ID NO: 33 or a conservative modification thereof; or
(k) a heavy chain variable region CDR3 comprising the amino acid sequence set forth in SEQ ID NO: 38 or a conservative modification thereof; and a light chain variable region CDR3 comprising the amino acid sequence set forth in SEQ ID NO: 41 or a conservative modification thereof.

In certain embodiments, the heavy chain variable region and light chain variable region CDR2 domains are selected from:
(a) a heavy chain variable region CDR2 comprising the amino acid sequence set forth in SEQ ID NO: 4 or a conservative modification thereof; and a light chain variable region CDR2 comprising the amino acid sequence set forth in SEQ ID NO: 7 or a conservative modification thereof;
(b) a heavy chain variable region CDR2 comprising the amino acid sequence set forth in SEQ ID NO: 29 or a conservative modification thereof; and a light chain variable region CDR2 comprising the amino acid sequence set forth in SEQ ID NO: 32 or a conservative modification thereof; or
(c) a heavy chain variable region CDR2 comprising the amino acid sequence set forth in SEQ ID NO: 37 or a conservative modification thereof; and a light chain variable region CDR2 comprising the amino acid sequence set forth in SEQ ID NO: 40 or a conservative modification thereof.

In certain embodiments, the heavy chain variable region and light chain variable region CDR1 domains are selected from the group consisting of:
(a) a heavy chain variable region CDR1 comprising the amino acid sequence set forth in SEQ ID NO: 3 or a conservative modification thereof; and a light chain variable region CDR1 comprising the amino acid sequence set forth in SEQ ID NO: 6 or a conservative modification thereof;
(b) a heavy chain variable region CDR1 comprising the amino acid sequence set forth in SEQ ID NO: 3 or a conservative modification thereof; and a light chain variable region CDR1 comprising the amino acid sequence set forth in SEQ ID NO: 13 or a conservative modification thereof;
(c) a heavy chain variable region CDR1 comprising the amino acid sequence set forth in SEQ ID NO: 3 or a conservative modification thereof; and a light chain variable region CDR1 comprising the amino acid sequence set forth in SEQ ID NO: 18 or a conservative modification thereof;
(d) a heavy chain variable region CDR1 comprising the amino acid sequence set forth in SEQ ID NO: 3 or a conservative modification thereof; and a light chain variable region CDR1 comprising the amino acid sequence set forth in SEQ ID NO: 31 or a conservative modification thereof; or
(e) a heavy chain variable region CDR1 comprising the amino acid sequence set forth in SEQ ID NO: 36 or a conservative modification thereof; and a light chain variable region CDR1 comprising the amino acid sequence set forth in SEQ ID NO: 39 or a conservative modification thereof.

In certain embodiments, one or more of the CDR sequences have up to about 5 amino acid substitutions. In certain embodiments, one or more of the CDR sequences have up to about 3 amino acid substitutions.

In certain embodiments,
(a) the heavy chain variable region comprises a CDR1 comprising the amino acid sequence set forth in SEQ ID NO: 3, a CDR2 comprising the amino acid sequence set forth in SEQ ID NO: 4, and a CDR3 comprising the amino acid sequence set forth in SEQ ID NO: 5;
(b) the heavy chain variable region comprises a CDR1 comprising the amino acid sequence set forth in SEQ ID NO: 3, a CDR2 comprising the amino acid sequence set forth in SEQ ID NO: 29, and a CDR3 comprising the amino acid sequence set forth in SEQ ID NO: 30; or
(c) the heavy chain variable region comprises a CDR1 comprising the amino acid sequence set forth in SEQ ID NO: 36, a CDR2 comprising the amino acid sequence set forth in SEQ ID NO: 37, and a CDR3 comprising the amino acid sequence set forth in SEQ ID NO: 38.

In certain embodiments,
(a) the light chain variable region comprises a CDR1 comprising the amino acid sequence set forth in SEQ ID NO: 6, a CDR2 comprising the amino acid sequence set forth in SEQ ID NO: 7, and a CDR3 comprising the amino acid sequence set forth in SEQ ID NO: 8;
(b) the light chain variable region comprises a CDR1 comprising the amino acid sequence set forth in SEQ ID NO: 6, a CDR2 comprising the amino acid sequence set forth in SEQ ID NO: 7, and a CDR3 comprising the amino acid sequence set forth in SEQ ID NO: 11;
(c) the light chain variable region comprises a CDR1 comprising the amino acid sequence set forth in SEQ ID NO: 13, a CDR2 comprising the amino acid sequence set forth in SEQ ID NO: 7, and a CDR3 comprising the amino acid sequence set forth in SEQ ID NO: 14;
(d) the light chain variable region comprises a CDR1 comprising the amino acid sequence set forth in SEQ ID NO: 6; a CDR2 comprising the amino acid sequence set forth in SEQ ID NO: 7; and a CDR3 comprising the amino acid sequence set forth in SEQ ID NO: 16;
(e) the light chain variable region comprises a CDR1 comprising the amino acid sequence set forth in SEQ ID NO: 18, a CDR2 comprising the amino acid sequence set forth in SEQ ID NO: 7, and a CDR3 comprising the amino acid sequence set forth in SEQ ID NO: 19;
(f) the light chain variable region comprises a CDR1 comprising the amino acid sequence set forth in SEQ ID NO: 6, a CDR2 comprising the amino acid sequence set forth in SEQ ID NO: 7, and a CDR3 comprising the amino acid sequence set forth in SEQ ID NO: 21;
(g) the light chain variable region comprises a CDR1 comprising the amino acid sequence set forth in SEQ ID NO: 6, a CDR2 comprising the amino acid sequence set forth in SEQ ID NO: 7, and a CDR3 comprising the amino acid sequence set forth in SEQ ID NO: 23;
(h) the light chain variable region comprises a CDR1 comprising the amino acid sequence set forth in SEQ ID NO: 6, a CDR2 comprising the amino acid sequence set forth in SEQ ID NO: 7, and a CDR3 comprising the amino acid sequence set forth in SEQ ID NO: 25;
(i) the light chain variable region comprises a CDR1 comprising the amino acid sequence set forth in SEQ ID NO: 6, a CDR2 comprising the amino acid sequence set forth in SEQ ID NO: 7, and a CDR3 comprising the amino acid sequence set forth in SEQ ID NO: 27;
(j) the light chain variable region comprises a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33; or
(k) the light chain variable region comprises a CDR1 comprising the amino acid sequence set forth in SEQ ID NO: 39, a CDR2 comprising the amino acid sequence set forth in SEQ ID NO: 40, and a CDR3 comprising the amino acid sequence set forth in SEQ ID NO: 41.

In certain embodiments,
(a) the heavy chain variable region comprises a CDR1 comprising the amino acid sequence set forth in SEQ ID NO: 3, a CDR2 comprising the amino acid sequence set forth in SEQ ID NO: 4, and a CDR3 comprising the amino acid sequence set forth in SEQ ID NO: 5; the light chain variable region comprises a CDR1 comprising the amino acid sequence set forth in SEQ ID NO: 6, a CDR2 comprising the amino acid sequence set forth in SEQ ID NO: 7, and a CDR3 comprising the amino acid sequence set forth in SEQ ID NO: 8;
(b) the heavy chain variable region comprises a CDR1 comprising the amino acid sequence set forth in SEQ ID NO: 3, a CDR2 comprising the amino acid sequence set forth in SEQ ID NO: 4, and a CDR3 comprising the amino acid sequence set forth in SEQ ID NO: 5; the light chain variable region comprises a CDR1 comprising the amino acid sequence set forth in SEQ ID NO: 6, a CDR2 comprising the amino acid sequence set forth in SEQ ID NO: 7, and a CDR3 comprising the amino acid sequence set forth in SEQ ID NO: 11;
(c) the heavy chain variable region comprises a CDR1 comprising the amino acid sequence set forth in SEQ ID NO: 3, a CDR2 comprising the amino acid sequence set forth in SEQ ID NO: 4, and a CDR3 comprising the amino acid sequence set forth in SEQ ID NO: 5; the light chain variable region comprises a CDR1 comprising the amino acid sequence set forth in SEQ ID NO: 13, a CDR2 comprising the amino acid sequence set forth in SEQ ID NO: 7, and a CDR3 comprising the amino acid sequence set forth in SEQ ID NO: 14;
(d) the heavy chain variable region comprises a CDR1 comprising the amino acid sequence set forth in SEQ ID NO: 3, a CDR2 comprising the amino acid sequence set forth in SEQ ID NO: 4, and a CDR3 comprising the amino acid sequence set forth in SEQ ID NO: 5; the light chain variable region comprises a CDR1 comprising the amino acid sequence set forth in SEQ ID NO: 6, a CDR2 comprising the amino acid sequence set forth in SEQ ID NO: 7, and a CDR3 comprising the amino acid sequence set forth in SEQ ID NO: 16;
(e) the heavy chain variable region comprises a CDR1 comprising the amino acid sequence set forth in SEQ ID NO: 3, a CDR2 comprising the amino acid sequence set forth in SEQ ID NO: 4, and a CDR3 comprising the amino acid sequence set forth in SEQ ID NO: 5; the light chain variable region comprises a CDR1 comprising the amino acid sequence set forth in SEQ ID NO: 18, a CDR2 comprising the amino acid sequence set forth in SEQ ID NO: 7, and a CDR3 comprising the amino acid sequence set forth in SEQ ID NO: 19;
(f) the heavy chain variable region comprises a CDR1 comprising the amino acid sequence set forth in SEQ ID NO: 3, a CDR2 comprising the amino acid sequence set forth in SEQ ID NO: 4, and a CDR3 comprising the amino acid sequence set forth in SEQ ID NO: 5; the light chain variable region comprises a CDR1 comprising the amino acid sequence set forth in SEQ ID NO: 6, a CDR2 comprising the amino acid sequence set forth in SEQ ID NO: 7, and a CDR3 comprising the amino acid sequence set forth in SEQ ID NO: 21;
(g) the heavy chain variable region comprises a CDR1 comprising the amino acid sequence set forth in SEQ ID NO: 3, a CDR2 comprising the amino acid sequence set forth in SEQ ID NO: 4, and a CDR3 comprising the amino acid sequence set forth in SEQ ID NO: 5; the light chain variable region comprises a CDR1 comprising the amino acid sequence set forth in SEQ ID NO: 6, a CDR2 comprising the amino acid sequence set forth in SEQ ID NO: 7, and a CDR3 comprising the amino acid sequence set forth in SEQ ID NO: 23;
(h) the heavy chain variable region comprises a CDR1 comprising the amino acid sequence set forth in SEQ ID NO: 3, a CDR2 comprising the amino acid sequence set forth in SEQ ID NO: 4, and a CDR3 comprising the amino acid sequence set forth in SEQ ID NO: 5; the light chain variable region comprises a CDR1 comprising the amino acid sequence set forth in SEQ ID NO: 6, a CDR2 comprising the amino acid sequence set forth in SEQ ID NO: 7, and a CDR3 comprising the amino acid sequence set forth in SEQ ID NO: 25;
(i) the heavy chain variable region comprises a CDR1 comprising the amino acid sequence set forth in SEQ ID NO: 3, a CDR2 comprising the amino acid sequence set forth in SEQ ID NO: 4, and a CDR3 comprising the amino acid sequence set forth in SEQ ID NO: 5; the light chain variable region comprises a CDR1 comprising the amino acid sequence set forth in SEQ ID NO: 6, a CDR2 comprising the amino acid sequence set forth in SEQ ID NO: 7, and a CDR3 comprising the amino acid sequence set forth in SEQ ID NO: 27;
(j) the heavy chain variable region comprises a CDR1 comprising the amino acid sequence set forth in SEQ ID NO: 3, a CDR2 comprising the amino acid sequence set forth in SEQ ID NO: 29, and a CDR3 comprising the amino acid sequence set forth in SEQ ID NO: 30; the light chain variable region comprises a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33; or
(k) the heavy chain variable region comprises a CDR1 comprising the amino acid sequence set forth in SEQ ID NO: 36, a CDR2 comprising the amino acid sequence set forth in SEQ ID NO: 37, and a CDR3 comprising the amino acid sequence set forth in SEQ ID NO: 38; the light chain variable region comprises a CDR1 comprising the amino acid sequence set forth in SEQ ID NO: 39, a CDR2 comprising the amino acid sequence set forth in SEQ ID NO: 40, and a CDR3 comprising the amino acid sequence set forth in SEQ ID NO: 41.

In certain embodiments, the heavy chain variable region comprises a CDR1 comprising the amino acid sequence set forth in SEQ ID NO: 3, a CDR2 comprising the amino acid sequence set forth in SEQ ID NO: 4, and a CDR3 comprising the amino acid sequence set forth in SEQ ID NO: 5; the light chain variable region comprises a CDR1 comprising the amino acid sequence set forth in SEQ ID NO: 6, a CDR2 comprising the amino acid sequence set forth in SEQ ID NO: 7, and a CDR3 comprising the amino acid sequence set forth in SEQ ID NO: 8.

In certain embodiments, the antibody or antigen-binding fragment thereof comprises a comprises a heavy chain constant region and/or a light chain constant region. In certain embodiments,
(a) the heavy chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 50; and/or
(b) the light chain constant region comprises an amino acid sequence that is at least about 80%, at least about 85%, at least about 90%, at least about 95%, at least about 96%, at least about 97%, at least about 98%, at least about 99% at least about 100% homologous or identical to the amino acid sequence set forth in SEQ ID NO: 51.

In certain embodiments,
(a) the heavy chain constant region comprises the amino acid sequence set forth in SEQ ID NO: 50; and/or
(b) the light chain constant region comprises the amino acid sequence set forth in SEQ ID NO: 51.

In certain embodiments, the antibody or antigen-binding fragment thereof comprises a human variable region framework region. In certain embodiments, the antibody or antigen-binding fragment thereof is a fully human or an antigen-binding fragment thereof. In certain embodiments, the antibody or antigen-binding fragment thereof is a chimeric antibody or an antigen-binding fragment thereof.

In certain embodiments, the antibody or antigen-binding fragment thereof is a humanized antibody or an antigen-binding fragment thereof. In certain embodiments, the antigen-binding fragment is a Fab, Fab′, F(ab′)₂, variable fragment (Fv), or single chain variable region (scFv). In certain embodiments, the antigen-binding fragment is an scFv.

The presently disclosed subject matter further provides immunoconjugates comprising the antibodies or antigen-binding fragments thereof disclosed herein, linked to a therapeutic agent. In certain embodiments, the therapeutic agent is a drug, a cytotoxin, or a radioactive isotope.

In addition, the presently disclosed subject matter provides multi-specific molecules comprising the antibodies or antigen-binding fragments thereof disclosed herein, linked to one or more functional moieties. In certain embodiments, the one or more functional moieties have a different binding specificity than the antibody or antigen-binding fragment thereof.

The presently disclosed subject matter also provides compositions comprising the antibodies or antigen-binding fragments thereof, the immunoconjugates, or the multi-specific molecules disclosed herein. In certain embodiments, the composition is a pharmaceutical composition that further comprises a pharmaceutically acceptable carrier.

The presently disclosed subject matter further provides nucleic acids encoding the antibodies or antigen-binding fragments thereof disclosed herein. In addition, the presently disclosed subject matter provides vectors, host cells, and lipid nanoparticles including the nucleic acids disclosed herein.

Furthermore, the presently disclosed subject matter provides methods for detecting HPV in a whole cell, a tissue, or a blood sample. In certain embodiments, the methods comprise contacting a cell, tissue or blood sample with the antibody or antigen-binding fragment thereof disclosed herein, wherein the antibody or antigen-binding fragment thereof comprises a detectable label. In certain embodiments, the methods further comprise determining the amount of the labeled antibody or antigen-binding fragment thereof bound to the cell, tissue or blood sample by measuring the amount of detectable label associated with the cell or tissue. In certain embodiments, the amount of bound antibody or antigen-binding fragment thereof indicates the amount of HPV in the cell, tissue or blood sample.

In addition, the presently disclosed subject matter provides antigen-recognizing receptors that target HPV-derived epitopes presented by HLA class I molecules. In certain embodiments, the antigen-recognizing receptor comprises an extracellular antigen-binding domain that binds to an HPV epitope bound to a human major histocompatibility complex (MHC) molecule. In certain embodiments, the extracellular antigen-binding domain comprises an antigen-binding fragment disclosed herein, a transmembrane domain, and an intracellular signaling domain. In certain embodiments, the antigen-recognizing receptor is a chimeric antigen receptor (CAR). In certain embodiments, the intracellular signaling domain comprises a CD3ζ polypeptide. In certain embodiments, the intracellular signaling domain further comprises at least one co-stimulatory signaling region. In certain embodiments, the at least one co-stimulatory signaling region comprises an intracellular signaling domain of a co-stimulatory molecule. In certain embodiments, the co-stimulatory molecule is selected from the group consisting of CD28, 4-1BB, OX40, ICOS, DAP-10, CD27, CD40, NKGD2, CD2, FN14, HVEM, LTBR, CD28H, TNFR1, TNFR2, BAFF-R, BCMA, TACI, TROY, RANK, CD40, CD27, CD30, EDAR, XEDAR, GITR, DR6, NGFR, and combinations thereof. In certain embodiments, the co-stimulatory molecule is CD28 or 4-1BB. In certain embodiments, the antigen-recognizing receptor is a TCR like fusion molecule (HIT) or a T cell receptor fusion construct (TRuC).

The presently disclosed subject matter further provides nucleic acids encoding the antigen-recognizing receptors thereof disclosed herein. In addition, the presently disclosed subject matter provides vectors and lipid nanoparticles including the nucleic acids disclosed herein.

Furthermore, the presently disclosed subject matter provides cells comprising the antigen-recognizing receptors disclosed herein. Also provided are compositions comprising the cells disclosed herein.

The presently disclosed subject matter further provides for methods for treating or ameliorating a disease or disorder in a subject, reducing tumor burden in a subject, treating and/or preventing a tumor in a subject, and/or increasing or lengthening survival of a subject having a tumor. In certain embodiments, the method comprises administering to the subject the antibodies or antigen-binding fragments thereof disclosed herein, the immunoconjugates disclosed herein, the multi-specific molecules disclosed herein, the cells comprising the antigen-recognizing receptor disclosed herein, or the compositions disclosed herein. In certain embodiments, the disease or disorder is a tumor. In certain embodiments, the method reduces the number of the tumor cells, reduces the tumor size, and/or eradicates the tumor in the subject. In certain embodiments, the method reduces or eradicates tumor burden in the subject.

In certain embodiments, the disease or disorder or tumor is a cancer. In certain embodiments, the disease or disorder or tumor is an HPV-associated tumor. In certain embodiments, the disease or disorder or tumor is selected from the group consisting of ovarian cancer, cervical cancer, anal cancer, vaginal cancer, vulvar cancer, penile cancer, head and neck cancer, oropharynx cancer, oropharyngeal squamous cell carcinoma (SCC), anal & rectal SCC, vulvar SCC, vaginal SCC, cervical carcinoma, and penile SCC. In certain embodiments, the disease or disorder or tumor is ovarian cancer. In certain embodiments, the subject is a human.

Finally, the presently disclosed subject matter provides kits for treating or ameliorating a disease or disorder in a subject, reducing tumor burden in a subject, treating and/or preventing a tumor in a subject, and/or increasing or lengthening survival of a subject having a tumor. In certain embodiments, the kit comprises the antibodies or antigen-binding fragments thereof disclosed herein, the immunoconjugates disclosed herein, the multi-specific molecules disclosed herein, the cells comprising the antigen-recognizing receptor disclosed herein, or the compositions disclosed herein.

In certain embodiments, the kit further comprises written instructions.

### 4. BRIEF DESCRIPTION OF THE FIGURES

The following Detailed Description, given by way of example, but not intended to limit the invention to specific embodiments described, may be understood in conjunction with the accompanying drawings.

FIGS. 1A-1D show the binding of the mAbs to the HPV-E7p11-19/HLA-A2 complex. FIG. 1A shows the binding of mAbs to T2 cells pulsed with or without peptides. HPV-E7p11, HPV-E7AAAA or, WT1-RMF peptide at a concentration of 20 ug/ml was pulsed onto T2 cells overnight in serum-free RPMI1640 complete medium. Cells were washed and stained with the mAbs 1B1, 2A5 or 3F8 conjugated to APC at a concentration of 3 ug/ml. A control TCRm mAb specific for WT1-RMF/HLA-A2 complex, ESK1, was used as a negative control for HPV mAb, but positive assay control for RMF/HLA-A2 complex. In parallel, HLA-A2 expression stabilization was determined by staining the cells with anti-HLA-A2 mAb BB7 clone (FIG. 1B). Binding potency of the mAbs was measured by titrating the HPV-E7p11-19 peptide at the indicated concentrations onto T2 cells; the cells were stained with indicated mAbs at 3 ug/ml (FIG. 1C). Mab titration was performed for relative avidity on T2 cells pulsed with HPV-E7p11-19 peptide at 20 ug/ml and stained with the indicated mAbs at concentrations ranging from 3 ug/ml to 0.03 ug/ml) (FIG. 1D). All binding was determined by flow cytometry and indicated by median fluorescence intensity (MFI).

FIGS. 2A-2E show epitope specificity. The target HPV-E7p11-19 peptide sequence or the same sequence substituted with alanine at positions 1, 2, 3, 4, 5, 6, 7, 8, 9 indicated as A1 to A9, respectively, and pulsed onto T2 cells at 20 ug/ml. The HPV sequence with the 4 middle amino acids substituted with alanine (HPV-E7AAAA) as used to gage binding to the center of the sequence as well. The binding of the mAbs 3F8 (FIG. 2A), 1B1 (FIG. 2B), or 2A5 (FIG. 2C) at a concentration of 3 ug/ml was determined by flow cytometric analysis. T2 cells alone, or pulsed with RMF irrelevant peptide were the negative controls. The same cells were simultaneously stained with anti-HLA-A2 mAb, clone BB7.2, to measure the relative binding of the peptides to HLA-A2 molecule (FIG. 2D). The data represent one of two similar experiments. FIG. 2E shows mirror plot of synthetic (bottom) and cell-derived experimental (top) YMLDLQPET peptides. Relative abundance refers to peak areas which are normalized to maximum peak.

FIGS. 3A-3D show correlation of HPV mAb binding and HLA-A2 expression. A panel of cell lines with variable amounts of HLA-A2 on the cell surface were stained with mAbs 1B1, 2A5 and 3F8 at 3 ug/ml and BB7.2 to HLA-A2. HPV mAb binding is shown in Y-axis and HLA-A2 expression in X-Axis. FIG. 3A shows a comparison of all 3 mAb on the same plot. Each mAb alone is plotted: 2A5 (FIG. 3B), 1B1 (FIG. 3C), 3F8 (FIG. 3D). The data are representative for five independent experiments.

FIGS. 4A-4E depict specificity analysis of the 3F8 BiTE. Binding of 3F8 to HPV-E7p11/HLA-A2 complex. T2 cells alone (FIG. 4A) or pulsed with HPV-E7p11-19 (FIG. 4B), or RMF peptide (FIG. 4C) at a concentration of 20 ug/ml overnight. Cells were washed and stained with 3F8 BiTE (3 ug/ml), followed by secondary antibody to His-tag. Binding of 3F8 BiTE to cell surface CD3. Jurkat cells (FIG. 4D) or primary human T cells (FIG. 4E) were stained with 3F8 or control BiTE at a concentration of 3 g/ml, followed by anti-His-PE secondary antibody. A control anti-CD3 antibody was used to confirm the CD3 expression.

FIGS. 5A-5C show cytotoxicity of the 3F8 BiTE. T2 cells alone, or pulsed with HPV-E7p11-19 or RMF control peptide at 50 ug/ml were incubated with human PBMCs at E:T ratio of 20:1 and the 3F8 BiTE (FIG. 5A), or control BiTE (FIG. 5B) at indicated concentrations for 5 hrs and the cytotoxicity was measured by 51Cr-release assay. Similarly, 3F8 BiTE was tested for its cytotoxicity against CasKi (FIG. 5C) without HPV E7-11 peptide pulsing at indicated concentrations. Each data point is the average of triplicate cultures and representative of three similar experiments.

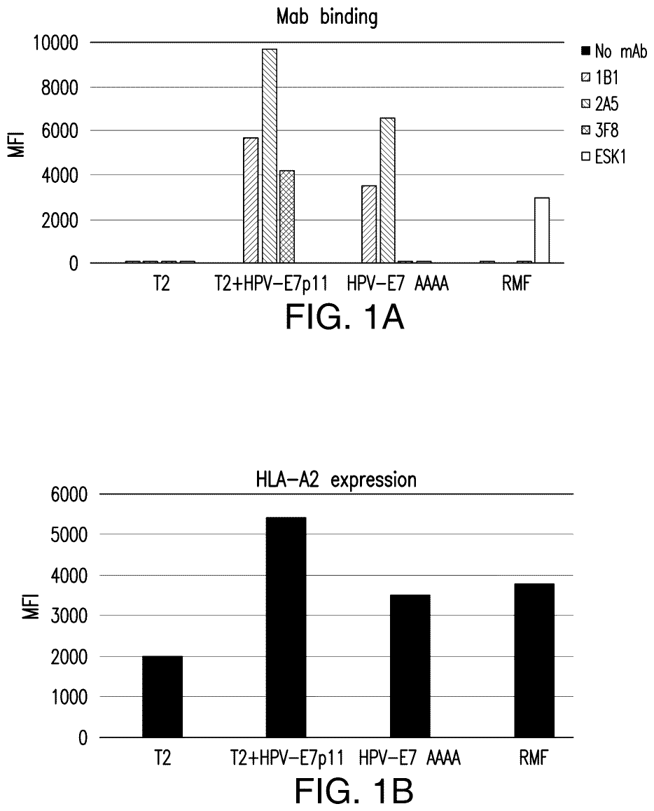

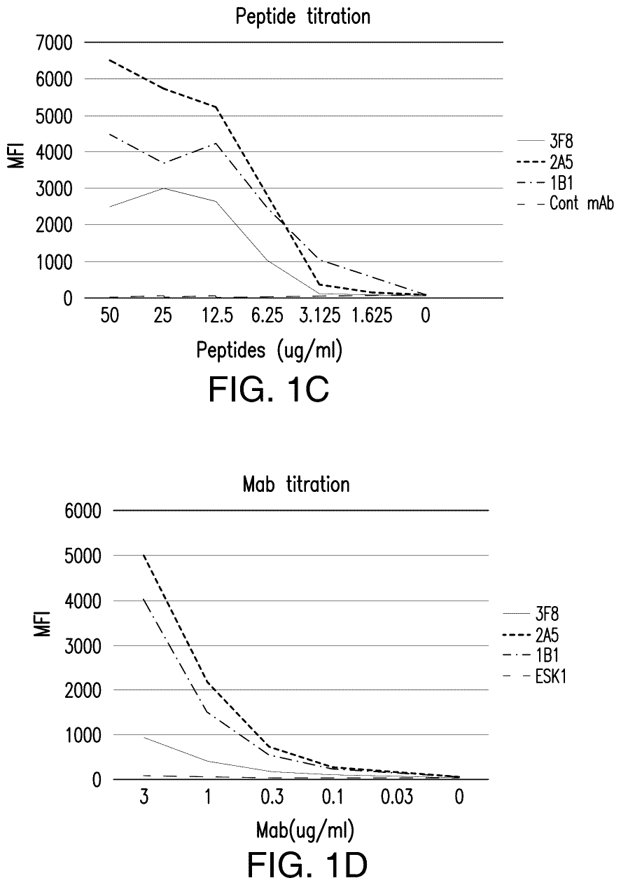

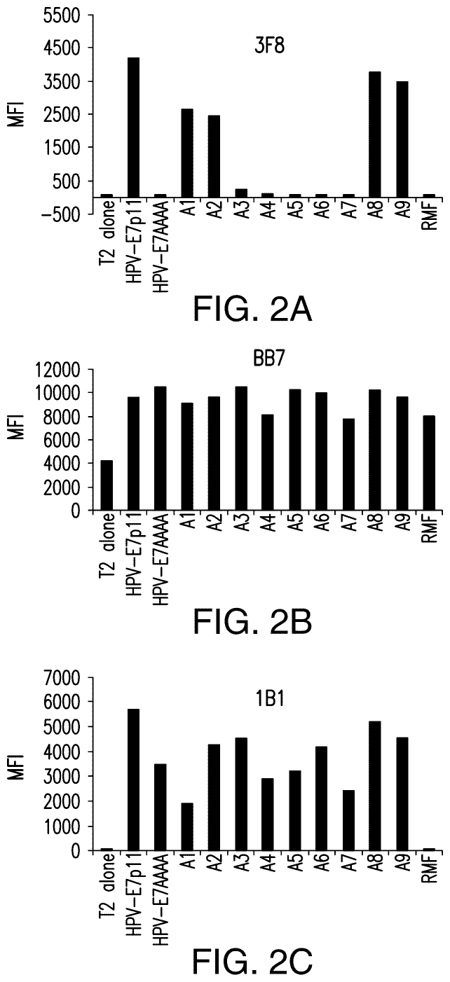

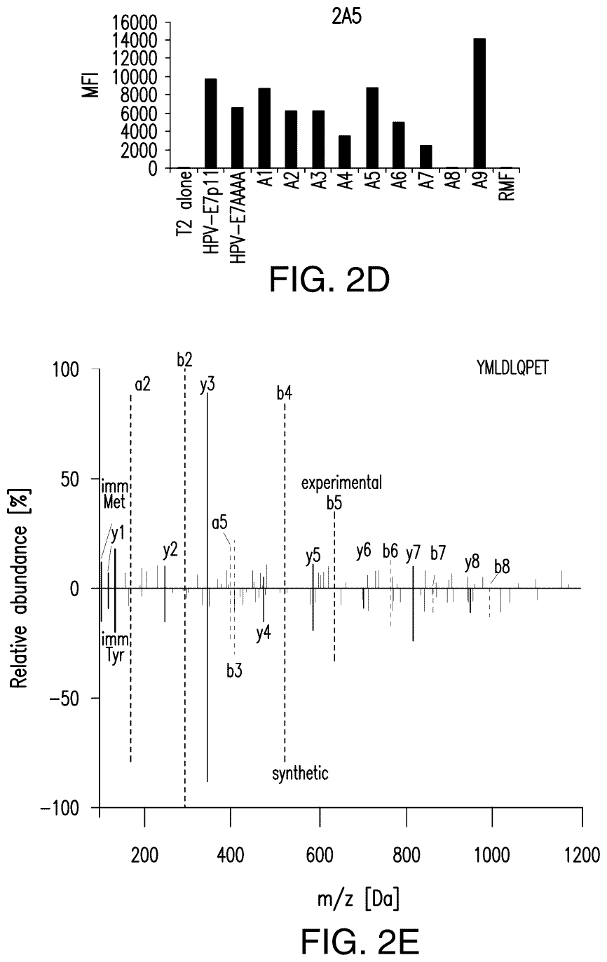

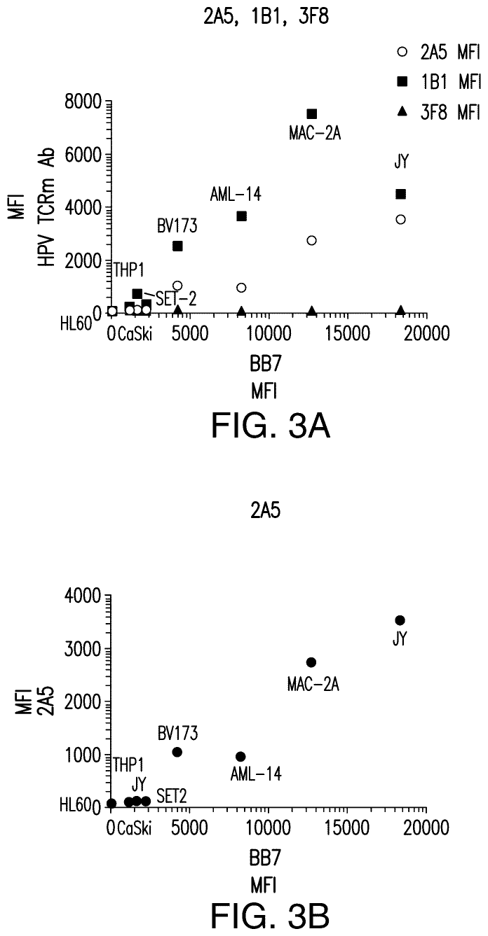

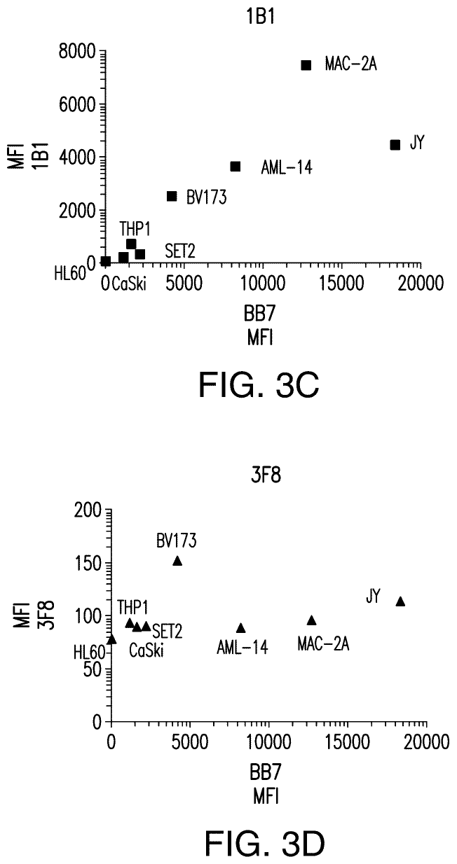

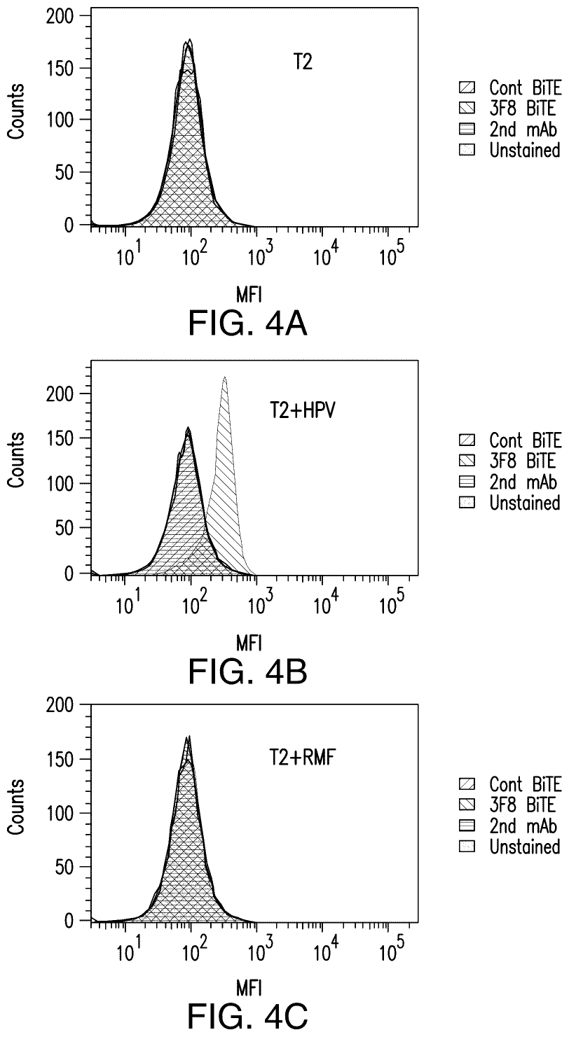

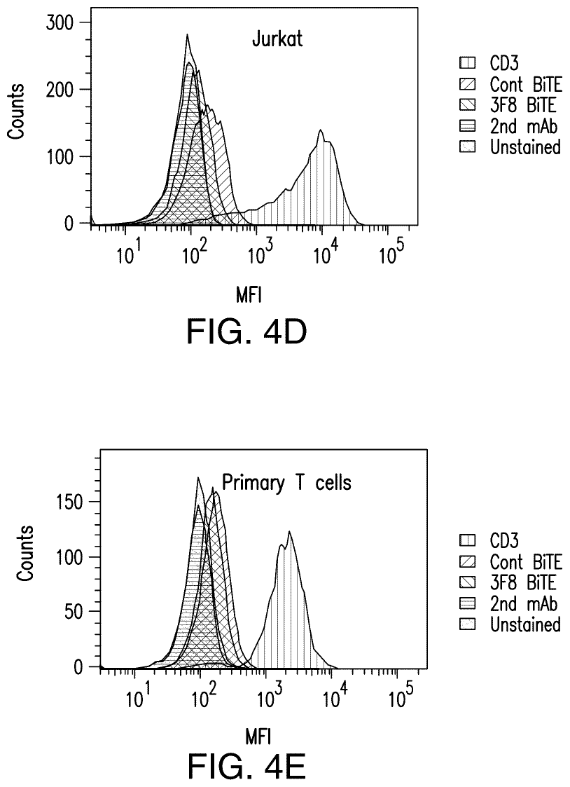

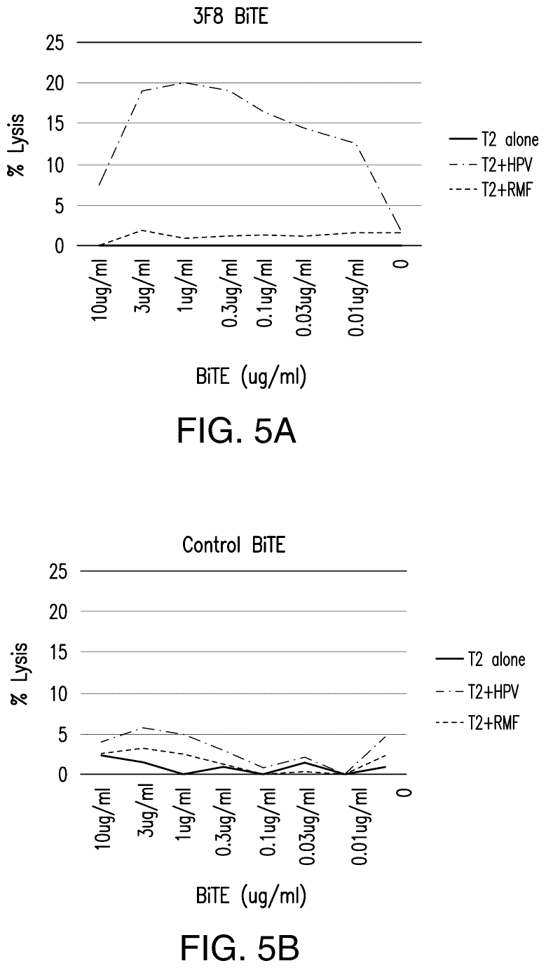

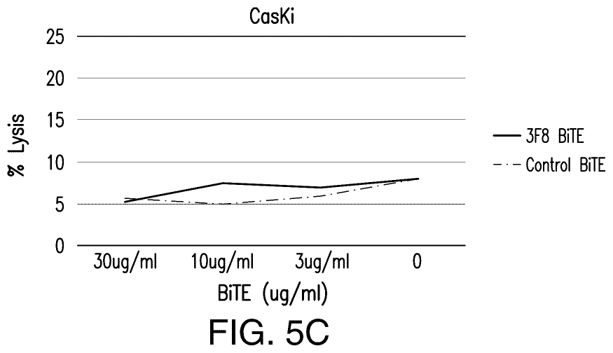

### 5. DETAILED DESCRIPTION OF EMBODIMENTS OF THE INVENTION

The presently disclosed subject matter provides antibodies that mimic TCR recognition of HPV-derived epitopes presented by HLA class I molecules, antigen-recognizing receptors that target HPV-derived epitopes presented by HLA class I molecules, and methods of using such antibodies.

Non-limiting embodiments of the presently disclosed subject matter are described by the present specification and Examples.

For purposes of clarity of disclosure and not by way of limitation, the detailed description is divided into the following subsections:
5.1. Definitions;
5.2. Human papillomavirus (HPV);
5.3. Anti-HPV Antibodies;
5.4. Antigen-Recognizing Receptors;
5.5. Cells comprising the Antibodies or Antigen-Recognizing Receptors;
5.6. Nucleic Acids encoding the Antibodies or Antigen-Recognizing Receptors;
5.7. Pharmaceutical Compositions and Methods of Treatment;
5.8. Diagnostic and Prognostic Methods;
5.9. Kits;
5.10. Exemplary Embodiments.

### 5.1. Definitions

In the description that follows, certain conventions will be followed as regards the usage of terminology. Generally, terms used herein are intended to be interpreted consistently with the meaning of those terms as they are known to those of skill in the art.

“Antibody” and “antibodies” as those terms are known in the art refer to antigen binding proteins of the immune system. The term “antibody” as referred to herein includes whole, full length antibodies having an antigen-binding region, and any fragment thereof in which the “antigen-binding fragment” or “antigen-binding region” is retained, or single chains, for example, single chain variable fragment (scFv), thereof. A naturally occurring “antibody” is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (CH) region. The heavy chain constant region is comprised of three domains, CH1, CH2 and CH3. Each light chain is comprised of a light chain variable region (abbreviated herein as V_{L}) and a light chain constant C_{L} region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1 q) of the classical complement system.

The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the presently disclosed subject matter may include amino acid residues not encoded by human germline immunoglobulin sequences (e.g., mutations introduced by random or site-specific mutagenesis in vitro or by somatic mutation in vivo).

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the presently disclosed subject matter may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “recombinant human antibody”, as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

The term “humanized antibody” is intended to refer to antibodies in which CDR sequences derived from the germline of another mammalian species, such as a mouse, have been grafted onto human framework sequences. Additional framework region modifications may be made within the human framework sequences.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

As used herein, an antibody that “specifically binds to HPV/MHC complex” is intended to refer to an antibody that recognizes and binds to an HPV/MHC complex (e.g., an HPV/HLA complex, more specifically, a HPV/HLA class I complex, more specifically, a HPV/HLA-A complex, more specifically, a HPV/HLA-A2 complex, or more specifically, a HPV/HLA-A*02:01 complex) with a dissociation constant (K_{D}) of about 1×10⁻⁸ M or less, about 5×10⁻⁹ M or less, about 1×10⁻⁹ M or less, about 5×10⁻¹ M or less, about 1×10⁻¹ M or less, about 5×10⁻¹¹ M or less, or about 1×10⁻¹¹ M or less.

An “antibody that competes for binding” or “antibody that cross-competes for binding” with a reference antibody for binding to an antigen/MHC complex, e.g., HPV/MHC complex, refers to an antibody that blocks binding of the reference antibody to the antigen/MHC (e.g., an HPV/HLA complex, more specifically, a HPV/HLA class I complex, more specifically, a HPV/HLA-A complex, more specifically, a HPV/HLA-A2 complex, or more specifically, a HPV/HLA-A*02:01 complex) in a competition assay by 50% or more, and conversely, the reference antibody blocks binding of the antibody to the antigen/MHC complex (e.g., an HPV/HLA complex, more specifically, a HPV/HLA class I complex, more specifically, a HPV/HLA-A complex, more specifically, a HPV/HLA-A2 complex, or more specifically, a HPV/HLA-A*02:01 complex) in a competition assay by 50% or more. An exemplary competition assay is described in “Antibodies”, Harlow and Lane (Cold Spring Harbor Press, Cold Spring Harbor, NY).

As used herein, “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen” (e.g., an HPV/HLA complex, more specifically, a HPV/HLA class I complex, more specifically, a HPV/HLA-A complex, more specifically, a HPV/HLA-A2 complex, or more specifically, a HPV/HLA-A*02:01 complex).

The term “antigen-binding fragment” or “antigen-binding region” of an antibody, as used herein, refers to that region or fragment of the antibody that binds to the antigen and which confers antigen specificity to the antibody; fragments of antigen-binding proteins, for example, antibodies includes one or more fragments of an antibody that retain the ability to specifically bind to an antigen (e.g., an HPV/HLA complex, more specifically, a HPV/HLA class I complex, more specifically, a HPV/HLA-A complex, more specifically, a HPV/HLA-A2 complex, or more specifically, a HPV/HLA-A*02:01 complex). It has been shown that the antigen-binding function of an antibody can be performed by fragments of a full-length antibody. Examples of antigen-binding fragments encompassed within the term “antibody fragments” of an antibody include a Fab fragment, a monovalent fragment consisting of the V_{L}, V_{H}, C_{L} and CH1 domains; a F(ab)₂ fragment, a bivalent fragment comprising two Fab fragments linked by a disulfide bridge at the hinge region; a Fd fragment consisting of the V_{H} and CH1 domains; a Fv fragment consisting of the V_{L} and V_{H} domains of a single arm of an antibody; a dAb fragment (Ward et al., Nature 1989; 341:544-546), which consists of a V_{H} domain; and an isolated complementarity determining region (CDR).

Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules. These are known as single chain Fv (scFv); see e.g., Bird et al., Science (1988); 242:423-426; and Huston et al., Proc Natl Acad Sci (1998); 85:5879-5883. These antibody fragments are obtained using conventional techniques known to those of skill in the art, and the fragments are screened for utility in the same manner as are intact antibodies.

An “antibody” or “antigen-binding protein” is one which has been identified and separated and/or recovered from a component of its natural environment. “Synthetic antibodies” or “recombinant antibodies” are generally generated using recombinant technology or using peptide synthetic techniques known to those of skill in the art.

As used herein, the terms “TCR mimic” or “TCRm” refer to antibodies targeting peptide-MHC complexes. TCR mimics are structurally identical with traditional antibodies and recognize a composite antigen comprised of a variable linear sequence (typically 9-10 amino acids in length) buried within an MHC molecule that is largely invariant because they bind to a peptide-MHC complex. Additional information on TCR mimics can be found in U.S. Pat. No. 11,168,150 and in Chang et al., Expert opinion on biological therapy 16.8 (2016): 979-987, the contents of each of which are incorporated by reference in their entireties.

As used herein, the term “single-chain variable fragment” or “scFv” is a fusion protein of the variable regions of the heavy (V_{H}) and light chains (V_{L}) of an immunoglobulin (e.g., mouse or human) covalently linked to form a V_{H}::V_{L} heterodimer. The heavy (V_{H}) and light chains (V_{L}) are either joined directly or joined by a peptide-encoding linker (e.g., 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. The linker can link the heavy chain variable region and the light chain variable region of the extracellular antigen-binding domain.

Non-limiting examples of linkers are disclosed in Shen et al., Anal Chem (2008); 80(6):1910-1917 and WO 2014/087010, the contents of which are hereby incorporated by reference in their entireties. In certain embodiments, the linker is a G4S linker. In certain embodiments, the linker comprises or consists of the amino acid sequence set forth in SEQ ID NO: 44, which is provided below:

[SEQ ID NO: 44]
GGGGSGGGGSGGGSGGGGS

In certain embodiments, the linker comprises or consists of the amino acid sequence set forth in SEQ ID NO: 45, which is provided below:

[SEQ ID NO: 45]
GGGGSGGGGSGGGGS

In certain embodiments, the linker comprises or consists of the amino acid sequence set forth in SEQ ID NO: 46, which is provided below:

[SEQ ID NO: 46]
GGGGSGGGGSGGGGSGGGSGGGGS

In certain embodiments, the linker comprises or consists of the amino acid sequence set forth in SEQ ID NO: 47, which is provided below:

[SEQ ID NO: 47]
GGGGSGGGGSGGGGSGGGGSGGGSGGGGS

In certain embodiments, the linker comprises or consists of the amino acid sequence set forth in SEQ ID NO: 48, which is provided below:

[SEQ ID NO: 48]
GGGGS

In certain embodiments, the linker comprises or consists of the amino acid sequence set forth in SEQ ID NO: 49, which is provided below:

[SEQ ID NO: 49]
SGGGGS

As used herein, “F(ab)” refers to a fragment of an antibody structure that binds to an antigen but is monovalent and does not have a Fc portion, for example, an antibody digested by the enzyme papain yields two F(ab) fragments and an Fc fragment (e.g., a heavy (H) chain constant region; Fc region that does not bind to an antigen).

As used herein, “F(ab′)₂” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)₂” fragment can be split into two individual Fab′ fragments.

“CDRs” are defined as the complementarity determining region amino acid sequences of an antibody which are the hypervariable regions of immunoglobulin heavy and light chains. See, e. g., Kabat et al., Sequences of Proteins of Immunological Interest, 4th U. S. Department of Health and Human Services, National Institutes of Health (1987), or IMGT numbering system (Lefranc, The Immunologist (1999); 7:132-136; Lefranc et al., Dev. Comp. Immunol. (2003); 27:55-77). The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise three heavy chain and three light chain CDRs or CDR regions in the variable region. CDRs provide the majority of contact residues for the binding of the antibody to the antigen or epitope region. In certain embodiments, the CDRs are identified according to the IMGT system. In certain embodiments, the CDRs are identified using the IMGT numbering system accessible at http://www.imgt.org/IMGT_vquest/input.

The terms “isolated” denotes a degree of separation from original source or surroundings.

An “isolated antibody” is one which has been separated from a component of its natural environment. In certain embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr (2007); B 848:79-87.

An “isolated nucleic acid” refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

An “isolated nucleic acid encoding an antibody” (including references to a specific antibody, e.g. an anti-HPV antibody disclosed herein) refers to one or more nucleic acid molecules encoding antibody heavy and light chains (or fragments thereof), including such nucleic acid molecule(s) in a single vector separate vectors, and such nucleic acid molecule(s) present at one or more locations in a host cell.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

An “immunoconjugate” is an antibody conjugated to one or more heterologous molecule(s), including, but not limited to, a cytotoxic agent.

An “effective amount” (or, “therapeutically effective amount”) is an amount sufficient to effect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition, and the form and effective concentration of the cells administered.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, guinea pigs, rabbits, dogs, cats, sheep, pigs, goats, cattle, horses, and non-human primates such as apes and monkeys.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In certain embodiments, antibodies of the presently disclosed subject matter are used to delay development of a disease or to slow the progression of a disease, e.g., a tumor, e.g., a tumor associated with HPV.

The terms “comprises”, “comprising”, and are intended to have the broad meaning ascribed to them in U.S. Patent Law and can mean “includes”, “including” and the like.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As described herein, any concentration range, percentage range, ratio range or integer range is to be understood to include the value of any integer within the recited range and, when appropriate, fractions thereof (such as one tenth and one hundredth of an integer), unless otherwise indicated.

Other aspects of the presently disclosed subject matter are described in the following disclosure and are within the ambit of the presently disclosed subject matter.

### 5.2. Human Papillomavirus (HPV)

Human papillomavirus (HPV) is a small, non-enveloped deoxyribonucleic acid (DNA) virus that infects skin or mucosal cells. The circular, double-stranded viral genome is approximately 8-kb in length. The genome encodes for 6 early proteins responsible for virus replication and 2 late proteins, L1 and L2, which are the viral structural proteins. At least 13 of more than 100 known HPV genotypes can cause cancer of the cervix and are associated with other anogenital cancers and cancers of the head and neck. The two most common “high-risk” genotypes (HPV 16 and 18) cause approximately 70% of all cervical cancers (Zur Hausen, Nature reviews cancer 2.5 (2002): 342-350). Two transforming proteins encoded by HPV are E6 and E7. E6 facilitates the degradation of p53 through its association with an accessory protein, E6-AP, a component of the ubiquitin proteolytic pathway (Huibregtse et al., The EMBO journal 10.13 (1991): 4129-4135). E7 binds to Rb, leading to the altered activities of these cell cycle regulators (Aarthy et al. Gene 658 (2018): 159-177).

In certain embodiments, the presently disclosed anti-HPV antibodies or antigen-binding fragments thereof bind to an HPV/HLA complex. In certain embodiments, the HPV/HLA complex is an HPV/HLA class I complex. In certain embodiments, the HPV/HLA complex is an HPV/HLA-A complex. In certain embodiments, the HPV/HLA complex is an HPV/HLA-A2 complex. In certain embodiments, the HPV/HLA complex is an HPV/HLA-A*02:01 complex.

In certain embodiments, the HPV/HLA complex comprises an HPV peptide. In certain embodiments, the HPV peptide is an E7 epitope. In certain embodiments, the E7 epitope comprises or consists of a fragment of the amino acid sequence with a UniProt Reference No: P03129 (SEQ ID NO: 2). In certain embodiments, the E7 epitope is between about 8 amino acid and about 12 amino acids in length. In certain embodiments, the E7 epitope is about 9 amino acid in length. In certain embodiments, the E7 epitope comprises or consists of the amino acid sequence set forth in SEQ ID NO: 1. SEQ ID NO: 1 and SEQ ID NO: 2 are provided below.

[SEQ ID NO: 1]
YMLDLQPET
[SEQ ID NO: 2]
MHGDTPTLHEYMLDLQPETTDLYCYEQLNDSSEEEDEIDGPAGQAEPDR
AHYNIVTFCCKCDSTLRLCVQSTHVDIRTLEDLLMGTLGIVCPICSQKP

### 5.3. Anti-HPV Antibodies

The presently disclosed subject matter employs an approach to obtaining therapeutic antibodies to any protein, including those proteins that are inaccessible because they are not expressed on the cell surface, e.g., HPV E7.

In order to target antigens derived from intracellular or nuclear proteins, development of a therapeutic antibody an uncommon approach is required. This approach is to generate recombinant antibodies (e.g., monoclonal Abs (mAb)) that recognize the peptide/MHC complex expressed on the cell surface, with the same specificity as a T-cell receptor (TCR). These antibodies are TCR mimics and share functional homology with TCRs regarding target recognition. TCR mimics confer higher affinity and capabilities of arming with potent cytotoxic agents that antibodies feature. TCR mimics can be generated, for example and without any limitation, by conventional hybridoma techniques or by in vitro antibody library techniques known to those of skill in the art, to produce human, humanized or chimeric antibodies.

The presently disclosed subject matter involves the development of a TCR mimic that recognizes, for example, the E7 epitope/HLA-A2 complex for cancer therapy. Therefore, the presently disclosed subject matter provides antibodies (e.g., monoclonal antibodies) specifically binding to HPV.

The antibodies of the presently disclosed subject matter are characterized by particular functional features or properties of the antibodies. For example, the antibodies bind specifically to HPV (e.g., bind an HPV/HLA complex, more specifically, a HPV/HLA class I complex, more specifically, a HPV/HLA-A complex, more specifically, a HPV/HLA-A2 complex, or more specifically, a HPV/HLA-A*02:01 complex).

In certain embodiments, the presently disclosed antibody or antigen-binding fragment binds to HPV (e.g., an HPV/HLA complex, more specifically, a HPV/HLA class I complex, more specifically, a HPV/HLA-A complex, more specifically, a HPV/HLA-A2 complex, or more specifically, a HPV/HLA-A*02:01 complex) with a binding affinity, for example with a dissociation constant (K_{D}) of about 5×10⁻⁶ M or less, e.g., about 1×10⁻⁶ M or less, 5×10⁻⁷ M or less, 1×10⁻⁷ M or less, 5×10⁻⁸ M or less, 1×10⁻⁸ M or less, about 5×10⁻⁹ M or less, about 1×10⁻⁹ M or less, about 5×10⁻¹⁰ M or less, about 1×10⁻¹⁰ M or less, or about 1×10⁻¹¹ M or less. In certain embodiments, the presently disclosed anti-HPV antibody or antigen-binding fragment thereof binds to HPV (e.g., an HPV/HLA complex, more specifically, a HPV/HLA class I complex, more specifically, a HPV/HLA-A complex, more specifically, a HPV/HLA-A2 complex, or more specifically, a HPV/HLA-A*02:01 complex) with a dissociation constant (K_{D}) of between about 1×10⁻⁶ M and about 5×10⁻⁶ M, e.g., about 2×10⁻⁶ M, e.g., 1.8×10⁻⁶ M. In certain embodiments, the presently disclosed anti-HPV antibody or antigen-binding fragment thereof binds to HPV (e.g., an HPV/HLA complex, more specifically, a HPV/HLA class I complex, more specifically, a HPV/HLA-A complex, more specifically, a HPV/HLA-A2 complex, or more specifically, a HPV/HLA-A*02:01 complex) with a dissociation constant (K_{D}) of between about 1×10⁻⁷ M and about 1×10⁻⁶ M. In certain embodiments, the presently disclosed anti-HPV antibody or antigen-binding fragment thereof binds to HPV (e.g., an HPV/HLA complex, more specifically, a HPV/HLA class I complex, more specifically, a HPV/HLA-A complex, more specifically, a HPV/HLA-A2 complex, or more specifically, a HPV/HLA-A*02:01 complex) with a dissociation constant (K_{D}) of about 2×10⁻⁷ M. In certain embodiments, the presently disclosed anti-HPV antibody or antigen-binding fragment thereof binds to HPV (e.g., an HPV/HLA complex, more specifically, a HPV/HLA class I complex, more specifically, a HPV/HLA-A complex, more specifically, a HPV/HLA-A2 complex, or more specifically, a HPV/HLA-A*02:01 complex) with a dissociation constant (K_{D}) of between about 1×108 M and about 5×10⁻⁸ M. In certain embodiments, the presently disclosed anti-HPV antibody or antigen-binding fragment thereof binds to HPV (e.g., an HPV/HLA complex, more specifically, a HPV/HLA class I complex, more specifically, a HPV/HLA-A complex, more specifically, a HPV/HLA-A2 complex, or more specifically, a HPV/HLA-A*02:01 complex) with a dissociation constant (K_{D}) of about 3×10⁻⁸ M.

In certain embodiments, the presently disclosed antibody or antigen-binding fragment binds to a cell expressing HPV (e.g., an ovarian cell expressing an HPV/MHC complex) with a Half maximal Effective Concentration (EC₅₀) value of from about 1 nM to about 50 nM, from about 5 nM to about 50 nM, from about 10 nM to about 50 nM, from about 20 nM to about 50 nM, from about 30 nM to about 50 nM, from about 40 nM to about 50 nM, or greater than about 50 nM. In certain embodiments, the presently disclosed antibody or antigen-binding fragment binds to a cell expressing HPV (e.g., an ovarian cell expressing an HPV/MHC complex) with an EC₅₀ value from about 1 nM to about 5 nM. In certain embodiments, the presently disclosed antibody or antigen-binding fragment binds to a cell expressing HPV (e.g., an ovarian cell expressing an HPV/MHC complex) with an EC₅₀ value from about 5 nM to about 10 nM. In certain embodiments, the presently disclosed antibody or antigen-binding fragment binds to a cell expressing HPV (e.g., an ovarian cell expressing an HPV/MHC complex) with an EC₅₀ value from about 40 nM to about 50 nM.

In certain embodiments, the presently disclosed antibody or antigen-binding fragment binds to a cell expressing HPV (e.g., an ovarian cell expressing an HPV/MHC complex) with a Half maximal Effective Concentration (EC₅₀) value of from about 1 ng/ml to about 500 ng/ml, from about 10 ng/ml to about 500 ng/ml, from about 20 ng/ml to about 500 ng/ml, from about 50 ng/ml to about 500 ng/ml, from about 200 ng/ml to about 500 ng/ml, from about 300 nM to about 500 ng/ml, or greater than about 500 ng/ml. In certain embodiments, the presently disclosed antibody or antigen-binding fragment binds to a cell expressing HPV (e.g., an ovarian cell expressing an HPV/MHC complex) with an EC₅₀ value from about 10 ng/ml to about 30 ng/ml. In certain embodiments, the presently disclosed antibody or antigen-binding fragment binds to a cell expressing HPV (e.g., an ovarian cell expressing an HPV/MHC complex) with an EC₅₀ value of about 20 ng/ml. In certain embodiments, the presently disclosed antibody or antigen-binding fragment binds to a cell expressing HPV (e.g., an ovarian cell expressing an HPV/MHC complex) with an EC₅₀ value of about 19.2 ng/ml. In certain embodiments, the presently disclosed antibody or antigen-binding fragment binds to a cell expressing HPV (e.g., an ovarian cell expressing an HPV/MHC complex) with an EC₅₀ value of about 21 ng/ml. In certain embodiments, the presently disclosed antibody or antigen-binding fragment binds to a cell expressing HPV (e.g., an ovarian cell expressing an HPV/MHC complex) with an EC₅₀ value of about 26 ng/ml. In certain embodiments, the presently disclosed antibody or antigen-binding fragment binds to a cell expressing HPV (e.g., an ovarian cell expressing an HPV/MHC complex) with an EC₅₀ value from about 50 ng/ml to about 100 ng/ml. In certain embodiments, the presently disclosed antibody or antigen-binding fragment binds to a cell expressing HPV (e.g., an ovarian cell expressing an HPV/MHC complex) with an EC₅₀ value of about 50 ng/ml. In certain embodiments, the presently disclosed antibody or antigen-binding fragment binds to a cell expressing HPV (e.g., an ovarian cell expressing an HPV/MHC complex) with an EC₅₀ value of about 52 ng/ml. In certain embodiments, the presently disclosed antibody or antigen-binding fragment binds to a cell expressing HPV (e.g., an ovarian cell expressing an HPV/MHC complex) with an EC₅₀ value of about 60 ng/ml. In certain embodiments, the presently disclosed antibody or antigen-binding fragment binds to a cell expressing HPV (e.g., an ovarian cell expressing an HPV/MHC complex) with an EC₅₀ value of about 62 ng/ml. In certain embodiments, the presently disclosed antibody or antigen-binding fragment binds to a cell expressing HPV (e.g., an ovarian cell expressing an HPV/MHC complex) with an EC₅₀ value from about 200 ng/ml to about 300 ng/ml. In certain embodiments, the presently disclosed antibody or antigen-binding fragment binds to a cell expressing HPV (e.g., an ovarian cell expressing an HPV/MHC complex) with an EC₅₀ value of about 215 ng/ml. In certain embodiments, the presently disclosed antibody or antigen-binding fragment binds to a cell expressing HPV (e.g., an ovarian cell expressing an HPV/MHC complex) with an EC₅₀ value of about 217 ng/ml. In certain embodiments, the presently disclosed antibody or antigen-binding fragment binds to a cell expressing HPV (e.g., an ovarian cell expressing an HPV/MHC complex) with an EC₅₀ value from about 300 ng/ml to about 400 ng/ml. In certain embodiments, the presently disclosed antibody or antigen-binding fragment binds to a cell expressing HPV (e.g., an ovarian cell expressing an HPV/MHC complex) with an EC₅₀ value of about 375 ng/ml. In certain embodiments, the presently disclosed antibody or antigen-binding fragment binds to a cell expressing HPV (e.g., an ovarian cell expressing an HPV/MHC complex) with an EC₅₀ value of about 374 ng/ml.

In certain embodiments, the presently disclosed antibody or antigen-binding fragment binds to an HPV polypeptide associated with an HLA-A*02 superfamily (e.g., in an HLA-A*02 superfamily dependent manner). In certain embodiments, the HLA-A*02 superfamily member is selected from the group consisting of HLA-A*02:01, HLA-A*02:02, HLA-A*02:03, HLA-A*02:05, and HLA-A*02:06. In certain embodiments, the presently disclosed antibody or antigen-binding fragment binds to an HPV polypeptide associated with an HLA-A*02:01 molecule.

The heavy and light chains of a presently disclosed antibody or antigen-binding fragment can be full-length (e.g., an antibody can include at least one (e.g., one or two) complete heavy chains, and at least one (e.g., one or two) complete light chains) or can include an antigen-binding fragment (a Fab, F(ab′)₂, Fv or a single chain Fv fragment (“scFv”)). In certain embodiments, the antibody heavy chain constant region is chosen from, e.g., IgG1, IgG2, IgG3, IgG4, IgM, IgA1, IgA2, IgD, and IgE, particularly chosen from, e.g., IgG1, IgG2, IgG3, and IgG4. In certain embodiments, the immunoglobulin isotype is IgG1 (e.g., human IgG1). The choice of antibody isotype can depend on the immune effector function that the antibody is designed to elicit. In certain embodiments, the antibody light chain constant region is chosen from, e.g., kappa or lambda, particularly kappa.

In constructing a recombinant immunoglobulin, appropriate amino acid sequences for constant regions of various immunoglobulin isotypes and methods for the production of a wide array of antibodies are known to those of skill in the art.

5.3.1. Single-Chain Variable Fragments (scFvs)

In certain embodiments, the presently disclosed subject matter includes antibodies or antigen-binding fragments thereof that have the scFv sequence fused to one or more constant domains to form an antibody with an Fc region of a human immunoglobulin to yield a bivalent protein, increasing the overall avidity and stability of the antibody. In addition, the Fc portion allows the direct conjugation of other molecules, including but not limited to fluorescent dyes, cytotoxins, radioisotopes etc. to the antibody for example, for use in antigen quantitation studies, to immobilize the antibody for affinity measurements, for targeted delivery of a therapeutic agent, to test for Fc-mediated cytotoxicity using immune effector cells and many other applications.

The results presented here highlight the specificity, sensitivity and utility of the presently disclosed antibodies or antigen-binding fragments in targeting an HPV polypeptide (e.g., an HPV/HLA complex, more specifically, a HPV/HLA class I complex, more specifically, a HPV/HLA-A complex, more specifically, a HPV/HLA-A2 complex, or more specifically, a HPV/HLA-A*02:01 complex).

In certain embodiments, the anti-HPV scFv is an scFv-Fc fusion protein or a full-length human IgG with V_{H} and V_{L} regions or CDRs selected from Table 1. In certain embodiments, the anti-HPV scFv comprises a heavy chain variable region (V_{H}) comprising the amino acid sequence set forth in SEQ ID NO: 9. In certain embodiments, the anti-HPV scFv comprises a light chain variable region (V_{L}) comprising the amino acid sequence set forth in SEQ ID NO: 10. An exemplary nucleotide sequence encoding the amino acid sequence of SEQ ID NO: 9 is set forth in SEQ ID NO: 52. An exemplary nucleotide sequence encoding the amino acid sequence of SEQ ID NO: 10 is set forth in SEQ ID NO: 53. SEQ ID NO: 8, 10, 52, and 53 are provided in Table 1. In certain embodiments, the scFv is designated as “3F8”.

In certain embodiments, the anti-HPV scFv comprises a V_{H} Comprising the amino acid sequence set forth in SEQ ID NO: 9 and a V_{L} comprising the amino acid sequence set forth in SEQ ID NO: 10.

In certain embodiments, the anti-HPV scFv comprises a V_{H} that comprises a CDR1 comprising the amino acid sequence set forth in SEQ ID NO: 3 or a conservative modification thereof, a CDR2 comprising the amino acid sequence set forth in SEQ ID NO: 4 or a conservative modification thereof, and a CDR3 comprising the amino acid sequence set forth in SEQ ID NO: 5 or a conservative modification thereof. SEQ ID NOs: 3-5 are provided in Table 1.

In certain embodiments, the anti-HPV scFv comprises a V_{L} that comprises a CDR1 comprising the amino acid sequence set forth in SEQ ID NO: 6 or a conservative modification thereof, a CDR2 comprising the amino acid sequence set forth in SEQ ID NO: 7 or a conservative modification thereof, and a CDR3 comprising the amino acid sequence set forth in SEQ ID NO: 8 or a conservative modification thereof. SEQ ID NOs: 6-8 are provided in Table 1.

In certain embodiments, the anti-HPV scFv comprises a V_{H} that comprises a CDR1 comprising the amino acid sequence set forth in SEQ ID NO: 3 or a conservative modification thereof, a CDR2 comprising the amino acid sequence set forth in SEQ ID NO: 4 or a conservative modification thereof, and a CDR3 comprising the amino acid sequence set forth in SEQ ID NO: 5 or a conservative modification thereof; and a V_{L} that comprises a CDR1 comprising the amino acid sequence set forth in SEQ ID NO: 6 or a conservative modification thereof, a CDR2 comprising the amino acid sequence set forth in SEQ ID NO: 7 or a conservative modification, and a CDR3 comprising the amino acid sequence set forth in SEQ ID NO: 8 or a conservative modification thereof.

In certain embodiments, the anti-HPV scFv comprises a V_{H} that comprises a CDR1 comprising the amino acid sequence set forth in SEQ ID NO: 3, a CDR2 comprising the amino acid sequence set forth in SEQ ID NO: 4, and a CDR3 comprising the amino acid sequence set forth in SEQ ID NO: 5; and a V_{L} that comprises a CDR1 comprising the amino acid sequence set forth in SEQ ID NO: 6, a CDR2 comprising the amino acid sequence set forth in SEQ ID NO: 7, and a CDR3 comprising the amino acid sequence set forth in SEQ ID NO: 8.

In certain embodiments, the anti-HPV scFv comprises a V_{H} comprising the amino acid sequence set forth in SEQ ID NO: 9, and a V_{L} comprising the amino acid sequence set forth in SEQ ID NO: 10. In certain embodiments, the V_{H} and V_{L} are linked via a linker. In certain embodiments, the linker comprises or consists of the amino acid sequence set forth in SEQ ID NO: 44, SEQ ID NO: 45, SEQ ID NO: 46, SEQ ID NO: 47, SEQ ID NO: 48, or SEQ ID NO: 49.

In certain embodiments, the variable regions are linked one after another such that a heavy chain variable region (V_{H}) is position at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 1

| CDRs | 1 | 2 | 3 |
|---|---|---|---|
| V_{H} | GFTFSDYY [SEQ ID | IYGGGGST [SEQ ID | AKDRWGYSSGWHNFDF |
| | NO: 3] | NO: 4] | [SEQ ID NO: 5] |
| V_{L} | QSVLRSGNNKND | WAS | QQSSSEPIT |
| | [SEQ ID NO: 6] | [SEQ ID NO: 7] | [SEQ ID NO: 8] |

| | |
|---|---|
| Full V_{H} | EVQLLESGGGLVQPGGSLRLSCAASGFTFSDYYMSWVRQAPGKGLEWVSGIYGGGGSTR |
| | YADSVKGRFTISRDNSKNTLYLQMNSLRAEDTAVYYCAKDRWGYSSGWHNFDFWGQGTL |
| | VTVSS [SEQ ID NO: 9] |
| Full V_{L} | DIVMTQSPDSLAVSLGERATINCKSSQSVLRSGNNKNDLAWYQQKPGQPPKLLI |
| | YWASTRESGVPDRFSGSGSGTDFTLTISSLQAEDVAVYYCQQSSSEPITFGQGT |
| | KVEIK [SEQ ID NO: 10] |
| DNA for | GAAGTGCAGCTGCTGGAATCTGGCGGAGGACTGGTTCAACCTGGCGGCTCTCTG |
| Full V_{H} | AGACTGTCTTGTGCCGCCAGCGGCTTCACCTTCAGCGACTACTACATGAGCTGG |
| | GTCCGACAGGCCCCTGGCAAAGGACTTGAATGGGTGTCCGGCATCTATGGCGGC |
| | GGAGGCTCTACAAGATACGCCGACTCTGTGAAGGGCAGATTCACCATCAGCCGG |
| | GACAACAGCAAGAACACCCTGTACCTGCAGATGAACAGCCTGAGAGCCGAGGAC |
| | ACCGCCGTGTACTACTGCGCCAAAGACAGATGGGGCTACAGCAGCGGCTGGCAC |
| | AACTTCGATTTTTGGGGCCAGGGCACCCTGGTCACCGTTTCTTCT [SEQ ID |
| | NO: 52] |
| DNA for | GACATCGTGATGACACAGAGCCCTGATAGCCTGGCCGTGTCTCTGGGAGAGAGA |
| Full V_{L} | GCCACCATCAACTGCAAGAGCAGCCAGAGCGTGCTGCGGAGCGGCAACAACAAG |
| | AATGACCTGGCCTGGTATCAGCAGAAGCCCGGCCAACCTCCTAAGCTGCTGATC |
| | TACTGGGCCAGCACCAGAGAAAGCGGCGTGCCCGATAGATTTTCTGGCAGCGGC |
| | TCTGGCACCGACTTCACCCTGACAATTAGCTCCCTGCAGGCCGAGGATGTGGCC |
| | GTGTACTACTGTCAGCAGAGCAGCAGCGAGCCCATCACATTTGGCCAGGGCACC |
| | AAGGTGGAAATCAAG [SEQ ID NO: 53] |

In certain embodiments, the anti-HPV scFv is an scFv-Fc fusion protein or a full-length human IgG with V_{H} and V_{L} regions or CDRs selected from Table 2. In certain embodiments, the anti-HPV scFv comprises a V_{H} comprising the amino acid sequence set forth in SEQ ID NO: 9. An exemplary nucleotide sequence encoding the amino acid sequence of SEQ ID NO: 9 is set forth in SEQ ID NO: 52. In certain embodiments, the anti-HPV scFv comprises a V_{L} comprising the amino acid sequence set forth in SEQ ID NO: 12. SEQ ID NO: 9, 12, and 52 are provided in Table 2. In certain embodiments, the scFv is designated as “P1-13”.

In certain embodiments, the anti-HPV scFv comprises a V_{H} Comprising the amino acid sequence set forth in SEQ ID NO: 9 and a V_{L} comprising the amino acid sequence set forth in SEQ ID NO: 12.

In certain embodiments, the anti-HPV scFv comprises a V_{H} that comprises a CDR1 comprising the amino acid sequence set forth in SEQ ID NO: 3 or a conservative modification thereof, a CDR2 comprising the amino acid sequence set forth in SEQ ID NO: 4 or a conservative modification thereof, and a CDR3 comprising the amino acid sequence set forth in SEQ ID NO: 5 or a conservative modification thereof. SEQ ID NOs: 3-5 are provided in Table 2.

In certain embodiments, the anti-HPV scFv comprises a V_{L} that comprises a CDR1 comprising the amino acid sequence set forth in SEQ ID NO: 6 or a conservative modification thereof, a CDR2 comprising the amino acid sequence set forth in SEQ ID NO: 7 or a conservative modification thereof, and a CDR3 comprising the amino acid sequence set forth in SEQ ID NO: 11 or a conservative modification thereof. SEQ ID NOs: 6, 7, and 11 are provided in Table 2.

In certain embodiments, the anti-HPV scFv comprises a V_{H} that comprises a CDR1 comprising the amino acid sequence set forth in SEQ ID NO: 3 or a conservative modification thereof, a CDR2 comprising the amino acid sequence set forth in SEQ ID NO: 4 or a conservative modification thereof, and a CDR3 comprising the amino acid sequence set forth in SEQ ID NO: 5 or a conservative modification thereof; and a V_{L} that comprises a CDR1 comprising the amino acid sequence set forth in SEQ ID NO: 6 or a conservative modification thereof, a CDR2 comprising the amino acid sequence set forth in SEQ ID NO: 7 or a conservative modification, and a CDR3 comprising the amino acid sequence set forth in SEQ ID NO: 11 or a conservative modification thereof.

In certain embodiments, the anti-HPV scFv comprises a V_{H} that comprises a CDR1 comprising the amino acid sequence set forth in SEQ ID NO: 3, a CDR2 comprising the amino acid sequence set forth in SEQ ID NO: 4, and a CDR3 comprising the amino acid sequence set forth in SEQ ID NO: 5; and a V_{L} that comprises a CDR1 comprising the amino acid sequence set forth in SEQ ID NO: 6, a CDR2 comprising the amino acid sequence set forth in SEQ ID NO: 7, and a CDR3 comprising the amino acid sequence set forth in SEQ ID NO: 11.

In certain embodiments, the anti-HPV scFv comprises a V_{H} Comprising the amino acid sequence set forth in SEQ ID NO: 9, and a V_{L} comprising the amino acid sequence set forth in SEQ ID NO: 12. In certain embodiments, the V_{H} and V_{L} are linked via a linker. In certain embodiments, the linker comprises or consists of the amino acid sequence set forth in SEQ ID NO: 44, SEQ ID NO: 45, SEQ ID NO: 46, SEQ ID NO: 47, SEQ ID NO: 48, or SEQ ID NO: 49. In certain embodiments, the variable regions are linked one after another such that a heavy chain variable region (V_{H}) is position at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 2

| CDRs | 1 | 2 | 3 |
|---|---|---|---|
| V_{H} | GFTFSDYY [SEQ ID | IYGGGGST [SEQ ID | AKDRWGYSSGWHNFDF |
| | NO: 3] | NO: 4] | [SEQ ID NO: 5] |
| V_{L} | QSVLRSGNNKND | WAS | QQSYNEPIT [SEQ ID |
| | [SEQ ID NO: 6] | [SEQ ID NO: 7] | NO: 11] |

| | |
|---|---|
| Full V_{H} | EVQLLESGGGLVQPGGSLRLSCAASGFTFSDYYMSWVRQAPGKGLEWVSGIYGGGGSTR |
| | YADSVKGRFTISRDNSKNTLYLQMNSLRAEDTAVYYCAKDRWGYSSGWHNFDFWGQGTL |
| | VTVSS [SEQ ID NO: 9] |
| Full V_{L} | DIVMTQSPDSLAVSLGERATINCKSSQSVLRSYNNKNDLAWYQQKPGQPPKLLI |
| | YWASTRESGVPDRFSGSGSGTDETLTISSLQAEDVAVYYCQQSYNEPITFGQGT |
| | KVEIK [SEQ ID NO: 12] |
| DNA for | GAAGTGCAGCTGCTGGAATCTGGCGGAGGACTGGTTCAACCTGGCGGCTCTCTG |
| Full V_{H} | AGACTGTCTTGTGCCGCCAGCGGCTTCACCTTCAGCGACTACTACATGAGCTGG |
| | GTCCGACAGGCCCCTGGCAAAGGACTTGAATGGGTGTCCGGCATCTATGGCGGC |
| | GGAGGCTCTACAAGATACGCCGACTCTGTGAAGGGCAGATTCACCATCAGCCGG |
| | GACAACAGCAAGAACACCCTGTACCTGCAGATGAACAGCCTGAGAGCCGAGGAC |
| | ACCGCCGTGTACTACTGCGCCAAAGACAGATGGGGCTACAGCAGCGGCTGGCAC |
| | AACTTCGATTTTTGGGGCCAGGGCACCCTGGTCACCGTTTCTTCT [SEQ ID |
| | NO: 52] |

In certain embodiments, the anti-HPV scFv is an scFv-Fc fusion protein or a full-length human IgG with V_{H} and V_{L} regions or CDRs selected from Table 3. In certain embodiments, the anti-HPV scFv comprises a V_{H} comprising the amino acid sequence set forth in SEQ ID NO: 9. An exemplary nucleotide sequence encoding the amino acid sequence of SEQ ID NO: 9 is set forth in SEQ ID NO: 52. In certain embodiments, the anti-HPV scFv comprises a V_{L} comprising the amino acid sequence set forth in SEQ ID NO: 15. SEQ ID NO: 9, 15, and 52 are provided in Table 3. In certain embodiments, the scFv is designated as “P1-75”.

In certain embodiments, the anti-HPV scFv comprises a V_{H} Comprising the amino acid sequence set forth in SEQ ID NO: 9 and a V_{L} comprising the amino acid sequence set forth in SEQ ID NO: 15.

In certain embodiments, the anti-HPV scFv comprises a V_{H} that comprises a CDR1 comprising the amino acid sequence set forth in SEQ ID NO: 3 or a conservative modification thereof, a CDR2 comprising the amino acid sequence set forth in SEQ ID NO: 4 or a conservative modification thereof, and a CDR3 comprising the amino acid sequence set forth in SEQ ID NO: 5 or a conservative modification thereof. SEQ ID NOs: 3-5 are provided in Table 3.

In certain embodiments, the anti-HPV scFv comprises a V_{L} that comprises a CDR1 comprising the amino acid sequence set forth in SEQ ID NO: 13 or a conservative modification thereof, a CDR2 comprising the amino acid sequence set forth in SEQ ID NO: 7 or a conservative modification thereof, and a CDR3 comprising the amino acid sequence set forth in SEQ ID NO: 14 or a conservative modification thereof. SEQ ID NOs: 7, 13, and 14 are provided in Table 3.

In certain embodiments, the anti-HPV scFv comprises a V_{H} that comprises a CDR1 comprising the amino acid sequence set forth in SEQ ID NO: 3 or a conservative modification thereof, a CDR2 comprising the amino acid sequence set forth in SEQ ID NO: 4 or a conservative modification thereof, and a CDR3 comprising the amino acid sequence set forth in SEQ ID NO: 5 or a conservative modification thereof; and a V_{L} that comprises a CDR1 comprising the amino acid sequence set forth in SEQ ID NO: 13 or a conservative modification thereof, a CDR2 comprising the amino acid sequence set forth in SEQ ID NO: 7 or a conservative modification, and a CDR3 comprising the amino acid sequence set forth in SEQ ID NO: 14 or a conservative modification thereof.

In certain embodiments, the anti-HPV scFv comprises a V_{H} that comprises a CDR1 comprising the amino acid sequence set forth in SEQ ID NO: 3, a CDR2 comprising the amino acid sequence set forth in SEQ ID NO: 4, and a CDR3 comprising the amino acid sequence set forth in SEQ ID NO: 5; and a V_{L} that comprises a CDR1 comprising the amino acid sequence set forth in SEQ ID NO: 13, a CDR2 comprising the amino acid sequence set forth in SEQ ID NO: 7, and a CDR3 comprising the amino acid sequence set forth in SEQ ID NO: 14.

In certain embodiments, the anti-HPV scFv comprises a V_{H} comprising the amino acid sequence set forth in SEQ ID NO: 9, and a V_{L} comprising the amino acid sequence set forth in SEQ ID NO: 15. In certain embodiments, the V_{H} and V_{L} are linked via a linker. In certain embodiments, the linker comprises or consists of the amino acid sequence set forth in SEQ ID NO: 44, SEQ ID NO: 45, SEQ ID NO: 46, SEQ ID NO: 47, SEQ ID NO: 48, or SEQ ID NO: 49. In certain embodiments, the variable regions are linked one after another such that a heavy chain variable region (V_{H}) is position at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 3

| CDRs | 1 | 2 | 3 |
|---|---|---|---|
| V_{H} | GFTFSDYY [SEQ ID | IYGGGGST [SEQ ID | AKDRWGYSSGWHNFDF |
| | NO: 3] | NO: 4] | [SEQ ID NO: 5] |
| V_{L} | QSVLYSYNNKND | WAS | QQSYEEPIT |
| | [SEQ ID NO: 13] | [SEQ ID NO: 7] | [SEQ ID NO: 14] |

| | |
|---|---|
| Full V_{H} | EVQLLESGGGLVQPGGSLRLSCAASGFTFSDYYMSWVRQAPGKGLEWVSGIYGGGGSTR |
| | YADSVKGRFTISRDNSKNTLYLQMNSLRAEDTAVYYCAKDRWGYSSGWHNFDFWGQGTL |
| | VTVSS [SEQ ID NO: 9] |
| Full V_{L} | DIVMTQSPDSLAVSLGERATINCKSSQSVLYSYNNKNDLAWYQQKPGQPPKLLIYWAST |
| | RESGVPDRFSGSGSGTDFTLTISSLQAEDVAVYYCQQSYEEPITFGQGTKVEIK [SEQ |
| | ID NO: 15] |
| DNA for | GAAGTGCAGCTGCTGGAATCTGGCGGAGGACTGGTTCAACCTGGCGGCTCTCTG |
| Full V_{H} | AGACTGTCTTGTGCCGCCAGCGGCTTCACCTTCAGCGACTACTACATGAGCTGG |
| | GTCCGACAGGCCCCTGGCAAAGGACTTGAATGGGTGTCCGGCATCTATGGCGGC |
| | GGAGGCTCTACAAGATACGCCGACTCTGTGAAGGGCAGATTCACCATCAGCCGG |
| | GACAACAGCAAGAACACCCTGTACCTGCAGATGAACAGCCTGAGAGCCGAGGAC |
| | ACCGCCGTGTACTACTGCGCCAAAGACAGATGGGGCTACAGCAGCGGCTGGCAC |
| | AACTTCGATTTTTGGGGCCAGGGCACCCTGGTCACCGTTTCTTCT [SEQ ID |
| | NO: 52] |

In certain embodiments, the anti-HPV scFv is an scFv-Fc fusion protein or a full-length human IgG with V_{H} and V_{L} regions or CDRs selected from Table 4. In certain embodiments, the anti-HPV scFv comprises a V_{H} comprising the amino acid sequence set forth in SEQ ID NO: 9. An exemplary nucleotide sequence encoding the amino acid sequence of SEQ ID NO: 9 is set forth in SEQ ID NO: 52. In certain embodiments, the anti-HPV scFv comprises a V_{L} comprising the amino acid sequence set forth in SEQ ID NO: 17. SEQ ID NO: 9, 17, and 52 are provided in Table 4. In certain embodiments, the scFv is designated as “P1-80”.

In certain embodiments, the anti-HPV scFv comprises a V_{H} Comprising the amino acid sequence set forth in SEQ ID NO: 9 and a V_{L} comprising the amino acid sequence set forth in SEQ ID NO: 17.

In certain embodiments, the anti-HPV scFv comprises a V_{H} that comprises a CDR1 comprising the amino acid sequence set forth in SEQ ID NO: 3 or a conservative modification thereof, a CDR2 comprising the amino acid sequence set forth in SEQ ID NO: 4 or a conservative modification thereof, and a CDR3 comprising the amino acid sequence set forth in SEQ ID NO: 5 or a conservative modification thereof. SEQ ID NOs: 3-5 are provided in Table 4.

In certain embodiments, the anti-HPV scFv comprises a V_{L} that comprises a CDR1 comprising the amino acid sequence set forth in SEQ ID NO: 6 or a conservative modification thereof, a CDR2 comprising the amino acid sequence set forth in SEQ ID NO: 7 or a conservative modification thereof, and a CDR3 comprising the amino acid sequence set forth in SEQ ID NO: 16 or a conservative modification thereof. SEQ ID NOs: 5, 6, and 16 are provided in Table 4.

In certain embodiments, the anti-HPV scFv comprises a V_{H} that comprises a CDR1 comprising the amino acid sequence set forth in SEQ ID NO: 3 or a conservative modification thereof, a CDR2 comprising the amino acid sequence set forth in SEQ ID NO: 4 or a conservative modification thereof, and a CDR3 comprising the amino acid sequence set forth in SEQ ID NO: 5 or a conservative modification thereof; and a V_{L} that comprises a CDR1 comprising the amino acid sequence set forth in SEQ ID NO: 6 or a conservative modification thereof, a CDR2 comprising the amino acid sequence set forth in SEQ ID NO: 7 or a conservative modification, and a CDR3 comprising the amino acid sequence set forth in SEQ ID NO: 16 or a conservative modification thereof.

In certain embodiments, the anti-HPV scFv comprises a V_{H} that comprises a CDR1 comprising the amino acid sequence set forth in SEQ ID NO: 3, a CDR2 comprising the amino acid sequence set forth in SEQ ID NO: 4, and a CDR3 comprising the amino acid sequence set forth in SEQ ID NO: 5; and a V_{L} that comprises a CDR1 comprising the amino acid sequence set forth in SEQ ID NO: 6, a CDR2 comprising the amino acid sequence set forth in SEQ ID NO: 7, and a CDR3 comprising the amino acid sequence set forth in SEQ ID NO: 16.

In certain embodiments, the anti-HPV scFv comprises a V_{H} Comprising the amino acid sequence set forth in SEQ ID NO: 9, and a V_{L} comprising the amino acid sequence set forth in SEQ ID NO: 17. In certain embodiments, the V_{H} and V_{L} are linked via a linker. In certain embodiments, the linker comprises or consists of the amino acid sequence set forth in SEQ ID NO: 44, SEQ ID NO: 45, SEQ ID NO: 46, SEQ ID NO: 47, SEQ ID NO: 48, or SEQ ID NO: 49. In certain embodiments, the variable regions are linked one after another such that a heavy chain variable region (V_{H}) is position at the N-terminus. In certain embodiments, the variable regions are positioned from the N- to the C-terminus: V_{H}—V_{L}. In certain embodiments, a light chain variable region (V_{L}) is positioned at the N-terminus. In certain embodiments, the variable regions are positioned from the N- to the C-terminus: V_{L}-V_{H}.

TABLE 4

| CDRs | 1 | 2 | 3 |
|---|---|---|---|
| V_{H} | GFTFSDYY [SEQ ID | IYGGGGST [SEQ ID | AKDRWGYSSGWHNFDF |
| | NO: 3] | NO: 4] | [SEQ ID NO: 5] |
| V_{L} | QSVLRSGNNKND | WAS | QQSYYSPIT |
| | [SEQ ID NO: 6] | [SEQ ID NO: 7] | [SEQ ID NO: 16] |

| | |
|---|---|
| Full V_{H} | EVQLLESGGGLVQPGGSLRLSCAASGFTFSDYYMSWVRQAPGKGLEWVSGIYGGGGSTR |
| | YADSVKGRFTISRDNSKNTLYLQMNSLRAEDTAVYYCAKDRWGYSSGWHNFDFWGQGTL |
| | VTVSS [SEQ ID NO: 9] |
| Full V_{L} | DIVMTQSPDSLAVSLGERATINCKSSQSVLRSYNNKNDLAWYQQKPGQPPKLLIYWAST |
| | RESGVPDRFSGSGSGTDFTLTISSLQAEDVAVYYCQQSYYSPITFGQGTKVEIK [SEQ |
| | ID NO: 17] |
| DNA for | GAAGTGCAGCTGCTGGAATCTGGCGGAGGACTGGTTCAACCTGGCGGCTCTCTG |
| Full V_{H} | AGACTGTCTTGTGCCGCCAGCGGCTTCACCTTCAGCGACTACTACATGAGCTGG |
| | GTCCGACAGGCCCCTGGCAAAGGACTTGAATGGGTGTCCGGCATCTATGGCGGC |
| | GGAGGCTCTACAAGATACGCCGACTCTGTGAAGGGCAGATTCACCATCAGCCGG |
| | GACAACAGCAAGAACACCCTGTACCTGCAGATGAACAGCCTGAGAGCCGAGGAC |
| | ACCGCCGTGTACTACTGCGCCAAAGACAGATGGGGCTACAGCAGCGGCTGGCAC |
| | AACTTCGATTTTTGGGGCCAGGGCACCCTGGTCACCGTTTCTTCT [SEQ ID |
| | NO: 10] |

In certain embodiments, the anti-HPV scFv is an scFv-Fc fusion protein or a full-length human IgG with V_{H} and V_{L} regions or CDRs selected from Table 5. In certain embodiments, the anti-HPV scFv comprises a V_{H} comprising the amino acid sequence set forth in SEQ ID NO: 9. An exemplary nucleotide sequence encoding the amino acid sequence of SEQ ID NO: 9 is set forth in SEQ ID NO: 52. In certain embodiments, the anti-HPV scFv comprises a V_{L} comprising the amino acid sequence set forth in SEQ ID NO: 20. SEQ ID NO: 9, 20, and 52 are provided in Table 5. In certain embodiments, the scFv is designated as “P2-75-1”.

In certain embodiments, the anti-HPV scFv comprises a V_{H} Comprising the amino acid sequence set forth in SEQ ID NO: 9 and a V_{L} comprising the amino acid sequence set forth in SEQ ID NO: 20.

In certain embodiments, the anti-HPV scFv comprises a V_{H} that comprises a CDR1 comprising the amino acid sequence set forth in SEQ ID NO: 3 or a conservative modification thereof, a CDR2 comprising the amino acid sequence set forth in SEQ ID NO: 4 or a conservative modification thereof, and a CDR3 comprising the amino acid sequence set forth in SEQ ID NO: 5 or a conservative modification thereof. SEQ ID NOs: 3-5 are provided in Table 5.

In certain embodiments, the anti-HPV scFv comprises a V_{L} that comprises a CDR1 comprising the amino acid sequence set forth in SEQ ID NO: 18 or a conservative modification thereof, a CDR2 comprising the amino acid sequence set forth in SEQ ID NO: 7 or a conservative modification thereof, and a CDR3 comprising the amino acid sequence set forth in SEQ ID NO: 19 or a conservative modification thereof. SEQ ID NOs: 7, 18, and 19 are provided in Table 5.

In certain embodiments, the anti-HPV scFv comprises a V_{H} that comprises a CDR1 comprising the amino acid sequence set forth in SEQ ID NO: 3 or a conservative modification thereof, a CDR2 comprising the amino acid sequence set forth in SEQ ID NO: 4 or a conservative modification thereof, and a CDR3 comprising the amino acid sequence set forth in SEQ ID NO: 5 or a conservative modification thereof; and a V_{L} that comprises a CDR1 comprising the amino acid sequence set forth in SEQ ID NO: 18 or a conservative modification thereof, a CDR2 comprising the amino acid sequence set forth in SEQ ID NO: 7 or a conservative modification, and a CDR3 comprising the amino acid sequence set forth in SEQ ID NO: 19 or a conservative modification thereof.

In certain embodiments, the anti-HPV scFv comprises a V_{H} that comprises a CDR1 comprising the amino acid sequence set forth in SEQ ID NO: 3, a CDR2 comprising the amino acid sequence set forth in SEQ ID NO: 4, and a CDR3 comprising the amino acid sequence set forth in SEQ ID NO: 5; and a V_{L} that comprises a CDR1 comprising the amino acid sequence set forth in SEQ ID NO: 18, a CDR2 comprising the amino acid sequence set forth in SEQ ID NO: 7, and a CDR3 comprising the amino acid sequence set forth in SEQ ID NO: 19.

In certain embodiments, the anti-HPV scFv comprises a V_{H} comprising the amino acid sequence set forth in SEQ ID NO: 9, and a V_{L} comprising the amino acid sequence set forth in SEQ ID NO: 20. In certain embodiments, the V_{H} and V_{L} are linked via a linker. In certain embodiments, the linker comprises or consists of the amino acid sequence set forth in SEQ ID NO: 44, SEQ ID NO: 45, SEQ ID NO: 46, SEQ ID NO: 47, SEQ ID NO: 48, or SEQ ID NO: 49. In certain embodiments, the variable regions are linked one after another such that a heavy chain variable region (V_{H}) is position at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 5

| CDRs | 1 | 2 | 3 |
|---|---|---|---|
| V_{H} | GFTFSDYY [SEQ ID | IYGGGGST [SEQ ID | AKDRWGYSSGWHNFDF |
| | NO: 3] | NO: 4] | [SEQ ID NO: 5] |
| V_{L} | QSVLRSGNNKNR | WAS | QQYTRRPIT |
| | [SEQ ID NO: 18] | [SEQ ID NO: 7] | [SEQ ID NO: 19] |

| | |
|---|---|
| Full V_{H} | EVQLLESGGGLVQPGGSLRLSCAASGFTFSDYYMSWVRQAPGKGLEWVSGIYGGGGSTR |
| | YADSVKGRFTISRDNSKNTLYLQMNSLRAEDTAVYYCAKDRWGYSSGWHNFDFWGQGTL |
| | VTVSS [SEQ ID NO: 9] |
| Full V_{L} | DIVMTQSPDSLAVSLGERATINCKSSQSVLRSGNNKNRLAWYQQKPGQPPKLLIYWAST |
| | RESGVPDRFSGSGSGTDFTLTISSLQAEDVAVYYCQQYTRRPITFGQGTKVEIK [SEQ |
| | ID NO: 20] |
| DNA for | GAAGTGCAGCTGCTGGAATCTGGCGGAGGACTGGTTCAACCTGGCGGCTCTCTG |
| Full V_{H} | AGACTGTCTTGTGCCGCCAGCGGCTTCACCTTCAGCGACTACTACATGAGCTGG |
| | GTCCGACAGGCCCCTGGCAAAGGACTTGAATGGGTGTCCGGCATCTATGGCGGC |
| | GGAGGCTCTACAAGATACGCCGACTCTGTGAAGGGCAGATTCACCATCAGCCGG |
| | GACAACAGCAAGAACACCCTGTACCTGCAGATGAACAGCCTGAGAGCCGAGGAC |
| | ACCGCCGTGTACTACTGCGCCAAAGACAGATGGGGCTACAGCAGCGGCTGGCAC |
| | AACTTCGATTTTTGGGGCCAGGGCACCCTGGTCACCGTTTCTTCT [SEQ ID |
| | NO: 52] |

In certain embodiments, the anti-HPV scFv is an scFv-Fc fusion protein or a full-length human IgG with V_{H} and V_{L} regions or CDRs selected from Table 6. In certain embodiments, the anti-HPV scFv comprises a V_{H} comprising the amino acid sequence set forth in SEQ ID NO: 9. An exemplary nucleotide sequence encoding the amino acid sequence of SEQ ID NO: 9 is set forth in SEQ ID NO: 52. In certain embodiments, the anti-HPV scFv comprises a V_{L} comprising the amino acid sequence set forth in SEQ ID NO: 22. SEQ ID NO: 9, 22, and 52 are provided in Table 6. In certain embodiments, the scFv is designated as “P2-75-5”.

In certain embodiments, the anti-HPV scFv comprises a V_{H} Comprising the amino acid sequence set forth in SEQ ID NO: 9 and a V_{L} comprising the amino acid sequence set forth in SEQ ID NO: 22.

In certain embodiments, the anti-HPV scFv comprises a V_{H} that comprises a CDR1 comprising the amino acid sequence set forth in SEQ ID NO: 3 or a conservative modification thereof, a CDR2 comprising the amino acid sequence set forth in SEQ ID NO: 4 or a conservative modification thereof, and a CDR3 comprising the amino acid sequence set forth in SEQ ID NO: 5 or a conservative modification thereof. SEQ ID NOs: 3-5 are provided in Table 6.

In certain embodiments, the anti-HPV scFv comprises a V_{L} that comprises a CDR1 comprising the amino acid sequence set forth in SEQ ID NO: 6 or a conservative modification thereof, a CDR2 comprising the amino acid sequence set forth in SEQ ID NO: 7 or a conservative modification thereof, and a CDR3 comprising the amino acid sequence set forth in SEQ ID NO: 21 or a conservative modification thereof. SEQ ID NOs: 6, 7, and 21 are provided in Table 6.

In certain embodiments, the anti-HPV scFv comprises a V_{H} that comprises a CDR1 comprising the amino acid sequence set forth in SEQ ID NO: 3 or a conservative modification thereof, a CDR2 comprising the amino acid sequence set forth in SEQ ID NO: 4 or a conservative modification thereof, and a CDR3 comprising the amino acid sequence set forth in SEQ ID NO: 5 or a conservative modification thereof; and a V_{L} that comprises a CDR1 comprising the amino acid sequence set forth in SEQ ID NO: 6 or a conservative modification thereof, a CDR2 comprising the amino acid sequence set forth in SEQ ID NO: 7 or a conservative modification, and a CDR3 comprising the amino acid sequence set forth in SEQ ID NO: 21 or a conservative modification thereof.

In certain embodiments, the anti-HPV scFv comprises a V_{H} that comprises a CDR1 comprising the amino acid sequence set forth in SEQ ID NO: 3, a CDR2 comprising the amino acid sequence set forth in SEQ ID NO: 4, and a CDR3 comprising the amino acid sequence set forth in SEQ ID NO: 5; and a V_{L} that comprises a CDR1 comprising the amino acid sequence set forth in SEQ ID NO: 6, a CDR2 comprising the amino acid sequence set forth in SEQ ID NO: 7, and a CDR3 comprising the amino acid sequence set forth in SEQ ID NO: 21.

In certain embodiments, the anti-HPV scFv comprises a V_{H} comprising the amino acid sequence set forth in SEQ ID NO: 9, and a V_{L} comprising the amino acid sequence set forth in SEQ ID NO: 22. In certain embodiments, the V_{H} and V_{L} are linked via a linker. In certain embodiments, the linker comprises or consists of the amino acid sequence set forth in SEQ ID NO: 44, SEQ ID NO: 45, SEQ ID NO: 46, SEQ ID NO: 47, SEQ ID NO: 48, or SEQ ID NO: 49. In certain embodiments, the variable regions are linked one after another such that a heavy chain variable region (V_{H}) is position at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 6

| CDRs | 1 | 2 | 3 |
|---|---|---|---|
| V_{H} | GFTFSDYY [SEQ ID | IYGGGGST [SEQ ID | AKDRWGYSSGWHNFDF |
| | NO: 3] | NO: 4] | [SEQ ID NO: 5] |
| V_{L} | QSVLRSGNNKND | WAS | QQSYSEPLT |
| | [SEQ ID NO: 6] | [SEQ ID NO: 7] | [SEQ ID NO: 21] |

| | |
|---|---|
| Full V_{H} | EVQLLESGGGLVQPGGSLRLSCAASGFTFSDYYMSWVRQAPGKGLEWVSGIYGGGGSTR |
| | YADSVKGRFTISRDNSKNTLYLQMNSLRAEDTAVYYCAKDRWGYSSGWHNFDFWGQGTL |
| | VTVSS [SEQ ID NO: 9] |
| Full V_{L} | DIVMTQSPDSLAVSLGERATINCKSSQSVLRSGNNKNDLAWYQQKPGQPPKLLIYWAST |
| | RESGVPDRFSGSGSGTDFTLTISSLQAEDVAVYYCQQSYSEPLTFGQGTKVEIK |
| | [SEQ ID NO: 22] |
| DNA for | GAAGTGCAGCTGCTGGAATCTGGCGGAGGACTGGTTCAACCTGGCGGCTCTCTG |
| Full V_{H} | AGACTGTCTTGTGCCGCCAGCGGCTTCACCTTCAGCGACTACTACATGAGCTGG |
| | GTCCGACAGGCCCCTGGCAAAGGACTTGAATGGGTGTCCGGCATCTATGGCGGC |
| | GGAGGCTCTACAAGATACGCCGACTCTGTGAAGGGCAGATTCACCATCAGCCGG |
| | GACAACAGCAAGAACACCCTGTACCTGCAGATGAACAGCCTGAGAGCCGAGGAC |
| | ACCGCCGTGTACTACTGCGCCAAAGACAGATGGGGCTACAGCAGCGGCTGGCAC |
| | AACTTCGATTTTTGGGGCCAGGGCACCCTGGTCACCGTTTCTTCT [SEQ ID |
| | NO: 52] |

In certain embodiments, the anti-HPV scFv is an scFv-Fc fusion protein or a full-length human IgG with V_{H} and V_{L} regions or CDRs selected from Table 7. In certain embodiments, the anti-HPV scFv comprises a V_{H} comprising the amino acid sequence set forth in SEQ ID NO: 9. An exemplary nucleotide sequence encoding the amino acid sequence of SEQ ID NO: 9 is set forth in SEQ ID NO: 52. In certain embodiments, the anti-HPV scFv comprises a V_{L} comprising the amino acid sequence set forth in SEQ ID NO: 24. SEQ ID NO: 9, 24, and 52 are provided in Table 7. In certain embodiments, the scFv is designated as “P3-30”.

In certain embodiments, the anti-HPV scFv comprises a V_{H} Comprising the amino acid sequence set forth in SEQ ID NO: 9 and a V_{L} comprising the amino acid sequence set forth in SEQ ID NO: 24.

In certain embodiments, the anti-HPV scFv comprises a V_{H} that comprises a CDR1 comprising the amino acid sequence set forth in SEQ ID NO: 3 or a conservative modification thereof, a CDR2 comprising the amino acid sequence set forth in SEQ ID NO: 4 or a conservative modification thereof, and a CDR3 comprising the amino acid sequence set forth in SEQ ID NO: 5 or a conservative modification thereof. SEQ ID NOs: 3-5 are provided in Table 7.

In certain embodiments, the anti-HPV scFv comprises a V_{L} that comprises a CDR1 comprising the amino acid sequence set forth in SEQ ID NO: 6 or a conservative modification thereof, a CDR2 comprising the amino acid sequence set forth in SEQ ID NO: 7 or a conservative modification thereof, and a CDR3 comprising the amino acid sequence set forth in SEQ ID NO: 23 or a conservative modification thereof. SEQ ID NOs: 6, 7, and 23 are provided in Table 7.

In certain embodiments, the anti-HPV scFv comprises a V_{H} that comprises a CDR1 comprising the amino acid sequence set forth in SEQ ID NO: 3 or a conservative modification thereof, a CDR2 comprising the amino acid sequence set forth in SEQ ID NO: 4 or a conservative modification thereof, and a CDR3 comprising the amino acid sequence set forth in SEQ ID NO: 5 or a conservative modification thereof; and a V_{L} that comprises a CDR1 comprising the amino acid sequence set forth in SEQ ID NO: 6 or a conservative modification thereof, a CDR2 comprising the amino acid sequence set forth in SEQ ID NO: 7 or a conservative modification, and a CDR3 comprising the amino acid sequence set forth in SEQ ID NO: 23 or a conservative modification thereof.

In certain embodiments, the anti-HPV scFv comprises a V_{H} that comprises a CDR1 comprising the amino acid sequence set forth in SEQ ID NO: 3, a CDR2 comprising the amino acid sequence set forth in SEQ ID NO: 4, and a CDR3 comprising the amino acid sequence set forth in SEQ ID NO: 5; and a V_{L} that comprises a CDR1 comprising the amino acid sequence set forth in SEQ ID NO: 6, a CDR2 comprising the amino acid sequence set forth in SEQ ID NO: 7, and a CDR3 comprising the amino acid sequence set forth in SEQ ID NO: 23.

In certain embodiments, the anti-HPV scFv comprises a V_{H} comprising the amino acid sequence set forth in SEQ ID NO: 9, and a V_{L} comprising the amino acid sequence set forth in SEQ ID NO: 24. In certain embodiments, the V_{H} and V_{L} are linked via a linker. In certain embodiments, the linker comprises or consists of the amino acid sequence set forth in SEQ ID NO: 44, SEQ ID NO: 45, SEQ ID NO: 46, SEQ ID NO: 47, SEQ ID NO: 48, or SEQ ID NO: 49. In certain embodiments, the variable regions are linked one after another such that a heavy chain variable region (V_{H}) is position at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 7

| CDRs | 1 | 2 | 3 |
|---|---|---|---|
| V_{H} | GFTFSDYY [SEQ ID | IYGGGGST [SEQ ID | AKDRWGYSSGWHNFDF |
| | NO: 3] | NO: 4] | [SEQ ID NO: 5] |
| V_{L} | QSVLRSGNNKND | WAS | QQSYNAPLT [SEQ ID |
| | [SEQ ID NO: 6] | [SEQ ID NO: 7] | NO: 23] |

| | |
|---|---|
| Full V_{H} | EVQLLESGGGLVQPGGSLRLSCAASGFTFSDYYMSWVRQAPGKGLEWVSGIYGGGGSTR |
| | YADSVKGRFTISRDNSKNTLYLQMNSLRAEDTAVYYCAKDRWGYSSGWHNFDFWGQGTL |
| | VTVSS [SEQ ID NO: 9] |
| Full V_{L} | DIVMTQSPDSLAVSLGERATINCKSSQSVLRSYNNKNDLAWYQQKPGQPPKLLI |
| | YWASTRESGVPDRFSGSGSGTDETLTISSLQAEDVAVYYCQQSYNAPLTFGQGT |
| | KVEIK [SEQ ID NO: 24] |
| DNA for | GAAGTGCAGCTGCTGGAATCTGGCGGAGGACTGGTTCAACCTGGCGGCTCTCTG |
| Full V_{H} | AGACTGTCTTGTGCCGCCAGCGGCTTCACCTTCAGCGACTACTACATGAGCTGG |
| | GTCCGACAGGCCCCTGGCAAAGGACTTGAATGGGTGTCCGGCATCTATGGCGGC |
| | GGAGGCTCTACAAGATACGCCGACTCTGTGAAGGGCAGATTCACCATCAGCCGG |
| | GACAACAGCAAGAACACCCTGTACCTGCAGATGAACAGCCTGAGAGCCGAGGAC |
| | ACCGCCGTGTACTACTGCGCCAAAGACAGATGGGGCTACAGCAGCGGCTGGCAC |
| | AACTTCGATTTTTGGGGCCAGGGCACCCTGGTCACCGTTTCTTCT [SEQ ID |
| | NO: 52] |

In certain embodiments, the anti-HPV scFv is an scFv-Fc fusion protein or a full-length human IgG with V_{H} and V_{L} regions or CDRs selected from Table 8. In certain embodiments, the anti-HPV scFv comprises a V_{H} comprising the amino acid sequence set forth in SEQ ID NO: 9. An exemplary nucleotide sequence encoding the amino acid sequence of SEQ ID NO: 9 is set forth in SEQ ID NO: 52. In certain embodiments, the anti-HPV scFv comprises a V_{L} comprising the amino acid sequence set forth in SEQ ID NO: 26. SEQ ID NO: 9, 26, and 52 are provided in Table 8. In certain embodiments, the scFv is designated as “P4-10”.

In certain embodiments, the anti-HPV scFv comprises a V_{H} Comprising the amino acid sequence set forth in SEQ ID NO: 9 and a V_{L} comprising the amino acid sequence set forth in SEQ ID NO: 26.

In certain embodiments, the anti-HPV scFv comprises a V_{H} that comprises a CDR1 comprising the amino acid sequence set forth in SEQ ID NO: 3 or a conservative modification thereof, a CDR2 comprising the amino acid sequence set forth in SEQ ID NO: 4 or a conservative modification thereof, and a CDR3 comprising the amino acid sequence set forth in SEQ ID NO: 5 or a conservative modification thereof. SEQ ID NOs: 3-5 are provided in Table 8.

In certain embodiments, the anti-HPV scFv comprises a V_{L} that comprises a CDR1 comprising the amino acid sequence set forth in SEQ ID NO: 6 or a conservative modification thereof, a CDR2 comprising the amino acid sequence set forth in SEQ ID NO: 7 or a conservative modification thereof, and a CDR3 comprising the amino acid sequence set forth in SEQ ID NO: 25 or a conservative modification thereof. SEQ ID NOs: 6, 7, and 25 are provided in Table 8.

In certain embodiments, the anti-HPV scFv comprises a V_{H} that comprises a CDR1 comprising the amino acid sequence set forth in SEQ ID NO: 3 or a conservative modification thereof, a CDR2 comprising the amino acid sequence set forth in SEQ ID NO: 4 or a conservative modification thereof, and a CDR3 comprising the amino acid sequence set forth in SEQ ID NO: 5 or a conservative modification thereof; and a V_{L} that comprises a CDR1 comprising the amino acid sequence set forth in SEQ ID NO: 6 or a conservative modification thereof, a CDR2 comprising the amino acid sequence set forth in SEQ ID NO: 7 or a conservative modification, and a CDR3 comprising the amino acid sequence set forth in SEQ ID NO: 25 or a conservative modification thereof.

In certain embodiments, the anti-HPV scFv comprises a V_{H} that comprises a CDR1 comprising the amino acid sequence set forth in SEQ ID NO: 3, a CDR2 comprising the amino acid sequence set forth in SEQ ID NO: 4, and a CDR3 comprising the amino acid sequence set forth in SEQ ID NO: 5; and a V_{L} that comprises a CDR1 comprising the amino acid sequence set forth in SEQ ID NO: 6, a CDR2 comprising the amino acid sequence set forth in SEQ ID NO: 7, and a CDR3 comprising the amino acid sequence set forth in SEQ ID NO: 25.

In certain embodiments, the anti-HPV scFv comprises a V_{H} Comprising the amino acid sequence set forth in SEQ ID NO: 9, and a V_{L} comprising the amino acid sequence set forth in SEQ ID NO: 26. In certain embodiments, the V_{H} and V_{L} are linked via a linker. In certain embodiments, the linker comprises or consists of the amino acid sequence set forth in SEQ ID NO: 44, SEQ ID NO: 45, SEQ ID NO: 46, SEQ ID NO: 47, SEQ ID NO: 48, or SEQ ID NO: 49. In certain embodiments, the variable regions are linked one after another such that a heavy chain variable region (V_{H}) is position at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 8

| CDRs | 1 | 2 | 3 |
|---|---|---|---|
| V_{H} | GFTFSDYY [SEQ ID | IYGGGGST [SEQ ID | AKDRWGYSSGWHNFDF |
| | NO: 3] | NO: 4] | [SEQ ID NO: 5] |
| V_{L} | QSVLRSGNNKND | WAS | QQSYNSPIT [SEQ ID |
| | [SEQ ID NO: 6] | [SEQ ID NO: 7] | NO: 25] |

| | |
|---|---|
| Full V_{H} | EVQLLESGGGLVQPGGSLRLSCAASGFTFSDYYMSWVRQAPGKGLEWVSGIYGGGGSTR |
| | YADSVKGRFTISRDNSKNTLYLQMNSLRAEDTAVYYCAKDRWGYSSGWHNFDFWGQGTL |
| | VTVSS [SEQ ID NO: 9] |
| Full V_{L} | DIVMTQSPDSLAVSLGERATINCKSSQSVLRSYNNKNDLAWYQQKPGQPPKLLIYWAST |
| | RESGVPDRFSGSGSGTDFTLTISSLQAEDVAVYYCQQSYNSPITFGQGTKVEIK |
| | [SEQ ID NO: 26] |
| DNA for | GAAGTGCAGCTGCTGGAATCTGGCGGAGGACTGGTTCAACCTGGCGGCTCTCTG |
| Full V_{H} | AGACTGTCTTGTGCCGCCAGCGGCTTCACCTTCAGCGACTACTACATGAGCTGG |
| | GTCCGACAGGCCCCTGGCAAAGGACTTGAATGGGTGTCCGGCATCTATGGCGGC |
| | GGAGGCTCTACAAGATACGCCGACTCTGTGAAGGGCAGATTCACCATCAGCCGG |
| | GACAACAGCAAGAACACCCTGTACCTGCAGATGAACAGCCTGAGAGCCGAGGAC |
| | ACCGCCGTGTACTACTGCGCCAAAGACAGATGGGGCTACAGCAGCGGCTGGCAC |
| | AACTTCGATTTTTGGGGCCAGGGCACCCTGGTCACCGTTTCTTCT [SEQ ID |
| | NO: 52] |

In certain embodiments, the anti-HPV scFv is an scFv-Fc fusion protein or a full-length human IgG with V_{H} and V_{L} regions or CDRs selected from Table 9. In certain embodiments, the anti-HPV scFv comprises a V_{H} comprising the amino acid sequence set forth in SEQ ID NO: 9. An exemplary nucleotide sequence encoding the amino acid sequence of SEQ ID NO: 9 is set forth in SEQ ID NO: 52. In certain embodiments, the anti-HPV scFv comprises a V_{L} comprising the amino acid sequence set forth in SEQ ID NO: 28. SEQ ID NO: 9, 28, and 52 are provided in Table 9. In certain embodiments, the scFv is designated as “R4-D1”.

In certain embodiments, the anti-HPV scFv comprises a V_{H} Comprising the amino acid sequence set forth in SEQ ID NO: 9 and a V_{L} comprising the amino acid sequence set forth in SEQ ID NO: 28.

In certain embodiments, the anti-HPV scFv comprises a V_{H} that comprises a CDR1 comprising the amino acid sequence set forth in SEQ ID NO: 3 or a conservative modification thereof, a CDR2 comprising the amino acid sequence set forth in SEQ ID NO: 4 or a conservative modification thereof, and a CDR3 comprising the amino acid sequence set forth in SEQ ID NO: 5 or a conservative modification thereof. SEQ ID NOs: 3-5 are provided in Table 9.

In certain embodiments, the anti-HPV scFv comprises a V_{L} that comprises a CDR1 comprising the amino acid sequence set forth in SEQ ID NO: 6 or a conservative modification thereof, a CDR2 comprising the amino acid sequence set forth in SEQ ID NO: 7 or a conservative modification thereof, and a CDR3 comprising the amino acid sequence set forth in SEQ ID NO: 27 or a conservative modification thereof. SEQ ID NOs: 6, 7, and 27 are provided in Table 9.

In certain embodiments, the anti-HPV scFv comprises a V_{H} that comprises a CDR1 comprising the amino acid sequence set forth in SEQ ID NO: 3 or a conservative modification thereof, a CDR2 comprising the amino acid sequence set forth in SEQ ID NO: 4 or a conservative modification thereof, and a CDR3 comprising the amino acid sequence set forth in SEQ ID NO: 5 or a conservative modification thereof; and a V_{L} that comprises a CDR1 comprising the amino acid sequence set forth in SEQ ID NO: 6 or a conservative modification thereof, a CDR2 comprising the amino acid sequence set forth in SEQ ID NO: 7 or a conservative modification, and a CDR3 comprising the amino acid sequence set forth in SEQ ID NO: 27 or a conservative modification thereof.

In certain embodiments, the anti-HPV scFv comprises a V_{H} that comprises a CDR1 comprising the amino acid sequence set forth in SEQ ID NO: 3, a CDR2 comprising the amino acid sequence set forth in SEQ ID NO: 4, and a CDR3 comprising the amino acid sequence set forth in SEQ ID NO: 5; and a V_{L} that comprises a CDR1 comprising the amino acid sequence set forth in SEQ ID NO: 6, a CDR2 comprising the amino acid sequence set forth in SEQ ID NO: 7, and a CDR3 comprising the amino acid sequence set forth in SEQ ID NO: 27.

In certain embodiments, the anti-HPV scFv comprises a V_{H} Comprising the amino acid sequence set forth in SEQ ID NO: 9, and a V_{L} comprising the amino acid sequence set forth in SEQ ID NO: 28. In certain embodiments, the V_{H} and V_{L} are linked via a linker. In certain embodiments, the linker comprises or consists of the amino acid sequence set forth in SEQ ID NO: 44, SEQ ID NO: 45, SEQ ID NO: 46, SEQ ID NO: 47, SEQ ID NO: 48, or SEQ ID NO: 49. In certain embodiments, the variable regions are linked one after another such that a heavy chain variable region (V_{H}) is position at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 9

| CDRs | 1 | 2 | 3 |
|---|---|---|---|
| V_{H} | GFTFSDYY [SEQ ID | IYGGGGST [SEQ ID | AKDRWGYSSGWHNFDF |
| | NO: 3] | NO: 4] | [SEQ ID NO: 5] |
| V_{L} | QSVLRSGNNKND | WAS | QQSYSSPIT [SEQ ID |
| | [SEQ ID NO: 6] | [SEQ ID NO: 7] | NO: 27] |

| | |
|---|---|
| Full V_{H} | EVQLLESGGGLVQPGGSLRLSCAASGFTFSDYYMSWVRQAPGKGLEWVSGIYGGGGSTR |
| | YADSVKGRFTISRDNSKNTLYLQMNSLRAEDTAVYYCAKDRWGYSSGWHNFDFWGQGTL |
| | VTVSS [SEQ ID NO: 9] |
| Full V_{L} | DIVMTQSPDSLAVSLGERATINCKSSQSVLRSYNNKNDLAWYQQKPGQPPKLLI |
| | YWASTRESGVPDRFSGSGSGTDFTLTISSLQAEDVAVYYCQQSYSSPITFGQGT |
| | KVEIK [SEQ ID NO: 28] |
| DNA for | GAAGTGCAGCTGCTGGAATCTGGCGGAGGACTGGTTCAACCTGGCGGCTCTCTG |
| Full V_{H} | AGACTGTCTTGTGCCGCCAGCGGCTTCACCTTCAGCGACTACTACATGAGCTGG |
| | GTCCGACAGGCCCCTGGCAAAGGACTTGAATGGGTGTCCGGCATCTATGGCGGC |
| | GGAGGCTCTACAAGATACGCCGACTCTGTGAAGGGCAGATTCACCATCAGCCGG |
| | GACAACAGCAAGAACACCCTGTACCTGCAGATGAACAGCCTGAGAGCCGAGGAC |
| | ACCGCCGTGTACTACTGCGCCAAAGACAGATGGGGCTACAGCAGCGGCTGGCAC |
| | AACTTCGATTTTTGGGGCCAGGGCACCCTGGTCACCGTTTCTTCT [SEQ ID |
| | NO: 52] |

In certain embodiments, the anti-HPV scFv is an scFv-Fc fusion protein or a full-length human IgG with V_{H} and V_{L} regions or CDRs selected from Table 10. In certain embodiments, the anti-HPV scFv comprises a V_{H} comprising the amino acid sequence set forth in SEQ ID NO: 34. An exemplary nucleotide sequence encoding the amino acid sequence of SEQ ID NO: 34 is set forth in SEQ ID NO: 54. In certain embodiments, the anti-HPV scFv comprises a V_{L} comprising the amino acid sequence set forth in SEQ ID NO: 35. An exemplary nucleotide sequence encoding the amino acid sequence of SEQ ID NO: 35 is set forth in SEQ ID NO: 55. SEQ ID NO: 34, 35, 54, and 55 are provided in Table 10. In certain embodiments, the scFv is designated as “2A5”.

In certain embodiments, the anti-HPV scFv comprises a V_{H} Comprising the amino acid sequence set forth in SEQ ID NO: 34 and a V_{L} comprising the amino acid sequence set forth in SEQ ID NO: 35.

In certain embodiments, the anti-HPV scFv comprises a V_{H} that comprises a CDR1 comprising the amino acid sequence set forth in SEQ ID NO: 3 or a conservative modification thereof, a CDR2 comprising the amino acid sequence set forth in SEQ ID NO: 29 or a conservative modification thereof, and a CDR3 comprising the amino acid sequence set forth in SEQ ID NO: 30 or a conservative modification thereof. SEQ ID NOs: 3, 29, and 30 are provided in Table 10.

In certain embodiments, the anti-HPV scFv comprises a V_{L} that comprises a CDR1 comprising the amino acid sequence set forth in SEQ ID NO: 31 or a conservative modification thereof, a CDR2 comprising the amino acid sequence set forth in SEQ ID NO: 32 or a conservative modification thereof, and a CDR3 comprising the amino acid sequence set forth in SEQ ID NO: 33 or a conservative modification thereof. SEQ ID NOs: 31-33 are provided in Table 10.

In certain embodiments, the anti-HPV scFv comprises a V_{H} that comprises a CDR1 comprising the amino acid sequence set forth in SEQ ID NO: 3 or a conservative modification thereof, a CDR2 comprising the amino acid sequence set forth in SEQ ID NO: 29 or a conservative modification thereof, and a CDR3 comprising the amino acid sequence set forth in SEQ ID NO: 30 or a conservative modification thereof; and a V_{L} that comprises a CDR1 comprising the amino acid sequence set forth in SEQ ID NO: 31 or a conservative modification thereof, a CDR2 comprising the amino acid sequence set forth in SEQ ID NO: 32 or a conservative modification, and a CDR3 comprising the amino acid sequence set forth in SEQ ID NO: 33 or a conservative modification thereof.

In certain embodiments, the anti-HPV scFv comprises a V_{H} that comprises a CDR1 comprising the amino acid sequence set forth in SEQ ID NO: 3, a CDR2 comprising the amino acid sequence set forth in SEQ ID NO: 29, and a CDR3 comprising the amino acid sequence set forth in SEQ ID NO: 30; and a V_{L} that comprises a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33.

In certain embodiments, the anti-HPV scFv comprises a V_{H} comprising the amino acid sequence set forth in SEQ ID NO: 34, and a V_{L} comprising the amino acid sequence set forth in SEQ ID NO: 35. In certain embodiments, the V_{H} and V_{L} are linked via a linker. In certain embodiments, the linker comprises or consists of the amino acid sequence set forth in SEQ ID NO: 44, SEQ ID NO: 45, SEQ ID NO: 46, SEQ ID NO: 47, SEQ ID NO: 48, or SEQ ID NO: 49. In certain embodiments, the variable regions are linked one after another such that a heavy chain variable region (V_{H}) is position at the N-terminus. In certain embodiments, the variable regions are positioned from the N- to the C-terminus: V_{H}—V_{L}. In certain embodiments, a light chain variable region (V_{L}) is positioned at the N-terminus. In certain embodiments, the variable regions are positioned from the N- to the C-terminus: V_{L}-V_{H}.

TABLE 10

| CDRs | 1 | 2 | 3 |
|---|---|---|---|
| V_{H} | GFTFSDYY [SEQ ID | ISSSGSTI [SEQ ID | ARGPLTADAFDI [SEQ |
| | NO: 3] | NO: 29] | ID NO: 30] |
| V_{L} | SNIGSNT [SEQ ID NO: | NNN [SEQ ID NO: | ATWDDTRNGPE [SEQ |
| | 31] | 32] | ID NO: 33] |

| | |
|---|---|
| Full V_{H} | EVQLVQSGGGLVKPGGSLRLSCAASGFTFSDYYMSWIRQAPGKGLEWVSYISSSGSTIY |
| | YADSVKGRFTISRDNAKNSLYLQMNSLRAEDTAVYYCARGPLTADAFDIWGQGTMVTVS |
| | S [SEQ ID NO: 34] |
| Full V_{L} | QSVLTQPPSASGTPGQRVTISCSGSSSNIGSNTVNWYQQLPGTAPKLLIYNNNQ |
| | RPSGVPDRFSGSKSGTSAALAISGLQSEDEADYHCATWDDTRNGPEFGGGTKLT |
| | VL [SEQ ID NO: 35] |
| DNA for | GAAGTGCAGCTGGTTCAGTCTGGCGGCGGACTTGTGAAACCTGGCGGAAGCCTG |
| Full V_{H} | AGACTGAGCTGTGCCGCTTCCGGCTTCACCTTCAGCGACTACTACATGAGCTGG |
| | ATCAGACAGGCCCCTGGCAAAGGCCTGGAATGGGTGTCCTACATCAGCAGCAGC |
| | GGCAGCACCATCTACTACGCCGATAGCGTGAAGGGCAGATTCACCATCAGCCGG |
| | GACAACGCCAAGAACAGCCTGTACCTGCAGATGAACTCCCTGAGAGCCGAGGAC |
| | ACCGCCGTGTACTATTGTGCCAGAGGACCTCTGACCGCCGACGCCTTTGATATC |
| | TGGGGCCAGGGCACAATGGTCACCGTGTCATCT [SEQ ID NO: 54] |
| DNA for | CAGTCTGTGCTGACACAGCCTCCATCTGCCTCTGGCACACCTGGCCAGAGAGTG |
| Full V_{L} | ACCATCAGCTGTAGCGGCAGCAGCAGCAACATCGGCAGCAACACCGTGAACTGG |
| | TATCAGCAGCTGCCTGGCACAGCCCCTAAACTGCTGATCTACAACAACAACCAG |
| | CGGCCTAGCGGCGTGCCCGATAGATTTTCTGGCAGCAAGAGCGGCACAAGCGCC |
| | GCTCTGGCTATTTCTGGACTGCAGAGCGAGGACGAGGCCGATTACCATTGTGCC |
| | ACCTGGGACGACACCAGAAACGGCCCTGAATTTGGCGGAGGCACCAAGCTGACA |
| | GTGCTG [SEQ ID NO: 55] |

In certain embodiments, the anti-HPV scFv is an scFv-Fc fusion protein or a full-length human IgG with V_{H} and V_{L} regions or CDRs selected from Table 11. In certain embodiments, the anti-HPV scFv comprises a V_{H} comprising the amino acid sequence set forth in SEQ ID NO: 42. An exemplary nucleotide sequence encoding the amino acid sequence of SEQ ID NO: 42 is set forth in SEQ ID NO: 56. In certain embodiments, the anti-HPV scFv comprises a V_{L} comprising the amino acid sequence set forth in SEQ ID NO: 43. An exemplary nucleotide sequence encoding the amino acid sequence of SEQ ID NO: 43 is set forth in SEQ ID NO: 57. SEQ ID NO: 42, 43, 56, and 57 are provided in Table 11. In certain embodiments, the scFv is designated as “1B1”.

In certain embodiments, the anti-HPV scFv comprises a V_{H} Comprising the amino acid sequence set forth in SEQ ID NO: 42 and a V_{L} comprising the amino acid sequence set forth in SEQ ID NO: 43.

In certain embodiments, the anti-HPV scFv comprises a V_{H} that comprises a CDR1 comprising the amino acid sequence set forth in SEQ ID NO: 36 or a conservative modification thereof, a CDR2 comprising the amino acid sequence set forth in SEQ ID NO: 37 or a conservative modification thereof, and a CDR3 comprising the amino acid sequence set forth in SEQ ID NO: 38 or a conservative modification thereof. SEQ ID NOs: 37-38 are provided in Table 11.

In certain embodiments, the anti-HPV scFv comprises a V_{L} that comprises a CDR1 comprising the amino acid sequence set forth in SEQ ID NO: 39 or a conservative modification thereof, a CDR2 comprising the amino acid sequence set forth in SEQ ID NO: 40 or a conservative modification thereof, and a CDR3 comprising the amino acid sequence set forth in SEQ ID NO: 41 or a conservative modification thereof. SEQ ID NOs: 39-41 are provided in Table 11.

In certain embodiments, the anti-HPV scFv comprises a V_{H} that comprises a CDR1 comprising the amino acid sequence set forth in SEQ ID NO: 36 or a conservative modification thereof, a CDR2 comprising the amino acid sequence set forth in SEQ ID NO: 37 or a conservative modification thereof, and a CDR3 comprising the amino acid sequence set forth in SEQ ID NO: 38 or a conservative modification thereof; and a V_{L} that comprises a CDR1 comprising the amino acid sequence set forth in SEQ ID NO: 39 or a conservative modification thereof, a CDR2 comprising the amino acid sequence set forth in SEQ ID NO: 40 or a conservative modification, and a CDR3 comprising the amino acid sequence set forth in SEQ ID NO: 41 or a conservative modification thereof.

In certain embodiments, the anti-HPV scFv comprises a V_{H} that comprises a CDR1 comprising the amino acid sequence set forth in SEQ ID NO: 36, a CDR2 comprising the amino acid sequence set forth in SEQ ID NO: 37, and a CDR3 comprising the amino acid sequence set forth in SEQ ID NO: 38; and a V_{L} that comprises a CDR1 comprising the amino acid sequence set forth in SEQ ID NO: 39, a CDR2 comprising the amino acid sequence set forth in SEQ ID NO: 40, and a CDR3 comprising the amino acid sequence set forth in SEQ ID NO: 41.

In certain embodiments, the anti-HPV scFv comprises a V_{H} comprising the amino acid sequence set forth in SEQ ID NO: 42, and a V_{L} comprising the amino acid sequence set forth in SEQ ID NO: 43. In certain embodiments, the V_{H} and V_{L} are linked via a linker. In certain embodiments, the linker comprises or consists of the amino acid sequence set forth in SEQ ID NO: 44, SEQ ID NO: 45, SEQ ID NO: 46, SEQ ID NO: 47, SEQ ID NO: 48, or SEQ ID NO: 49. In certain embodiments, the variable regions are linked one after another such that a heavy chain variable region (V_{H}) is position at the N-terminus. In certain embodiments, the variable regions are positioned from the N- to the C-terminus: V_{H}—V_{L}. In certain embodiments, a light chain variable region (V_{L}) is positioned at the N-terminus. In certain embodiments, the variable regions are positioned from the N- to the C-terminus: V_{L}-V_{H}.

TABLE 11

| CDRs | 1 | 2 | 3 |
|---|---|---|---|
| V_{H} | GFTFSSTA [SEQ ID | ISGSGSYT [SEQ ID | ARHHPDDYGDYVRDAF |
| | NO: 36] | NO: 37] | DI [SEQ ID NO: 38] |
| V_{L} | QSVSHSY [SEQ ID NO: | GAS | QQYEEYPLT [SEQ ID |
| | 39] | [SEQ ID NO: 40] | NO: 41] |

| | |
|---|---|
| Full V_{H} | EVQLLESGGGLVQPGGSLRLSCAASGFTFSSTAMSWVRQAPGKGLEWVSEISGSGSYTS |
| | YADSVKGRFTISRDNSKNTLYLQMNSLRAEDTAVYYCARHHPDDYGDYVRDAFDIWGQG |
| | TMVTVSS [SEQ ID NO: 42] |
| Full V_{L} | EIVLTQSPGTLSLSPGERATLSCRASQSVSHSYLAWYQQKPGQAPRLLIYGASS |
| | RATGIPDRFSGSGSGTDETLTISRLEPEDFAVYYCQQYEEYPLTFGQGTKVEIK |
| | [SEQ ID NO: 43] |
| DNA for | GAAGTGCAGCTGCTGGAATCTGGCGGAGGACTGGTTCAACCTGGCGGCTCTCTG |
| Full V_{H} | AGACTGTCTTGTGCCGCCAGCGGCTTCACCTTTAGCAGCACAGCCATGAGCTGG |
| | GTCCGACAGGCTCCTGGCAAAGGCCTTGAATGGGTGTCCGAGATCAGCGGCAGC |
| | GGCTCTTACACAAGCTACGCCGATAGCGTGAAGGGCAGATTCACCATCAGCCGG |
| | GACAACAGCAAGAACACCCTGTACCTGCAGATGAACAGCCTGAGAGCCGAGGAC |
| | ACCGCCGTGTACTACTGTGCCAGACATCACCCCGACGACTACGGCGATTATGTG |
| | CGGGACGCCTTCGATATCTGGGGCCAGGGAACAATGGTCACCGTGTCTAGT |
| | [SEQ ID NO: 56] |
| DNA for | GAGATCGTGCTGACACAGAGCCCTGGCACACTGTCACTGTCTCCAGGCGAAAGA |
| Full V_{L} | GCCACACTGAGCTGTAGAGCCAGCCAGAGCGTGTCCCACTCTTACCTGGCTTGG |
| | TATCAGCAGAAGCCCGGACAGGCTCCCAGACTGCTGATCTATGGCGCCTCTTCT |
| | AGAGCCACAGGCATCCCCGATAGATTCAGCGGCTCTGGCAGCGGCACCGATTTC |
| | ACCCTGACAATCAGCAGACTGGAACCCGAGGACTTCGCCGTGTACTACTGCCAG |
| | CAGTACGAGGAATACCCTCTGACCTTTGGCCAGGGCACCAAGGTGGAAATCAAG |
| | [SEQ ID NO: 57] |

### 5.3.2. Monoclonal Antibodies

The presently disclosed subject matter provides antibodies (e.g., human antibodies, e.g., human monoclonal antibodies) that specifically bind to HPV (e.g., an HPV/HLA complex, more specifically, a HPV/HLA class I complex, more specifically, a HPV/HLA-A complex, more specifically, a HPV/HLA-A2 complex, or more specifically, a HPV/HLA-A*02:01 complex). The V_{H} amino acid sequences of anti-HPV antibodies 3F8, P1-13, P1-75, P1-80, P2-75-1, P2-75-5, P3-30, P4-10, R4-D1, 2A5, and 1B1 are set forth in SEQ ID NOs: 9, 34, and 42. The V_{L} amino acid sequences of 3F8, P1-13, P1-75, P1-80, P2-75-1, P2-75-5, P3-30, P4-10, R4-D1, 2A5, and 1B1 are set forth in SEQ ID NOs: 10, 12, 15, 17, 20, 22, 24, 26, 28, 35, and 43.

Given that each of 3F8, P1-13, P1-75, P1-80, P2-75-1, P2-75-5, P3-30, P4-10, R4-D1, 2A5, and 1B1 antibodies can bind to HPV, the V_{H} and V_{L} sequences can be “mixed and matched” to create other anti-HPV binding molecules. Binding of such “mixed and matched” antibodies to HPV (e.g., an HPV/HLA complex, more specifically, a HPV/HLA class I complex, more specifically, a HPV/HLA-A complex, more specifically, a HPV/HLA-A2 complex, or more specifically, a HPV/HLA-A*02:01 complex) can be tested using the binding assays known in the art, including for example, ELISAs, Western blots, RIAs, Biacore analysis. Preferably, when V_{H} and V_{L} chains are mixed and matched, a V_{H} sequence from a particular V_{H}/V_{L} pairing is replaced with a structurally similar V_{H} sequence. Likewise, a V_{L} sequence from a particular V_{H}/V_{L} pairing is replaced with a structurally similar V_{L} sequence.

In certain embodiments, the presently disclosed subject matter provides an antibody or an antigen-binding fragment thereof comprising: (a) a heavy chain variable region (V_{H}) comprising an amino acid sequence selected from SEQ ID NOs: 9, 34, and 42; and (b) a light chain variable region (V_{L}) comprising an amino acid sequence selected from SEQ ID NOs: 10, 12, 15, 17, 20, 22, 24, 26, 28, 35, and 43; wherein the antibody or antigen-binding fragment specifically binds to HPV, e.g., an HPV/HLA complex, more specifically, a HPV/HLA class I complex, more specifically, a HPV/HLA-A complex, more specifically, a HPV/HLA-A2 complex, or more specifically, a HPV/HLA-A*02:01 complex. In certain embodiments, the V_{H} and V_{L} are selected from the group consisting of:
(a) a heavy chain variable region comprising the amino acid sequence set forth in SEQ ID NO: 9, and a light chain variable region comprising the amino acid sequence set forth in SEQ ID NO: 10;
(b) a heavy chain variable region comprising the amino acid sequence set forth in SEQ ID NO: 9, and a light chain variable region comprising the amino acid sequence set forth in SEQ ID NO: 12;
(c) a heavy chain variable region comprising the amino acid sequence set forth in SEQ ID NO: 9, and a light chain variable region comprising the amino acid sequence set forth in SEQ ID NO: 15;
(d) a heavy chain variable region comprising the amino acid sequence set forth in SEQ ID NO: 9, and a light chain variable region comprising the amino acid sequence set forth in SEQ ID NO: 17;
(e) a heavy chain variable region comprising the amino acid sequence set forth in SEQ ID NO: 9, and a light chain variable region comprising the amino acid sequence set forth in SEQ ID NO: 20;
(f) a heavy chain variable region comprising the amino acid sequence set forth in SEQ ID NO: 9, and a light chain variable region comprising the amino acid sequence set forth in SEQ ID NO: 22;
(g) a heavy chain variable region comprising the amino acid sequence set forth in SEQ ID NO: 9, and a light chain variable region comprising the amino acid sequence set forth in SEQ ID NO: 24;
(h) a heavy chain variable region comprising the amino acid sequence set forth in SEQ ID NO: 9, and a light chain variable region comprising the amino acid sequence set forth in SEQ ID NO: 26;
(i) a heavy chain variable region comprising the amino acid sequence set forth in SEQ ID NO: 9, and a light chain variable region comprising the amino acid sequence set forth in SEQ ID NO: 28;
(j) a heavy chain variable region comprising the amino acid sequence set forth in SEQ ID NO: 34, and a light chain variable region comprising the amino acid sequence set forth in SEQ ID NO: 35; and
(k) a heavy chain variable region comprising the amino acid sequence set forth in SEQ ID NO: 42, and a light chain variable region comprising the amino acid sequence set forth in SEQ ID NO: 43.

In certain embodiments, the presently disclosed subject matter provides antibodies or antigen-binding fragments thereof that comprise the heavy chain and light chain CDR1s, CDR2s and CDR3s of 3F8, P1-13, P1-75, P1-80, P2-75-1, P2-75-5, P3-30, P4-10, R4-D1, 2A5, and 1B1.

The amino acid sequences of the V_{H} CDR1s of 3F8, P1-13, P1-75, P1-80, P2-75-1, P2-75-5, P3-30, P4-10, R4-D1, 2A5, and 1B1 are set forth in SEQ ID NOs: 3 and 36. The amino acid sequences of the V_{H} CDR2s of 3F8, P1-13, P1-75, P1-80, P2-75-1, P2-75-5, P3-30, P4-10, R4-D1, 2A5, and 1B1 antibodies are set forth in SEQ ID NOs: 4, 29, and 37. The amino acid sequences of the V_{H} CDR3s of 3F8, P1-13, P1-75, P1-80, P2-75-1, P2-75-5, P3-30, P4-10, R4-D1, 2A5, and 1B1 are set forth in SEQ ID NOs: 5, 30, and 38.

The amino acid sequences of the V_{L} CDR1s of 3F8, P1-13, P1-75, P1-80, P2-75-1, P2-75-5, P3-30, P4-10, R4-D1, 2A5, and 1B1 are set forth in SEQ ID NOs: 6, 13, 18, 31, and 39. The amino acid sequences of the V_{L} CDR2s of 3F8, P1-13, P1-75, P1-80, P2-75-1, P2-75-5, P3-30, P4-10, R4-D1, 2A5, and 1B1 are set forth in SEQ ID NOs: 7, 32, and 40. The amino acid sequences of the V_{L} CDR3s of 3F8, P1-13, P1-75, P1-80, P2-75-1, P2-75-5, P3-30, P4-10, R4-D1, 2A5, and 1B1 are set forth in SEQ ID NOs: 8, 11, 14, 16, 19, 21, 23, 25, 27, 33, and 41. The CDR regions are delineated using the IMGT system. In certain embodiments, the CDR regions are delineated using the IMGT numbering system accessible at http://www.imgt.org/IMGT_vquest/input.

Given that each of these antibodies or antigen-binding fragments thereof can bind to HPV (e.g., an HPV/HLA complex, more specifically, a HPV/HLA class I complex, more specifically, a HPV/HLA-A complex, more specifically, a HPV/HLA-A2 complex, or more specifically, a HPV/HLA-A*02:01 complex) and that antigen-binding specificity is provided primarily by the CDR1, CDR2, and CDR3 regions, the V_{H} CDR1, CDR2, and CDR3 sequences and V_{L} CDR1, CDR2, and CDR3 sequences can be “mixed and matched” (i.e., CDRs from different antibodies can be mixed and match, although each antibody must contain a V_{H} CDR1, CDR2, and CDR3 and a V_{L} CDR1, CDR2, and CDR3) to create other anti-HPV binding molecules. HPV binding of such “mixed and matched” antibodies can be tested using the binding assays described above. When V_{H} CDR sequences are mixed and matched, the CDR1, CDR2 and/or CDR3 sequence from a particular V_{H} sequence is replaced with a structurally similar CDR sequence(s). Likewise, when V_{L} CDR sequences are mixed and matched, the CDR1, CDR2 and/or CDR3 sequence from a particular V_{L} sequence preferably is replaced with a structurally similar CDR sequence(s). It will be readily apparent to the ordinarily skilled artisan that novel V_{H} and V_{L} sequences can be created by substituting one or more V_{H} and/or V_{L} CDR region sequences with structurally similar sequences from the CDR sequences of the antibodies or antigen-binding fragments thereof disclosed herein 3F8, P1-13, P1-75, P1-80, P2-75-1, P2-75-5, P3-30, P4-10, R4-D1, 2A5, and 1B1.

In certain embodiments, the presently disclosed subject matter provides an antibody or an antigen-binding fragment thereof comprising:
(a) a heavy chain variable region CDR1 comprising the amino acid sequence set forth in SEQ ID NO: 3, or SEQ ID NO: 36;
(b) a heavy chain variable region CDR2 comprising the amino acid sequence set forth in SEQ ID NO: 4, SEQ ID NO: 29, or SEQ ID NO: 37;
(c) a heavy chain variable region CDR3 comprising the amino acid sequence set forth in SEQ ID NO: 5, SEQ ID NO: 30, or SEQ ID NO: 38;
(d) a light chain variable region CDR1 comprising the amino acid sequence set forth in SEQ ID NO: 6, SEQ ID NO: 13, SEQ ID NO: 18, SEQ ID NO: 31, or SEQ ID NO: 39;
(e) a light chain variable region CDR2 comprising the amino acid sequence set forth in SEQ ID NO: 7, SEQ ID NO: 32, or SEQ ID NO: 40; and
(f) a light chain variable region CDR3 comprising the amino acid sequence set forth in SEQ ID NO: 8, SEQ ID NO: 11, SEQ ID NO: 14, SEQ ID NO: 16, SEQ ID NO: 19, SEQ ID NO: 21, SEQ ID NO: 23, SEQ ID NO: 25, SEQ ID NO: 27, SEQ ID NO: 33, or SEQ ID NO: 41.

In certain embodiments, the antibody or antigen-binding fragment thereof comprises:
(a) a heavy chain variable region CDR1 comprising the amino acid sequence set forth in SEQ ID NO: 3;
(b) a heavy chain variable region CDR2 comprising the amino acid sequence set forth in SEQ ID NO: 4;
(c) a heavy chain variable region CDR3 comprising the amino acid sequence set forth in SEQ ID NO: 5;
(d) a light chain variable region CDR1 comprising the amino acid sequence set forth in SEQ ID NO: 6;
(e) a light chain variable region CDR2 comprising the amino acid sequence set forth in SEQ ID NO: 7; and
(f) a light chain variable region CDR3 comprising the amino acid sequence set forth in SEQ ID NO: 8.

In certain embodiments, the antibody or antigen-binding fragment thereof comprises:
(a) a heavy chain variable region CDR1 comprising the amino acid sequence set forth in SEQ ID NO: 3;
(b) a heavy chain variable region CDR2 comprising the amino acid sequence set forth in SEQ ID NO: 4;
(c) a heavy chain variable region CDR3 comprising the amino acid sequence set forth in SEQ ID NO: 5;
(d) a light chain variable region CDR1 comprising the amino acid sequence set forth in SEQ ID NO: 6;
(e) a light chain variable region CDR2 comprising the amino acid sequence set forth in SEQ ID NO: 7; and
(f) a light chain variable region CDR3 comprising the amino acid sequence set forth in SEQ ID NO: 11.

In certain embodiments, the antibody or antigen-binding fragment thereof comprises:
(a) a heavy chain variable region CDR1 comprising the amino acid sequence set forth in SEQ ID NO: 3;
(b) a heavy chain variable region CDR2 comprising the amino acid sequence set forth in SEQ ID NO: 4;
(c) a heavy chain variable region CDR3 comprising the amino acid sequence set forth in SEQ ID NO: 5;
(d) a light chain variable region CDR1 comprising the amino acid sequence set forth in SEQ ID NO: 13;
(e) a light chain variable region CDR2 comprising the amino acid sequence set forth in SEQ ID NO: 7; and
(f) a light chain variable region CDR3 comprising the amino acid sequence set forth in SEQ ID NO: 14.

In certain embodiments, the antibody or antigen-binding fragment thereof comprises:
(a) a heavy chain variable region CDR1 comprising the amino acid sequence set forth in SEQ ID NO: 3;
(b) a heavy chain variable region CDR2 comprising the amino acid sequence set forth in SEQ ID NO: 4;
(c) a heavy chain variable region CDR3 comprising the amino acid sequence set forth in SEQ ID NO: 5;
(d) a light chain variable region CDR1 comprising the amino acid sequence set forth in SEQ ID NO: 6;
(e) a light chain variable region CDR2 comprising the amino acid sequence set forth in SEQ ID NO: 7; and
(f) a light chain variable region CDR3 comprising the amino acid sequence set forth in SEQ ID NO: 16.

In certain embodiments, the antibody or antigen-binding fragment thereof comprises:
(a) a heavy chain variable region CDR1 comprising the amino acid sequence set forth in SEQ ID NO: 3;
(b) a heavy chain variable region CDR2 comprising the amino acid sequence set forth in SEQ ID NO: 4;
(c) a heavy chain variable region CDR3 comprising the amino acid sequence set forth in SEQ ID NO: 5;
(d) a light chain variable region CDR1 comprising the amino acid sequence set forth in SEQ ID NO: 18;
(e) a light chain variable region CDR2 comprising the amino acid sequence set forth in SEQ ID NO: 7; and
(f) a light chain variable region CDR3 comprising the amino acid sequence set forth in SEQ ID NO: 19.

In certain embodiments, the antibody or antigen-binding fragment thereof comprises:
(a) a heavy chain variable region CDR1 comprising the amino acid sequence set forth in SEQ ID NO: 3;
(b) a heavy chain variable region CDR2 comprising the amino acid sequence set forth in SEQ ID NO: 4;
(c) a heavy chain variable region CDR3 comprising the amino acid sequence set forth in SEQ ID NO: 5;
(d) a light chain variable region CDR1 comprising the amino acid sequence set forth in SEQ ID NO: 6;
(e) a light chain variable region CDR2 comprising the amino acid sequence set forth in SEQ ID NO: 7; and
(f) a light chain variable region CDR3 comprising the amino acid sequence set forth in SEQ ID NO: 21.

In certain embodiments, the antibody or antigen-binding fragment thereof comprises:
(a) a heavy chain variable region CDR1 comprising the amino acid sequence set forth in SEQ ID NO: 3;
(b) a heavy chain variable region CDR2 comprising the amino acid sequence set forth in SEQ ID NO: 4;
(c) a heavy chain variable region CDR3 comprising the amino acid sequence set forth in SEQ ID NO: 5;
(d) a light chain variable region CDR1 comprising the amino acid sequence set forth in SEQ ID NO: 6;
(e) a light chain variable region CDR2 comprising the amino acid sequence set forth in SEQ ID NO: 7; and
(f) a light chain variable region CDR3 comprising the amino acid sequence set forth in SEQ ID NO: 23.

In certain embodiments, the antibody or antigen-binding fragment thereof comprises:
(a) a heavy chain variable region CDR1 comprising the amino acid sequence set forth in SEQ ID NO: 3;
(b) a heavy chain variable region CDR2 comprising the amino acid sequence set forth in SEQ ID NO: 4;
(c) a heavy chain variable region CDR3 comprising the amino acid sequence set forth in SEQ ID NO: 5;
(d) a light chain variable region CDR1 comprising the amino acid sequence set forth in SEQ ID NO: 6;
(e) a light chain variable region CDR2 comprising the amino acid sequence set forth in SEQ ID NO: 7; and
(f) a light chain variable region CDR3 comprising the amino acid sequence set forth in SEQ ID NO: 25.

In certain embodiments, the antibody or antigen-binding fragment thereof comprises:
(a) a heavy chain variable region CDR1 comprising the amino acid sequence set forth in SEQ ID NO: 3;
(b) a heavy chain variable region CDR2 comprising the amino acid sequence set forth in SEQ ID NO: 4;
(c) a heavy chain variable region CDR3 comprising the amino acid sequence set forth in SEQ ID NO: 5;
(d) a light chain variable region CDR1 comprising the amino acid sequence set forth in SEQ ID NO: 6;
(e) a light chain variable region CDR2 comprising the amino acid sequence set forth in SEQ ID NO: 7; and
(f) a light chain variable region CDR3 comprising the amino acid sequence set forth in SEQ ID NO: 27.

In certain embodiments, the antibody or antigen-binding fragment thereof comprises:
(a) a heavy chain variable region CDR1 comprising the amino acid sequence set forth in SEQ ID NO: 3;
(b) a heavy chain variable region CDR2 comprising the amino acid sequence set forth in SEQ ID NO: 29;
(c) a heavy chain variable region CDR3 comprising the amino acid sequence set forth in SEQ ID NO: 30;
(d) a light chain variable region CDR1 comprising the amino acid sequence set forth in SEQ ID NO: 31;
(e) a light chain variable region CDR2 comprising the amino acid sequence set forth in SEQ ID NO: 32; and
(f) a light chain variable region CDR3 comprising the amino acid sequence set forth in SEQ ID NO: 33.

In certain embodiments, the antibody or antigen-binding fragment thereof comprises:
(a) a heavy chain variable region CDR1 comprising the amino acid sequence set forth in SEQ ID NO: 36;
(b) a heavy chain variable region CDR2 comprising the amino acid sequence set forth in SEQ ID NO: 37;
(c) a heavy chain variable region CDR3 comprising the amino acid sequence set forth in SEQ ID NO: 38;
(d) a light chain variable region CDR1 comprising the amino acid sequence set forth in SEQ ID NO: 39;
(e) a light chain variable region CDR2 comprising the amino acid sequence set forth in SEQ ID NO: 40; and
(f) a light chain variable region CDR3 comprising the amino acid sequence set forth in SEQ ID NO: 41.

In certain embodiments, the anti-HPV antibody or antigen-binding fragment thereof comprises a heavy chain constant region and/or a light chain constant region.

In certain embodiments, the heavy chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 50. In certain embodiments, the heavy chain constant region comprises the amino acid sequence set forth in SEQ ID NO: 50. SEQ ID NO: 50 is provided below.

[SEQ ID NO: 50]
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK

In certain embodiments, the light chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 51. In certain embodiments, the light chain constant region comprises the amino acid sequence set forth in SEQ ID NO: 51. SEQ ID NO: 51 is provided below.

[SEQ ID NO: 51]
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC

In certain embodiments, the anti-HPV antibody or an antigen-binding fragment thereof comprises: (a) a heavy chain constant region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 50; and (b) a light chain constant region comprising an amino acid sequence that is at least about 80%, at least about 85%, at least about 90%, at least about 95%, at least about 96%, at least about 97%, at least about 98%, at least about 99% at least about 100% homologous or identical to the amino acid sequence set forth in SEQ ID NO: 51. In certain embodiments, the anti-HPV antibody or an antigen-binding fragment thereof comprises: (a) a heavy chain constant region comprising the amino acid sequence set forth in SEQ ID NO: 50; and (b) a light chain constant region comprising the amino acid sequence set forth in SEQ ID NO: 51.

The constant regions/framework regions of the anti-HPV antibodies disclosed herein can be altered, for example, by amino acid substitution, to modify the properties of the antibody (e.g., to increase or decrease one or more of: antigen binding affinity, Fc receptor binding, antibody carbohydrate, for example, glycosylation, fucosylation etc., the number of cysteine residues, effector cell function, effector cell function, complement function or introduction of a conjugation site).

In certain embodiments, a presently disclosed anti-HPV antibody is a fully-human antibody, e.g., any one of 3F8, P1-13, P1-75, P1-80, P2-75-1, P2-75-5, P3-30, P4-10, R4-D1, 2A5, and 1B1.

Fully-human mAbs, when administered to humans, causing serious side effects, including anaphylaxis and hypersensitivity reactions.

The use of phage display libraries has made it possible to select large numbers of antibody repertoires for unique and rare Abs against very defined epitopes (for more details on phage display see McCafferty et al., Phage antibodies: filamentous phage displaying antibody variable domains. Nature, 348: 552-554.) The rapid identification of human Fab or single chain Fv (scFv) fragments highly specific for tumor antigen-derived peptide-MHC complex molecules has thus become possible. In addition, by engineering full-length monoclonal antibody (mAb) using the Fab fragments, it is possible to directly generate a therapeutic human mAb, bypassing months of time-consuming work, normally needed for developing therapeutic mAbs. The presently disclosed subject matter involves the development of a fully human mAb that recognizes, for example, a HPV polypeptide (e.g., an HPV/HLA complex, more specifically, a HPV/HLA class I complex, more specifically, a HPV/HLA-A complex, more specifically, a HPV/HLA-A2 complex, or more specifically, a HPV/HLA-A*02:01 complex) for cancer therapy.

### 5.3.3. Homologous Antibodies

In certain embodiments, a presently disclosed anti-HPV antibody or antigen-binding fragment thereof comprises heavy and light chain variable regions comprising amino acid sequences that are homologous or identical to the amino acid sequences of the antibodies described herein (e.g., 3F8, P1-13, P1-75, P1-80, P2-75-1, P2-75-5, P3-30, P4-10, R4-D1, 2A5, and 1B1 antibodies), and wherein the antibodies or antigen-binding fragments thereof retain the desired functional properties of the anti-HPV antibodies or antigen-binding fragments thereof of the presently disclosed subject matter.

For example, the presently disclosed subject matter provides an anti-HPV antibody or an antigen-binding fragment thereof, comprising a heavy chain variable region and a light chain variable region, wherein:
(a) the heavy chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 9, SEQ ID NO: 34, or SEQ ID NO: 42; and
(b) the light chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 10, SEQ ID NO: 12, SEQ ID NO: 15, SEQ ID NO: 17, SEQ ID NO: 20, SEQ ID NO: 22, SEQ ID NO: 24, SEQ ID NO: 26, SEQ ID NO: 28, SEQ ID NO: 35, or SEQ ID NO: 43.

In certain embodiments, the V_{H} and/or V_{L} amino acid sequences can be at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the sequences set forth above. An antibody having V_{H} and V_{L} regions having high (i.e., 80% or greater) homology or identity to the V_{H} and V_{L} regions of the sequences set forth above, can be obtained by mutagenesis (e.g., site-directed or PCR-mediated mutagenesis), followed by testing of the encoded altered antibody for retained function (i.e., the binding affinity) using the binding assays described herein.

In certain embodiments, a presently disclosed anti-HPV antibody or antigen-binding fragment thereof comprises heavy and light chain constant regions comprising amino acid sequences that are homologous or identical to the amino acid sequences of the antibodies described herein (e.g., 3F8, P1-13, P1-75, P1-80, P2-75-1, P2-75-5, P3-30, P4-10, R4-D1, 2A5, and 1B1 antibodies), and wherein the antibodies or antigen-binding fragments thereof retain the desired functional properties of the anti-HPV antibodies or antigen-binding fragments thereof of the presently disclosed subject matter.

For example, the presently disclosed subject matter provides an anti-HPV antibody or an antigen-binding fragment thereof, comprising a heavy chain constant region and a light chain constant region, wherein:
(a) the heavy chain constant region (C_{H}) comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 50; and
(b) the light chain constant region (C_{L}) comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 51.

In certain embodiments, the C_{H} and/or C_{L} amino acid sequences can be at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the sequences set forth above. An antibody having C_{H} and C_{L} regions having high (i.e., 80% or greater) homology or identity to the C_{H} and C_{L} regions of the sequences set forth above, can be obtained by mutagenesis (e.g., site-directed or PCR-mediated mutagenesis), followed by testing of the encoded altered antibody for retained function (i.e., the binding affinity) using the binding assays described herein.

As used herein, the percent homology between two amino acid sequences is equivalent to the percent identity between the two sequences. The percent identity or homology between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent homology or identity between two amino acid sequences can be determined using the algorithm of E. Meyers and W. Miller (Comput Appl Biosci (1988); 14:11-17) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J Mol Biol (1970); 48:444-453) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the presently disclosed subject matter can further be used as a “query sequence” to perform a search against public databases to, for example, identify related sequences. Such searches can be performed using the XBLAST program (version 2.0) of Altschul et al., J Mol Biol (1990); 215:403-10. BLAST protein searches can be performed with the XBLAST program, score=50, wordlength=3 to obtain amino acid sequences homologous to the antibody molecules of the invention. To obtain gapped alignments for comparison purposes, Gapped BLAST can be utilized as described in Altschul et al., Nucleic Acids Res (1997); 25(17):3389-3402. When utilizing BLAST and Gapped BLAST programs, the default parameters of the respective programs (e.g., XBLAST and NBLAST) can be used.

### 5.3.4. Antibodies with Conservative Modifications

In certain embodiments, a presently disclosed anti-HPV antibody or an antigen-binding fragment thereof comprises a heavy chain variable region comprising CDR1, CDR2 and CDR3 sequences and a light chain variable region comprising CDR1, CDR2 and CDR3 sequences, wherein one or more of these CDR sequences comprise specified amino acid sequences based on the preferred antibodies described herein (e.g., 3F8, P1-13, P1-75, P1-80, P2-75-1, P2-75-5, P3-30, P4-10, R4-D1, 2A5, and 1B1 antibodies), or a conservative modification thereof, and wherein the antibodies retain the desired functional properties of the anti-HPV antibodies or antigen-binding fragments thereof of the presently disclosed subject matter. The presently disclosed subject matter provides an antibody or an antigen-binding fragment thereof, comprising a heavy chain variable region comprising CDR1, CDR2, and CDR3 sequences and a light chain variable region comprising CDR1, CDR2, and CDR3 sequences, wherein:
(a) the heavy chain variable region CDR3 sequence comprises an amino acid sequence selected from SEQ ID NOs: 5, 30, and 38, and conservative modifications thereof;
(b) the light chain variable region CDR3 sequence comprises an amino acid sequence selected from SEQ ID NOs: 8, 11, 14, 16, 19, 21, 23, 25, 27, 33, and 41, and conservative modifications thereof.

In certain embodiments, the heavy chain variable region CDR3 sequence comprises an amino acid sequence selected from SEQ ID NOs: 5, 30, and 38, and conservative modifications thereof; and the light chain variable region CDR3 sequence comprises an amino acid sequence selected from SEQ ID NOs: 8, 11, 14, 16, 19, 21, 23, 25, 27, 33, and 41, and conservative modifications thereof.

In certain embodiments, the heavy chain variable region CDR2 sequence comprises an amino acid sequence selected from SEQ ID NOs: 4, 29, and 37, and conservative modifications thereof; and the light chain variable region CDR2 sequence comprises an amino acid sequence selected from SEQ ID NOs: 7, 32, and 40, and conservative modifications thereof.

In certain embodiments, the heavy chain variable region CDR1 sequence comprises an amino acid sequence selected from SEQ ID NOs: 3 and 36, and conservative modifications thereof; and the light chain variable region CDR1 sequence comprises an amino acid sequence selected from SEQ ID NOs: 6, 13, 18, 31, and 39, and conservative modifications thereof.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the present disclosure by standard techniques known in the art, such as site-directed mutagenesis and PCR-mediated mutagenesis.

Conservative amino acid substitutions are ones in which the amino acid residue is replaced with an amino acid residue having a similar side chain. Families of amino acid residues having similar side chains have been defined in the art. Exemplary conservative amino acid substitutions are shown in Table 12. Amino acid substitutions may be introduced into an antibody of interest and the products screened for a desired activity, e.g., retained/improved antigen binding, decreased immunogenicity, or improved ADCC or CDC. In certain embodiments, a sequence disclosed herein, e.g., a CDR sequence, a V_{H} sequence or a V_{L} sequence, can have up to about one, up to about two, up to about three, up to about four, up to about five, up to about six, up to about seven, up to about eight, up to about nine or up to about ten amino acid residues that are modified and/or substituted.

TABLE 12

| Original Residue | Exemplary conservative amino acid Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe |
| Leu (L) | Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala |

Amino acids may be grouped according to common side-chain properties:
hydrophobic: Norleucine, Met, Ala, Val, Leu, Ile;
neutral hydrophilic: Cys, Ser, Thr, Asn, Gln;
acidic: Asp, Glu;
basic: His, Lys, Arg;
residues that influence chain orientation: Gly, Pro;
aromatic: Trp, Tyr, Phe.

Non-conservative substitutions will entail exchanging a member of one of these classes for another class.

5.3.5. Anti-HPV Antibodies that Cross-Compete for Binding to HPV with Anti-HPV Antibodies of the Invention

The presently disclosed subject matter provides antibodies or antigen-binding fragments thereof that cross-compete with any of the disclosed anti-HPV antibodies for binding to HPV/MHC (e.g., an HPV/HLA complex, more specifically, a HPV/HLA class I complex, more specifically, a HPV/HLA-A complex, more specifically, a HPV/HLA-A2 complex, or more specifically, a HPV/HLA-A*02:01 complex). For example, and not by way of limitation, the cross-competing antibodies can bind to the same epitope region, e.g., same epitope, adjacent epitope, or overlapping as any of the anti-HPV antibodies or antigen-binding fragments thereof of the presently disclosed subject matter. In certain embodiments, the reference antibody or reference antigen-binding fragments thereof for cross-competition studies can be any one of the anti-HPV antibodies or antigen-binding fragments thereof disclosed herein, e.g., 3F8, P1-13, P1-75, P1-80, P2-75-1, P2-75-5, P3-30, P4-10, R4-D1, 2A5, and 1B1 antibodies.

Such cross-competing antibodies can be identified based on their ability to cross-compete with any one of the presently disclosed anti-HPV antibodies or antigen-binding fragments thereof in standard HPV binding assays. For example, Biacore analysis, ELISA assays or flow cytometry can be used to demonstrate cross-competition with the antibodies of the presently disclosed subject matter. The ability of a test antibody to inhibit the binding of, for example, any one of the presently disclosed anti-HPV antibodies (e.g., 3F8, P1-13, P1-75, P1-80, P2-75-1, P2-75-5, P3-30, P4-10, R4-D1, 2A5, and 1B1 antibodies) to HPV (e.g., HPV/MHC complex) demonstrates that the test antibody can compete with any one of the presently disclosed anti-HPV antibodies or antigen-binding fragments thereof for binding to HPV (e.g., HPV/MHC complex) and thus binds to the same epitope region on HPV (e.g., HPV/MHC complex) as any one of the presently disclosed anti-HPV antibodies or antigen-binding fragments thereof. In certain embodiments, the cross-competing antibody or antigen-binding fragment thereof binds to the same epitope on HPV (e.g., HPV/MHC complex) as any one of the presently disclosed anti-HPV antibodies or antigen-binding fragments thereof.

### 5.3.6. Characterization of Antibody Binding to Antigen

Antibodies or antigen-binding fragments thereof of the presently disclosed subject can be tested for binding to HPV by, for example, standard ELISA. To determine if the selected anti-HPV antibodies bind to unique epitopes, each antibody can be biotinylated using commercially available reagents (Pierce, Rockford, IL). Competition studies using unlabeled monoclonal antibodies and biotinylated monoclonal antibodies can be performed using HPV coated-ELISA plates as described above. Biotinylated mAb binding can be detected with a strep-avidin-alkaline phosphatase probe.

To determine the isotype of purified antibodies, isotype ELISAs can be performed using reagents specific for antibodies of a particular isotype. Anti-HPV human IgGs can be further tested for reactivity with HPV antigen by Western blotting.

In certain embodiments, the K_{D} is measured by a radiolabeled antigen binding assay (RIA). In certain embodiments, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J Mol Biol (1999); 293:865-881).

In certain embodiments, the K_{D} is measured using a BIACORE® surface plasmon resonance assay. For example, an assay using a BIACORE®-2000 or a BIACORE®-3000 (BIAcore, Inc., Piscataway, NJ)

### 5.3.7. Immunoconjugates

The presently disclosed subject provides an anti-HPV antibody or an antigen-binding fragment thereof, conjugated to a therapeutic moiety, such as a cytotoxin, a drug (e.g., an immunosuppressant) or a radiotoxin. Such conjugates are referred to herein as “immunoconjugates”. Immunoconjugates that include one or more cytotoxins are referred to as “immunotoxins.” A cytotoxin or cytotoxic agent includes any agent that is detrimental to (e.g., kills) cells. Non-limiting examples of cytotoxins include plant cytotoxins, bacterial cytotoxins, taxol (such as ricin, diphtheria, gelonin), cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents also include, for example, calecheamicin, aureastatin, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), hypomethylating agents (azacytidine and decitabine), and anti-mitotic agents (e.g., vincristine and vinblastine).

Other examples of therapeutic cytotoxins that can be conjugated to an anti-HPV antibody disclosed herein include duocarmycins, calicheamicins, maytansines and auristatins, and derivatives thereof. Cytotoxins can be conjugated to an anti-HPV antibody or an antigen-binding fragment thereof disclosed herein using linker technology available in the art. Examples of linker types that have been used to conjugate a cytotoxin to an antibody include, but are not limited to, hydrazones, thioethers, esters, disulfides and peptide-containing linkers. A linker can be chosen that is, for example, susceptible to cleavage by low pH within the lysosomal compartment or susceptible to cleavage by proteases, such as proteases preferentially expressed in tumor tissue such as cathepsins (e.g., cathepsins B, C, D). For further discussion of types of cytotoxins, linkers and methods for conjugating therapeutic agents to antibodies, see also Saito, G. et al. (2003) Adv. Drug Deliv. Rev. 55:199-215; Trail, P. A. et al. (2003) Cancer Immunol. Immunother. 52:328-337; Payne, G. (2003) Cancer Cell 3:207-212; Allen, T. M. (2002) Nat. Rev. Cancer 2:750-763; Pastan, I. and Kreitman, R. J. (2002) Curr. Opin. Investig. Drugs 3:1089-1091; Senter, P. D. and Springer, C. J. (2001) Adv. Drug Deliv. Rev. 53:247-264.

Anti-HPV antibodies or antigen-binding fragments thereof of the presently disclosed subject matter also can be conjugated to a radioactive isotope to generate cytotoxic radiopharmaceuticals, also referred to as radioimmunoconjugates. Non-limiting examples of radioactive isotopes that can be conjugated to antibodies for use diagnostically or therapeutically include ⁴⁷Sc, ⁶⁷Cu, ⁹⁰Y, ¹³¹I, ¹⁴⁹Tb, ¹⁶¹Tb ¹⁷⁷Lu, ²²⁵Ac, ²¹³Bi, ²²³Ra and ²²⁷Th. Methods for preparing radioimmunoconjugates are established in the art. Examples of radioimmunoconjugates are commercially available, including Zevalin™ (IDEC Pharmaceuticals) and Bexxar™ (Corixa Pharmaceuticals), and similar methods can be used to prepare radioimmunoconjugates using the presently disclosed anti-HPV antibodies.

The antibody conjugates of the presently disclosed subject matter can be used to modify a given biological response, and the drug moiety is not to be construed as limited to classical chemical therapeutic agents. For example, the drug moiety may be a protein or polypeptide possessing a desired biological activity. Such proteins may include, for example, an enzymatically active toxin, or active fragment thereof, such as abrin, ricin A, Pseudomonas exotoxin, or diphtheria toxin; a protein such as tumor necrosis factor (TNF) or interferon-7; or, biological response modifiers such as, for example, lymphokines, interleukin-1 (IL-1), interleukin-2 (IL-2), interleukin-6 (IL-6), granulocyte macrophage colony stimulating factor (GM-CSF), granulocyte colony stimulating factor (G-CSF), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

### 5.3.8. Multi-Specific Molecules

The presently disclosed subject matter provides multi-specific molecules comprising an anti-HPV antibody, or a fragment thereof, disclosed herein. A presently disclosed or an antigen-binding fragment thereof can be derivatized or linked to one more functional molecules, e.g., one or more peptides or proteins (e.g., one or more antibodies or ligands for a receptor) to generate a multi-specific molecule that binds to two or more different binding sites or target molecules. The presently disclosed anti-HPV antibody or antigen-binding fragment thereof can in fact be derivatized or linked to more than one other functional molecules to generate multi-specific molecules that bind to more than two different binding sites and/or target molecules. To create a multi-specific molecule, a presently disclosed anti-HPV antibody or an antigen-binding fragment thereof can be functionally linked (e.g., by chemical coupling, genetic fusion, noncovalent association or otherwise) to one or more other binding molecules, such as another antibody, antibody fragment, peptide or binding mimetic, such that a bispecific molecule.

In certain embodiments, the multi-specific molecule is a bispecific molecule. In certain embodiments, the bispecific molecules comprises at least a first binding specificity for HPV (e.g., an HPV/HLA complex, more specifically, a HPV/HLA class I complex, more specifically, a HPV/HLA-A complex, more specifically, a HPV/HLA-A2 complex, or more specifically, a HPV/HLA-A*02:01 complex) and a second binding specificity for a second target epitope region. The second target epitope region can be an HPV epitope, or a non-HPV epitope, e.g., a different antigen. In certain embodiments, the multi-specific molecule comprises a first binding specificity for HPV, a second binding specificity for a second target, and a third binding specificity for a third target. In certain embodiments, the second target is an antigen expressed on the surface of an immune cell (e.g., a T cell, or a human immune effector cell). In certain embodiments, the multi-specific molecule is capable of recruiting the activity of that immune effector cell by specifically binding to the effector antigen on the human immune effector cell, thereby enhancing effector function. In certain embodiments, the third target is an antigen expressed on a senescent cell.

The multi-specific molecules of the presently disclosed subject matter can be prepared by conjugating the constituent binding specificities using methods known in the art. For example, each binding specificity of the multi-specific molecule can be generated separately and then conjugated to one another. When the binding specificities are proteins or peptides, a variety of coupling or cross-linking agents can be used for covalent conjugation. Non-limiting examples of cross-linking agents include protein A, carbodiimide, N-succinimidyl-S-acetyl-thioacetate (SATA), 5, 5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl) cyclohaxane-1-carboxylate (sulfo-SMCC) (see e.g., Karpovsky et al. (1984) J. Exp. Med. 160:1686; Liu, M A et al. (1985) Proc. Natl. Acad. Sci. USA 82:8648). Other methods include those described in Paulus (1985) Behring Ins. Mitt. No. 78, 118-132; Brennan et al. (1985) Science 229:81-83), and Glennie et al. (1987) J. Immunol. 139: 2367-2375). Conjugating agents can be SATA and sulfo-SMCC, both available from Pierce Chemical Co. (Rockford, IL).

When the binding specificities are antibodies, they can be conjugated via sulfhydryl bonding of the C-terminus hinge regions of the two heavy chains. In certain embodiments, the hinge region is modified to contain an odd number of sulfhydryl residues, preferably one, prior to conjugation.

Alternatively, both binding specificities can be encoded in the same vector and expressed and assembled in the same host cell. This method is particularly useful where the multi-specific molecule is a mAb x mAb, mAb x Fab, Fab x F(ab′)₂ or ligand x Fab fusion protein.

Binding of the multi-specific molecules to their specific targets can be confirmed by, for example, enzyme-linked immunosorbent assay (ELISA), radioimmunoassay (RIA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest. Alternatively, the complexes can be detected using any of a variety of other immunoassays. For example, the antibody can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography.

### 5.4. Antigen-Recognizing Receptors

The presently disclosed subject matter provides antigen-recognizing receptors. The presently disclosed antigen-recognizing receptors specifically target or bind to HPV (e.g., an HPV/MHC complex). In certain embodiments, the antigen-recognizing receptor is a chimeric antigen receptor (CAR). In certain embodiments, the antigen-recognizing receptor is a TCR like fusion molecule. In certain embodiments, the antigen-recognizing receptor is a T cell receptor fusion construct (TRuC).

### 5.4.1. Chimeric Antigen Receptors (CARs)

In certain embodiments, the antigen-recognizing receptor is a chimeric antigen receptor (CAR). CARs are engineered receptors, which graft or confer a specificity of interest onto an immune effector cell. CARs can be used to graft the specificity of a monoclonal antibody onto a T cell; with transfer of their coding sequence facilitated by retroviral vectors.

There are three generations of CARs. “First generation” CARs are typically composed of an extracellular antigen-binding domain (e.g., an scFv), which is fused to a transmembrane domain, which is fused to cytoplasmic/intracellular signaling domain. “First generation” CARs can provide de novo antigen recognition and cause activation of both CD4⁺ and CD8⁺ T cells through their CD3ζ chain signaling domain in a single fusion molecule, independent of HLA-mediated antigen presentation. “Second generation” CARs add intracellular signaling domains from various co-stimulatory molecules (e.g., CD28, 4-1BB, ICOS, OX40) to the cytoplasmic tail of the CAR to provide additional signals to the T cell. “Second generation” CARs comprise those that provide both co-stimulation (e.g., CD28 or 4-1BB) and activation (CD3ζ). “Third generation” CARs comprise those that provide multiple co-stimulation (e.g., CD28 and 4-1BB) and activation (CD3ζ). In certain embodiments, the antigen-recognizing receptor is a first-generation CAR. In certain embodiments, the antigen-recognizing receptor is a CAR that does not comprise an intracellular signaling domain of a co-stimulatory molecule or a fragment thereof. In certain embodiments, the antigen-recognizing receptor is a second-generation CAR. Additional information regarding CARs and their structure can be found in International Patent Publication No. WO2019133969A2, the content of which is incorporated by reference in its entirety.

In certain embodiments, the CAR comprises an extracellular antigen-binding domain that specifically binds to HPV (e.g., an HPV/MHC complex), a transmembrane domain, and an intracellular signaling domain. The extracellular antigen-binding domain of the CAR can include an scFv disclosed herein, e.g., any one of those disclosed in Section 5.3.1.

In addition, the extracellular antigen-binding domain can comprise a leader or a signal peptide that directs the nascent protein into the endoplasmic reticulum. Signal peptide or leader can be essential if the CAR is to be glycosylated and anchored in the cell membrane. The signal sequence or leader can be a peptide sequence (about 5, about 10, about 15, about 20, about 25, or about 30 amino acids long) present at the N-terminus of newly synthesized proteins that directs their entry to the secretory pathway. In certain embodiments, the signal peptide is covalently joined to the 5′ terminus of the extracellular antigen-binding domain.

In certain non-limiting embodiments, the transmembrane domain of the CAR comprises a hydrophobic alpha helix that spans at least a portion of the membrane. Different transmembrane domains result in different receptor stability. After antigen recognition, receptors cluster and a signal are transmitted to the cell. In accordance with the presently disclosed subject matter, the transmembrane domain of the CAR can comprise a native or modified transmembrane domain of CD8 or a fragment thereof, a native or modified transmembrane domain of CD28 or a fragment thereof, a native or modified transmembrane domain of CD3ζ or a fragment thereof, a native or modified transmembrane domain of CD4 or a fragment thereof, a native or modified transmembrane domain of 4-1BB or a fragment thereof, a native or modified transmembrane domain of OX40 or a fragment thereof, a native or modified transmembrane domain of ICOS or a fragment thereof, a native or modified transmembrane domain of CD84 or a fragment thereof, a native or modified transmembrane domain of CD166 or a fragment thereof, a native or modified transmembrane domain of CD8a or a fragment thereof, a native or modified transmembrane domain of CD8b or a fragment thereof, a native or modified transmembrane domain of ICAM-1 or a fragment thereof, a native or modified transmembrane domain of CTLA-4 or a fragment thereof, a native or modified transmembrane domain of CD27 or a fragment thereof, a native or modified transmembrane domain of CD40 or a fragment thereof, NKGD2 or a fragment thereof, or a combination thereof.

In certain embodiments, the CAR further comprises a spacer region that links the extracellular antigen-binding domain to the transmembrane domain. The spacer region can be flexible enough to allow the antigen binding domain to orient in different directions to facilitate antigen recognition while preserving the activating activity of the CAR.

In certain embodiments, the hinge/spacer region of the CAR comprises a native or modified hinge region of CD8 or a fragment thereof, a native or modified hinge region of CD28 or a fragment thereof, a native or modified hinge region of CD3ζ or a fragment thereof, a native or modified hinge region of CD40 or a fragment thereof, a native or modified hinge region of 4-1BB or a fragment thereof, a native or modified hinge region of OX40 or a fragment thereof, a native or modified hinge region of CD84 or a fragment thereof, a native or modified hinge region of CD166 or a fragment thereof, a native or modified hinge region of CD8a or a fragment thereof, a native or modified hinge region of CD8b or a fragment thereof, a native or modified hinge region of ICOS or a fragment thereof, a native or modified hinge region of ICAM-1 or a fragment thereof, a native or modified hinge region of CTLA-4 or a fragment thereof, a native or modified hinge region of CD27 or a fragment thereof, a native or modified hinge region of CD40 or a fragment thereof, a native or modified hinge region of NKGD2 or a fragment thereof, a synthetic polypeptide (not based on a protein associated with the immune response), or a combination thereof. The hinge/spacer region can be the hinge region from IgG1, or the CH₂CH₃ region of immunoglobulin and portions of CD3, a portion of a CD28 polypeptide, a portion of a CD8 polypeptide, a variation of any of the foregoing which is at least about 80%, at least about 85%, at least about 90%, at least about 95%, or at least about 100% homologous or identical thereto, or a synthetic spacer sequence.

In certain embodiments, the CAR comprises an intracellular signaling domain. In certain non-limiting embodiments, the intracellular signaling domain of the CAR comprises a CD3ζ polypeptide. CD3ζ can activate or stimulate a cell (e.g., a cell of the lymphoid lineage, e.g., a T cell). Wild type (“native”) CD3ζ comprises three functional immunoreceptor tyrosine-based activation motifs (ITAMs), three functional basic-rich stretch (BRS) regions (BRS1, BRS2 and BRS3). CD3ζ transmits an activation signal to the cell (e.g., a cell of the lymphoid lineage, e.g., a T cell) after antigen is bound. The intracellular signaling domain of the CD3ζ-chain is the primary transmitter of signals from endogenous TCRs.

In certain embodiments, the intracellular signaling domain of the CAR further comprises at least a co-stimulatory signaling region. In certain embodiments, the co-stimulatory signaling region comprises at least one co-stimulatory molecule or a fragment thereof. In certain embodiments, the co-stimulatory signaling region comprises an intracellular domain of at least one co-stimulatory molecule or a fragment thereof.

As used herein, a “co-stimulatory molecule” refers to a cell surface molecule other than antigen receptor or its ligand that can provide an efficient response of lymphocytes to an antigen. In certain embodiments, a co-stimulatory molecule can provide optimal lymphocyte activation. Non-limiting examples of co-stimulatory molecules include CD28, 4-1BB, OX40, ICOS, DAP-10, CD27, CD40, NKGD2, CD2, FN14, HVEM, LTBR, CD28H, TNFR1, TNFR2, BAFF-R, BCMA, TACI, TROY, RANK, CD40, CD27, CD30, EDAR, XEDAR, GITR, DR6, and NGFR, and combinations thereof. The co-stimulatory molecule can bind to a co-stimulatory ligand, which is a protein expressed on cell surface that upon binding to its receptor produces a co-stimulatory response, i.e., an intracellular response that effects the stimulation provided when an antigen-recognizing receptor (e.g., a chimeric antigen receptor (CAR)) binds to its target antigen. As one example, a 4-1BB ligand (i.e., 4-1BBL) may bind to 4-1BB for providing an intracellular signal that in combination with a CAR signal induces an effector cell function of the CAR⁺ T cell.

In certain embodiments, a presently disclosed CAR further comprises an inducible promoter, for expressing nucleic acid sequences in human cells. Promoters for use in expressing CAR genes can be a constitutive promoter, such as ubiquitin C (UbiC) promoter.

### 5.4.2. TCR Like Fusion Molecule

In certain embodiments, the antigen-recognizing receptor is a TCR like fusion molecule. Non-limiting examples of TCR fusion molecules include HLA-Independent TCR-based Chimeric Antigen Receptor (also known as “HIT”, e.g., those disclosed in International Patent Application No. PCT/US19/017525, which is incorporated by reference in its entirety).

In certain embodiments, the TCR like fusion molecule comprises an antigen binding chain that comprises an extracellular antigen-binding domain and a constant domain, wherein the TCR like fusion molecule binds to an antigen in an HLA-independent manner. In certain embodiments, the constant domain comprises a T cell receptor constant region selected from the group consisting of a native or modified TRAC peptide, a native or modified TRBC peptide, a native or modified TRDC peptide, a native or modified TRGC peptide and any variants or functional fragments thereof. In certain embodiments, the constant domain comprises a native or modified TRAC peptide. In certain embodiments, the constant domain comprises a native or modified TRBC peptide. In certain embodiments, the constant domain is capable of forming a homodimer or a heterodimer with another constant domain. In certain embodiments, the antigen binding chain is capable of associating with a CD3ζ polypeptide. In certain embodiments, the antigen binding chain, upon binding to an antigen, is capable of activating the CD3ζ polypeptide associated to the antigen binding chain. In certain embodiments, the activation of the CD3ζ polypeptide is capable of activating an immunoresponsive cell. In certain embodiments, the TCR like fusion molecule is capable of integrating with a CD3 complex and providing HLA-independent antigen recognition. In certain embodiments, the TCR like fusion molecule replaces an endogenous TCR in a CD3/TCR complex. In certain embodiments, the extracellular antigen-binding domain of the TCR like fusion molecule is capable of dimerizing with another extracellular antigen-binding domain. In certain embodiments, the extracellular antigen-binding domain of the TCR like fusion molecule comprises a ligand for a cell-surface receptor, a receptor for a cell surface ligand, an antigen binding portion of an antibody or a fragment thereof or an antigen binding portion of a TCR. In certain embodiments, the extracellular antigen-binding domain of the TCR like fusion molecule comprises one or two immunoglobulin variable region(s). In certain embodiments, the extracellular antigen-binding domain of the TCR like fusion molecule comprises a heavy chain variable region (VH) of an antibody. In certain embodiments, the extracellular antigen-binding domain of the TCR like fusion molecule comprises a light chain variable region (VL) of an antibody. In certain embodiments, the extracellular antigen-binding domain of the TCR like fusion molecule is capable of dimerizing with another extracellular antigen-binding domain.

In certain embodiments, the extracellular antigen-binding domain of the TCR like fusion molecule comprises a VH of an antibody, wherein the VH is capable of dimerizing with another extracellular antigen-binding domain comprising a VL of the antibody and form a fragment variable (Fv). In certain embodiments, the extracellular antigen-binding domain of the TCR like fusion molecule comprises a VL of an antibody, wherein the VL is capable of dimerizing with another extracellular antigen-binding domain comprising a VH of the antibody and form a fragment variable (Fv).

In certain embodiments, the TCR like fusion molecule comprises an extracellular antigen-binding domain that specifically binds to HPV (e.g., an HPV/MHC complex). In certain embodiments, the extracellular antigen-binding domain of the TCR like fusion molecule comprises a V_{H} disclosed herein, e.g., any one of those disclosed in Section 5.3.1. In certain embodiments, the extracellular antigen-binding domain of the TCR like fusion molecule comprises a VL disclosed herein, e.g., any one of those disclosed in Section 5.3.1.

### 5.4.2. T Cell Receptor Fusion Construct (TRuC)

In certain embodiments, the antigen-recognizing receptor is a T cell receptor fusion construct (TRuC). Non-limiting examples of T cell receptor fusion constructs (TRuCs) are disclosed in Baeuerle et al., “Synthetic TRuC receptors engaging the complete T cell receptor for potent anti-tumor response,” Nature Communications volume 10, Article number: 2087 (2019), which is incorporated by reference in its entirety). A T cell receptor fusion construct is a recombinant polypeptide that is capable of binding to an antigen on a target cells and interacting with a member of the T cell receptor complex (e.g., TCRα, TCRβ, TCRγ, TCRδ, CD3γ, CD3δ, CD3ε, and CD3ζ).

In certain embodiments, the TruC comprises an extracellular antigen-binding domain and a member of the T cell receptor complex. In certain embodiments, the TruC comprises an extracellular antigen-binding domain that specifically binds to HPV (e.g., an HPV/MHC complex) and a member of the T cell receptor complex. The extracellular antigen-binding domain of the TRuC can include an scFv disclosed herein, e.g., any one of those disclosed in Section 5.3.1.

In addition, the extracellular antigen-binding domain can comprise a leader or a signal peptide that directs the nascent protein into the endoplasmic reticulum. Signal peptide or leader can be essential if the TRuC is to be glycosylated and anchored in the cell membrane. The signal sequence or leader can be a peptide sequence (about 5, about 10, about 15, about 20, about 25, or about 30 amino acids long) present at the N-terminus of newly synthesized proteins that directs their entry to the secretory pathway. In certain embodiments, the signal peptide is covalently joined to the N-terminus of the extracellular antigen-binding domain.

In certain embodiments, the extracellular antigen-binding domain of the TRuC is covalently joined to the N-terminus of the member of the T cell receptor complex. In certain embodiments, the member of the T cell receptor complex can be a native or a modified T cell receptor a chain polypeptide or a fragment thereof, a native or a modified T cell receptor R chain polypeptide or a fragment thereof, a native or a modified T cell receptor 7 chain polypeptide or a fragment thereof, a native or a modified T cell receptor 6 chain polypeptide or a fragment thereof, a native or a modified CD37 polypeptide or a fragment thereof, a native or a modified CD36 polypeptide or a fragment thereof, a native or a modified CD3F polypeptide or a fragment thereof, and a native or a modified CD3ζ polypeptide or a fragment thereof).

In certain embodiments, the extracellular antigen-binding domain and the member of the T cell receptor complex are linked via a linker. In certain embodiments, the linker comprises or consists of the amino acid sequence set forth in SEQ ID NO: 44, SEQ ID NO: 45, SEQ ID NO: 46, SEQ ID NO: 47, SEQ ID NO: 48, or SEQ ID NO: 49.

### 5.5. Cells Comprising the Antibodies or Antigen-Recognizing Receptors

The presently disclosed subject matter provides cells comprising a presently disclosed antigen-recognizing receptor (e.g., one disclosed in Section 5.4) or a presently disclosed antibody or antigen-binding fragment thereof (e.g., one disclosed in Section 5.3). In certain embodiments, the cell is selected from the group consisting of cells of lymphoid lineage, cells of myeloid lineage, stem cells from which cells of lymphoid lineage can be derived, and stem cells from which cells of myeloid lineage can be derived. In certain embodiments, the cell is an immunoresponsive cell. In certain embodiments, the immunoresponsive cell is a cell of lymphoid lineage.

In certain embodiments, the cell is a cell of the lymphoid lineage. Cells of the lymphoid lineage can provide production of antibodies, regulation of cellular immune system, detection of foreign agents in the blood, detection of cells foreign to the host, and the like. Non-limiting examples of cells of the lymphoid lineage include T cells, Natural Killer (NK) cells, B cells, dendritic cells, stem cells from which lymphoid cells may be differentiated. In certain embodiments, the stem cell is a pluripotent stem cell (e.g., embryonic stem cell or induced pluripotent stem cell (iPSC)).

In certain embodiments, the cell is a T cell. T cells can be lymphocytes that mature in the thymus and are chiefly responsible for cell-mediated immunity. T cells are involved in the adaptive immune system. The T cells of the presently disclosed subject matter can be any type of T cells, including, but not limited to, helper T cells, cytotoxic T cells, memory T cells (including central memory T cells, stem-cell-like memory T cells (or stem-like memory T cells), and two types of effector memory T cells: e.g., TEM cells and TEMRA cells, Regulatory T cells (also known as suppressor T cells), tumor-infiltrating lymphocyte (TIL), Natural killer T cells, Mucosal associated invariant T cells, and T6 T cells. Cytotoxic T cells (CTL or killer T cells) are a subset of T lymphocytes capable of inducing the death of infected somatic or tumor cells. A patient's own T cells may be genetically modified to target specific antigens through the introduction of an antigen-recognizing receptor, e.g., a CAR. In certain embodiments, the immunoresponsive cell is a T cell. The T cell can be a CD4⁺ T cell or a CD8⁺ T cell. In certain embodiments, the T cell is a CD4⁺ T cell. In certain embodiments, the T cell is a CD8⁺ T cell.

In certain embodiments, the cell is a NK cell. Natural killer (NK) cells can be lymphocytes that are part of cell-mediated immunity and act during the innate immune response. NK cells do not require prior activation in order to perform their cytotoxic effect on target cells.

Types of human lymphocytes of the presently disclosed subject matter include, without limitation, peripheral donor lymphocytes. e.g., those disclosed in Sadelain et al., Nat Rev Cancer (2003); 3:35-45 (disclosing peripheral donor lymphocytes genetically modified to express CARs), in Morgan, R. A., et al. 2006 Science 314:126-129 (disclosing peripheral donor lymphocytes genetically modified to express a full-length tumor antigen-recognizing T cell receptor complex comprising the a and R heterodimer), in Panelli et al., J Immunol (2000); 164:495-504; Panelli et al., J Immunol (2000); 164:4382-4392 (disclosing lymphocyte cultures derived from tumor infiltrating lymphocytes (TILs) in tumor biopsies), and in Dupont et al., Cancer Res (2005); 65:5417-5427; Papanicolaou et al., Blood (2003); 102:2498-2505 (disclosing selectively in vitro-expanded antigen-specific peripheral blood leukocytes employing artificial antigen-presenting cells (AAPCs) or pulsed dendritic cells).

The cells (e.g., T cells) can be autologous, non-autologous (e.g., allogeneic), or derived in vitro from engineered progenitor or stem cells.

The cells of the presently disclosed subject matter can be cells of the myeloid lineage. Non-limiting examples of cells of the myeloid lineage include monocytes, macrophages, neutrophils, dendritic cells, basophils, neutrophils, eosinophils, megakaryocytes, mast cell, erythrocyte, thrombocytes, and stem cells from which myeloid cells may be differentiated. In certain embodiments, the stem cell is a pluripotent stem cell (e.g., an embryonic stem cell or an induced pluripotent stem cell).

In certain embodiments, the presently disclosed cells are capable of modulating the tumor microenvironment. Tumors have a microenvironment that is hostile to the host immune response involving a series of mechanisms by malignant cells to protect themselves from immune recognition and elimination. This “hostile tumor microenvironment” comprises a variety of immune suppressive factors including infiltrating regulatory CD4⁺ T cells (Tregs), myeloid derived suppressor cells (MDSCs), tumor associated macrophages (TAMs), immune suppressive cytokines including TGF-β, and expression of ligands targeted to immune suppressive receptors expressed by activated T cells (CTLA-4 and PD-1). These mechanisms of immune suppression play a role in the maintenance of tolerance and suppressing inappropriate immune responses, however within the tumor microenvironment these mechanisms prevent an effective anti-tumor immune response.

In certain embodiments, the cells can be transduced with the presently disclosed HPV-targeted antigen-recognizing receptor such that the cells express the antigen-recognizing receptor.

### 5.6. Nucleic Acids Encoding the Antibodies or Antigen-Recognizing Receptors

The presently disclosed subject matter provides nucleic acids encoding the anti-HPV antibodies or antigen-binding fragments thereof disclosed herein. The presently disclosed subject matter also provides nucleic acids encoding the heavy chain variable region sequence of any one of the presently disclosed anti-HPV antibodies (e.g., 3F8, P1-13, P1-75, P1-80, P2-75-1, P2-75-5, P3-30, P4-10, R4-D1, 2A5, and 1B1 antibodies). In certain embodiments, the nucleic acid comprises or consists of a nucleotide sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the nucleotide sequence set forth in SEQ ID NO: 52, SEQ ID NO: 54, or SEQ ID NO: 56. In certain embodiments, the nucleic acid comprises or consists of the nucleotide sequence set forth in in SEQ ID NO: 52, SEQ ID NO: 54, or SEQ ID NO: 56.

The presently disclosed subject matter provides nucleic acids encoding the light chain variable region sequence of any one of the presently disclosed anti-HPV antibodies (e.g., 3F8, P1-13, P1-75, P1-80, P2-75-1, P2-75-5, P3-30, P4-10, R4-D1, 2A5, and 1B1 antibodies). In certain embodiments, the nucleic acid comprises or consists of a nucleotide sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the nucleotide sequence set forth in in SEQ ID NO: 53, SEQ ID NO: 55, or SEQ ID NO: 57. In certain embodiments, the nucleic acid comprises or consists of the nucleotide sequence set forth in SEQ ID NO: 53, SEQ ID NO: 55, or SEQ ID NO: 57.

Furthermore, the presently disclosed subject matter also provides nucleic acid molecules that encode the presently disclosed antigen-recognizing receptors (e.g., one disclosed in Section 5.4). In certain embodiments, the nucleic acid molecule comprises a nucleotide sequence that encodes a polypeptide of an HPV-targeted antigen recognizing receptor disclosed herein.

Further provided are vectors comprising the presently disclosed nucleic acids. In certain embodiments, the vector is an expression vector. The presently disclosed subject matter further provides host cells comprising the vectors disclosed herein. In certain embodiments, the host cells are T cells.

In certain non-limiting embodiments, the presently disclosed subject matter include use of colloids comprising nucleic acids molecules disclosed herein. As used herein, the term “colloid” refers to systems in which there are two or more phases, with one phase (e.g., the dispersed phase) distributed in the other phase (e.g., the continuous phase). Moreover, at least one of the phases has small dimensions (in the range of about 10⁻⁹ to about 10⁻⁶ m). Non-limiting examples of colloids encompassed by the presently disclosed subject matter include macromolecule complexes, nanocapsules, microspheres, beads, and lipid-based systems (e.g., micelles, liposomes, and lipid nanoparticles).

In certain non-limiting embodiments, the presently disclosed subject matter include use of liposomes comprising nucleic acids molecules disclosed herein. The term “liposome,” as used herein, refers to single- or multi-layered spherical lipid bilayer structures produced from lipids dissolved in organic solvents and then dispersed in aqueous media. Experimentally and therapeutically used for delivering an active pharmaceutical ingredient (e.g., nucleic acid compositions disclosed herein) to cells, liposomes fuse with cell membranes so the contents are transferred into the cytoplasm.

In certain non-limiting embodiments, the presently disclosed subject matter include use of lipid nanoparticles comprising nucleic acids molecules disclosed herein. As used herein, the term “lipid nanoparticle” refers to a particle having at least one dimension in the order of nanometers (e.g., from about 1 nm to about 1,000 nm) and including at least one lipid. In certain embodiments, the lipid nanoparticles can include an active pharmaceutical ingredient (e.g., nucleic acid compositions disclosed herein) for delivering to cells. The morphology of the lipid nanoparticles can be different from liposomes. While liposomes are characterized by a lipid bilayer surrounding a hydrophilic core, lipid nanoparticles have an electron-dense core where cationic lipids and/or ionizable lipids are organized into inverted micelles around an active pharmaceutical ingredient (e.g., nucleic acid compositions disclosed herein). Additional information on the morphology and properties of lipid nanoparticles and liposomes can be found in Wilczewska, et al., Pharmacological reports 64, no. 5 (2012): 1020-1037; Eygeris et al., Accounts of Chemical Research 55, no. 1 (2021): 2-12; Zhang et al., Chemical Reviews 121, no. 20 (2021): 12181-12277; and Fan et al., Journal of pharmaceutical and biomedical analysis 192 (2021): 113642.

In certain embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm.

In certain embodiments, the lipid nanoparticles can include a cationic lipid or an ionizable lipid. The term “cationic lipid” refers to lipids including a head group with permanent positive charges. Non-limiting examples of cationic lipids encompassed by the presently disclosed subject matter include 1,2-di-O-octadecenyl-3-trimethylammonium-propane (DOTMA), 1,2-dioleoyl-3-trimethylammonium-propane (DOTAP), 2,3-dioleyloxy-N-[2-(sperminecarboxamido)ethyl]-N,N-dimethyl-1-propanaminium trifluoroacetate (DOSPA), and ethylphosphatidylcholine (ePC).

As used herein, the term “ionizable lipid” refers to lipids that are protonated at low pH and are neutral at physiological pH. The pH-sensitivity of ionizable lipids is particularly beneficial for delivery in vivo (e.g., delivery of nucleic acid compositions disclosed herein), because neutral lipids have less interactions with the anionic membranes of blood cells and, thus, improve the biocompatibility of the lipid nanoparticles. Once trapped in endosomes, ionizable lipids are protonated and promote membrane destabilization to allow the endosomal escape of the nanoparticles. Non-limiting example of ionizable lipids encompassed by the presently disclosed subject matter include tetrakis(8-methylnonyl) 3,3′,3″,3′″-(((methylazanediyl) bis(propane-3,1 diyl))bis (azanetriyl))tetrapropionate; decyl (2-(dioctylammonio)ethyl) phosphate; ((4-hydroxybutyl)azanediyl)bis(hexane-6,1-diyl)bis(2-hexyldecanoate); bis(2-(dodecyldisulfanyl)ethyl) 3,3′-((3-methyl-9-oxo-10-oxa-13,14-dithia-3,6-diazahexacosyl)azanediyl)dipropionate; 1,1′-((2-(4-(2-((2-(bis(2-hydroxydodecyl)amino)ethyl) (2-hydroxydodecyl)amino)ethyl) piperazin-1-yl)ethyl)azanediyl) bis(dodecan-2-ol); cKK-E12, 3,6-bis(4-(bis(2-hydroxydodecyl)amino)butyl)piperazine-2,5-dione; (6Z,9Z,28Z,31Z)-heptatriaconta-6,9,28,31-tetraen-19-yl 4-(dimethylamino) butanoate; hexa(octan-3-yl) 9,9′,9″,9′″,9″″,9′″″-((((benzene-1,3,5-tricarbonyl)yris(azanediyl)) tris (propane-3,1-diyl)) tris(azanetriyl))hexanonanoate; heptadecan-9-yl 8-((2-hydroxyethyl)(6-oxo-6-(undecyloxy)hexyl)amino) octanoate; and (((3,6-dioxopiperazine-2,5-diyl)bis(butane-4, 1-diyl))bis(azanetriyl))tetrakis(ethane-2,1-diyl) (9Z,9′Z,9″Z,9′″Z,12Z,12′Z,12″Z,12′″Z)-tetrakis (octadeca-9,12-dienoate).

Additionally, in certain embodiments, the lipid nanoparticles can include other lipids. For example, but without any limitation, the lipid nanoparticles of the presently disclosed subject matter can include phospholipids, cholesterol, polyethylene glycol (PEG)-functionalized lipids (PEG-lipids). These lipids can improve certain properties of the lipid nanoparticles (e.g., stability, biodistribution, etc.). For example, cholesterol enhances the stability of the lipid nanoparticles by modulating the integrity and rigidity. Non-limiting examples of other lipids present in lipid nanoparticles include cholesterol, DC-cholesterol, β-sitosterol, BHEM-cholesterol, ALC-0159, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoylphosphatidylethanolamine (DSPE), 16-O-monomethyl PE, 16-O-dimethyl PE, 18-1-trans PE, 1-stearioyl-2-oleoyl-phosphatidyethanol amine (SOPE), and 1,2-dielaidoyl-sn-glycero-3-phophoethanolamine (transDOPE).

In certain embodiments, the lipid nanoparticles can include a targeting moiety that binds to a ligand. The use of the targeting moieties allows selective delivery of an active pharmaceutical ingredient (e.g., nucleic acid compositions disclosed herein) to target cells expressing the ligand (e.g., T cells). In certain embodiments, the targeting moiety can be an antibody or antigen-binding fragment thereof that binds to a cell surface receptor. For example, but without any limitation, the targeting domain is an antibody or antigen-binding fragment thereof that binds to a receptor expressed on the surface of a T cell (e.g., CD3, CD4, CD8, CD16, CD40L, CD95, FasL, CTLA-4, OX40, GITR, LAG3, ICOS, and PD-1).

### 5.7. Pharmaceutical Compositions and Methods of Treatment

The presently disclosed subject matter provides compositions comprising a presently disclosed anti-HPV antibody or an antigen-binding fragment thereof, a presently disclosed immunoconjugate, a presently disclosed multi-specific molecule, or a presently disclosed antigen-recognizing receptor, or a presently disclosed cell. In certain embodiments, the composition is a pharmaceutical composition further comprising a pharmaceutically acceptable carrier.

Suitable pharmaceutically acceptable carriers include, for example, one or more of water, saline, phosphate buffered saline, dextrose, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the binding proteins. The compositions of the injection can, as is well known in the art, be formulated so as to provide quick, sustained or delayed release of the active ingredient after administration to the mammal.

The presently disclosed subject matter provides various methods of using the anti-HPV antibodies or antigen-binding fragments thereof, the immunoconjugate, the multi-specific molecule, the antigen-recognizing receptor, the cell, and the composition disclosed herein. For example, the presently disclosed subject matter provides methods for treating or ameliorating a disease or disorder in a subject. In certain embodiments, the method comprises administering to the subject one or more of the anti-HPV antibodies or antigen-binding fragments thereof, the immunoconjugate, the multi-specific molecule, the antigen-recognizing receptor, the cell, or the composition disclosed herein. In certain embodiments, the disease or disorder is associated with HPV. In certain embodiments, the disease or disorder is tumor.

The presently disclosed subject matter provides methods of reducing tumor burden in a subject. In certain embodiments, the method comprises administering to the subject one or more of the anti-HPV antibodies or antigen-binding fragments thereof, the immunoconjugate, the multi-specific molecule, the antigen-recognizing receptor, the cell, or the composition disclosed herein. The presently disclosed anti-HPV antibodies or antigen-binding fragments thereof can reduce the number of tumor cells, reduce tumor size, and/or eradicate the tumor in the subject.

The presently disclosed subject matter also provides methods of increasing or lengthening survival of a subject having a tumor. In certain embodiments, the method comprises administering to the subject one or more of the anti-HPV antibodies or antigen-binding fragments thereof, the immunoconjugate, the multi-specific molecule, the antigen-recognizing receptor, the cell, or the composition disclosed herein. The method can reduce or eradicate tumor burden in the subject.

The presently disclosed subject matter further provides methods for treating and/or preventing a tumor in a subject. In certain embodiments, the method comprises administering to the subject one or more of the anti-HPV antibodies or antigen-binding fragments thereof, the immunoconjugate, the multi-specific molecule, the antigen-recognizing receptor, the cell, or the composition disclosed herein.

Such methods comprise administering the presently disclosed anti-HPV antibodies or antigen-binding fragments thereof or the cells in an amount effective, a presently disclosed composition (e.g., a pharmaceutical composition) to achieve the desired effect, be it palliation of an existing condition or prevention of recurrence. For treatment, the amount administered is an amount effective in producing the desired effect. An effective amount can be provided in one or a series of administrations. An effective amount can be provided in a bolus or by continuous perfusion.

In certain embodiments, the tumor is cancer. In certain embodiments, the tumor is an HPV-associated tumor. Non-limiting examples of HPV-associated tumors include ovarian cancer, cervical cancer, anal cancer, vaginal cancer, vulvar cancer, penile cancer, head and neck cancer, oropharynx cancer, oropharyngeal squamous cell carcinoma (SCC), anal & rectal SCC, vulvar SCC, vaginal SCC, cervical carcinoma, and penile SCC. In certain embodiments, the tumor is ovarian cancer.

Any suitable method or route can be used to administer a presently disclosed anti-HPV antibody, and optionally, to co-administer antineoplastic agents. Routes of administration include, but are not limited to, oral, intravenous, intraperitoneal, subcutaneous, intramuscular, intranodal, intratumoral, intraosseous, intrathecal, pleural, intrapleural, topical, and direct administration. It should be emphasized, however, that the presently disclosed subject matter is not limited to any particular method or route of administration.

The presently disclosed anti-HPV antibodies or antigen-binding fragments thereof can be administered as a conjugate, which binds specifically to the receptor and delivers a toxic, lethal payload following ligand-toxin internalization.

### 5.8. Diagnostic and Prognostic Methods

The presently disclosed anti-HPV antibodies, antigen-binding fragments thereof, multi-specific molecules, and nucleic acids encode thereof can be used for diagnostic and prognostic applications as well as use as research tools for detection of HPV (e.g., an HPV/HLA complex, more specifically, a HPV/HLA class I complex, more specifically, a HPV/HLA-A complex, more specifically, a HPV/HLA-A2 complex, or more specifically, a HPV/HLA-A*02:01 complex) in a biological sample, in a cell, a tissue, or a blood sample. The presently disclosed subject matter provides methods for detecting HPV (e.g., an HPV/HLA complex, more specifically, a HPV/HLA class I complex, more specifically, a HPV/HLA-A complex, more specifically, a HPV/HLA-A2 complex, or more specifically, a HPV/HLA-A*02:01 complex) in a cell, a tissue, or a blood sample. In certain embodiments, the method comprises: contacting a cell, a tissue, or a blood sample with the antibody, antigen-binding fragment thereof, or multi-specific molecule disclosed herein, wherein the antibody, antigen-binding fragment thereof or multi-specific molecule comprises a detectable label; and determining the amount of the labeled antibody, antigen-binding fragment thereof, or multi-specific molecule bound to the cell, tissue, or blood sample by measuring the amount of detectable label associated with the cell or tissue, wherein the amount of bound antibody, antigen-binding fragment thereof, or multi-specific molecule indicates the amount of HPV (e.g., an HPV/HLA complex, more specifically, a HPV/HLA class I complex, more specifically, a HPV/HLA-A complex, more specifically, a HPV/HLA-A2 complex, or more specifically, a HPV/HLA-A*02:01 complex) in the cell, tissue, or a blood sample. The cell or tissue can be any cell or tissue, including any normal, healthy, or cancerous cells and tissues. In certain embodiments, the blood sample is a peripheral blood sample.

The presently disclosed anti-HPV antibodies or antigen-binding fragments thereof can be used in methods known in the art relating to the localization and/or quantitation of HPV polypeptides (e.g., for use in measuring levels of the HPV protein within appropriate physiological samples, for use in diagnostic methods, for use in imaging the polypeptide, and the like). The presently disclosed anti-HPV antibodies or antigen-binding fragments thereof can be used to isolate an HPV/MHC complex by standard techniques, such as affinity chromatography or immunoprecipitation. The presently disclosed anti-HPV antibodies or antigen-binding fragments thereof can facilitate the purification of immunoreactive HPV cells from biological samples, e.g., mammalian cells expressing an HPV/MHC complex. The presently disclosed anti-HPV antibodies or antigen-binding fragments thereof can be used diagnostically to monitor immunoreactive HPV protein levels (e.g., an HPV/HLA complex, more specifically, a HPV/HLA class I complex, more specifically, a HPV/HLA-A complex, more specifically, a HPV/HLA-A2 complex, or more specifically, a HPV/HLA-A*02:01 complex) in tissue as part of a clinical testing procedure, e.g., to determine the efficacy of a given treatment regimen. As noted above, the detection can be facilitated by coupling (i.e., physically linking) the presently disclosed anti-HPV antibodies or antigen-binding fragments thereof to a detectable substance.

An exemplary method for detecting the presence or absence of an immunoreactive HPV protein in a biological sample comprises contacting a biological sample from a subject with a presently disclosed anti-HPV antibody or an antigen-binding fragment thereof, wherein the presence of an immunoreactive HPV protein (e.g., an HPV/HLA complex, more specifically, a HPV/HLA class I complex, more specifically, a HPV/HLA-A complex, more specifically, a HPV/HLA-A2 complex, or more specifically, a HPV/HLA-A*02:01 complex) is detected in the biological sample. Detection may be accomplished by means of a detectable label attached to the antibody.

The term “labeled” with regard to the anti-HPV antibody or antigen-binding fragment thereof is intended to encompass direct labeling of the antibody by coupling (i.e., physically linking) a detectable substance to the antibody, as well as indirect labeling of the antibody by reactivity with another compound that is directly labeled, such as a secondary antibody. Examples of indirect labeling include detection of a primary antibody using a fluorescently-labeled secondary antibody and end-labeling of a DNA probe with biotin such that it can be detected with fluorescently-labeled streptavidin.

In certain embodiments, the presently disclosed anti-HPV antibodies or antigen-binding fragments thereof are conjugated to one or more detectable labels. For such uses, the presently disclosed anti-HPV antibodies or antigen-binding fragments thereof may be detectably labeled by covalent or non-covalent attachment of a chromogenic, enzymatic, radioisotopic, isotopic, fluorescent, toxic, chemiluminescent, nuclear magnetic resonance contrast agent or other label.

The presently disclosed detection methods can be used to detect an immunoreactive HPV protein in a biological sample in vitro as well as in vivo. Non-limiting examples of in vitro techniques for detection of an immunoreactive HPV protein (e.g., an HPV/HLA complex, more specifically, a HPV/HLA class I complex, more specifically, a HPV/HLA-A complex, more specifically, a HPV/HLA-A2 complex, or more specifically, a HPV/HLA-A*02:01 complex) include enzyme linked immunosorbent assays (ELISAs), Western blots, immunoprecipitations, radioimmunoassay, and immunofluorescence. Furthermore, in vivo techniques for detection of an immunoreactive HPV protein include introducing into a subject a labeled anti-HPV antibody or an antigen-binding fragment thereof. For example, the anti-HPV antibody or antigen-binding fragment thereof can be labeled with a radioactive marker whose presence and location in a subject can be detected by standard imaging techniques. In certain embodiments, the biological sample comprises cells expressing HPV/MHC complexes from the test subject.

The presently disclosed anti-HPV antibodies or antigen-binding fragments thereof can be used to assay immunoreactive HPV protein levels in a biological sample (e.g., human plasma) using antibody-based techniques. For example, protein expression in tissues can be studied with classical immunohistological methods. Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase, and radioisotopes or other radioactive agent, such as iodine (¹²⁵I, ¹²¹I, ¹³¹I), carbon (¹⁴C), sulfur (³⁵S), tritium (3H), indium (¹¹¹In), and technetium (^{99m}Tc), and fluorescent labels, such as fluorescein, rhodamine, and green fluorescent protein (GFP), as well as biotin.

In addition to assaying immunoreactive HPV protein levels in a biological sample, the presently disclosed anti-HPV antibodies or antigen-binding fragments thereof may be used for in vivo imaging of HPV. Antibodies useful for this method include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which can be incorporated into the anti-HPV antibodies by labeling of nutrients for the relevant scFv clone.

The presently disclosed anti-HPV antibodies or antigen-binding fragments thereof, which are labeled with an appropriate detectable imaging moiety (such as a radioisotope (e.g., ¹³¹I, ¹¹¹IN ^{99m}Tc, ¹⁸F, ⁸⁹Zr), a radio-opaque substance, or a material detectable by nuclear magnetic resonance) are introduced (e.g., parenterally, subcutaneously, or intraperitoneally) into the subject. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ^{99m}Tc. The labeled anti-HPV antibody or antigen-binding fragment thereof then accumulates at the location of cells which contain the specific target polypeptide. For example, the labeled anti-HPV antibodies or antigen-binding fragments thereof accumulate within the subject in cells and tissues in which the HPV protein has localized.

Thus, the presently disclosed subject matter provides diagnostic methods of a medical condition. In certain embodiments, the method comprises: (a) assaying the expression of immunoreactive HPV protein by measuring binding of a presently disclosed anti-HPV antibody or an antigen-binding fragment thereof in cells or body fluid of an individual; and (b) comparing the amount of immunoreactive HPV protein present in the sample with a standard reference, wherein an increase or decrease in immunoreactive HPV protein levels compared to the standard is indicative of a medical condition.

Furthermore, the presently disclosed anti-HPV antibodies or antigen-binding fragments thereof may be used to purify immunoreactive HPV protein from a sample. In certain embodiments, the antibodies are immobilized on a solid support. Non-limiting examples of such solid supports include plastics such as polycarbonate, complex carbohydrates such as agarose and sepharose, acrylic resins and such as polyacrylamide and latex beads. Techniques for coupling antibodies to such solid supports are well known in the art.

The simplest method to bind the antigen to the antibody-support matrix is to collect the beads in a column and pass the antigen solution down the column. The efficiency of this method depends on the contact time between the immobilized antibody and the antigen, which can be extended by using low flow rates. The immobilized antibody captures the antigen as it flows past. Alternatively, an antigen can be contacted with the antibody-support matrix by mixing the antigen solution with the support (e.g., beads) and rotating the slurry, allowing maximum contact between the antigen and the immobilized antibody. After the binding reaction has been completed, the slurry is passed into a column for collection of the beads. The beads are washed using a suitable washing buffer and then the pure or substantially pure antigen is eluted.

An antibody or polypeptide of interest can be conjugated to a solid support, such as a bead. In addition, a first solid support such as a bead can also be conjugated, if desired, to a second solid support, which can be a second bead or other support, by any suitable means, including those disclosed herein for conjugation of a polypeptide to a support. Accordingly, any of the conjugation methods and means disclosed herein with reference to conjugation of a polypeptide to a solid support can also be applied for conjugation of a first support to a second support, where the first and second solid support can be the same or different.

Appropriate linkers, which can be cross-linking agents, for use for conjugating a polypeptide to a solid support include a variety of agents that can react with a functional group present on a surface of the support, or with the polypeptide, or both. Reagents useful as cross-linking agents include homo-bi-functional and, in particular, hetero-bi-functional reagents. Useful bi-functional cross-linking agents include, but are not limited to, N-SIAB, dimaleimide, DTNB, N-SATA, N-SPDP, SMCC and 6-HYNIC. A cross-linking agent can be selected to provide a selectively cleavable bond between a polypeptide and the solid support. For example, a photolabile cross-linker, such as 3-amino-(2-nitrophenyl)propionic acid can be employed as a means for cleaving a polypeptide from a solid support. (Brown et al., Mol. Divers, pp, 4-12 (1995); Rothschild et al., Nucl. Acids Res., 24:351-66 (1996); and U.S. Pat. No. 5,643,722). Other cross-linking reagents are well-known in the art. (See, e.g., Wong (1991), supra; and Hermanson (1996), supra).

An antibody or polypeptide can be immobilized on a solid support, such as a bead, through a covalent amide bond formed between a carboxyl group functionalized bead and the amino terminus of the polypeptide or, conversely, through a covalent amide bond formed between an amino group functionalized bead and the carboxyl terminus of the polypeptide. In addition, a bi-functional trityl linker can be attached to the support, e.g., to the 4-nitrophenyl active ester on a resin, such as a Wang resin, through an amino group or a carboxyl group on the resin via an amino resin. Using a bi-functional trityl approach, the solid support can require treatment with a volatile acid, such as formic acid or trifluoroacetic acid to ensure that the polypeptide is cleaved and can be removed. In such a case, the polypeptide can be deposited as a beadless patch at the bottom of a well of a solid support or on the flat surface of a solid support. After addition of a matrix solution, the polypeptide can be desorbed into a MS.

Hydrophobic trityl linkers can also be exploited as acid-labile linkers by using a volatile acid or an appropriate matrix solution, e.g., a matrix solution containing 3-HPA, to cleave an amino linked trityl group from the polypeptide. Acid lability can also be changed. For example, trityl, monomethoxytrityl, dimethoxytrityl or trimethoxytrityl can be changed to the appropriate p-substituted, or more acid-labile tritylamine derivatives, of the polypeptide, i.e., trityl ether and tritylamine bonds can be made to the polypeptide. Accordingly, a polypeptide can be removed from a hydrophobic linker, e.g., by disrupting the hydrophobic attraction or by cleaving tritylether or tritylamine bonds under acidic conditions, including, if desired, under typical MS conditions, where a matrix, such as 3-HPA acts as an acid.

Orthogonally cleavable linkers can also be useful for binding a first solid support, e.g., a bead to a second solid support, or for binding a polypeptide of interest to a solid support. Using such linkers, a first solid support, e.g., a bead, can be selectively cleaved from a second solid support, without cleaving the polypeptide from the support; the polypeptide then can be cleaved from the bead at a later time. For example, a disulfide linker, which can be cleaved using a reducing agent, such as DTT, can be employed to bind a bead to a second solid support, and an acid cleavable bi-functional trityl group could be used to immobilize a polypeptide to the support. As desired, the linkage of the polypeptide to the solid support can be cleaved first, e.g., leaving the linkage between the first and second support intact. Trityl linkers can provide a covalent or hydrophobic conjugation and, regardless of the nature of the conjugation, the trityl group is readily cleaved in acidic conditions.

For example, a bead can be bound to a second support through a linking group which can be selected to have a length and a chemical nature such that high density binding of the beads to the solid support, or high density binding of the polypeptides to the beads, is promoted. Such a linking group can have, e.g., “tree-like” structure, thereby providing a multiplicity of functional groups per attachment site on a solid support. Examples of such linking group; include polylysine, polyglutamic acid, penta-erythrole and tris-hydroxy-aminomethane.

Noncovalent Binding Association. An antibody or polypeptide can be conjugated to a solid support, or a first solid support can also be conjugated to a second solid support, through a noncovalent interaction. For example, a magnetic bead made of a ferromagnetic material, which is capable of being magnetized, can be attracted to a magnetic solid support, and can be released from the support by removal of the magnetic field. Alternatively, the solid support can be provided with an ionic or hydrophobic moiety, which can allow the interaction of an ionic or hydrophobic moiety, respectively, with a polypeptide, e.g., a polypeptide containing an attached trityl group or with a second solid support having hydrophobic character.

A solid support can also be provided with a member of a specific binding pair and, therefore, can be conjugated to a polypeptide or a second solid support containing a complementary binding moiety. For example, a bead coated with avidin or with streptavidin can be bound to a polypeptide having a biotin moiety incorporated therein, or to a second solid support coated with biotin or derivative of biotin, such as iminobiotin.

It should be recognized that any of the binding members disclosed herein or otherwise known in the art can be reversed. Thus, biotin, e.g., can be incorporated into either a polypeptide or a solid support and, conversely, avidin or other biotin binding moiety would be incorporated into the support or the polypeptide, respectively. Other specific binding pairs contemplated for use herein include, but are not limited to, hormones and their receptors, enzyme, and their substrates, a nucleotide sequence and its complementary sequence, an antibody and the antigen to which it interacts specifically, and other such pairs knows to those skilled in the art.

The presently disclosed anti-HPV antibodies or antigen-binding fragments thereof are useful in diagnostic methods. As such, the presently disclosed subject matter provides methods using the presently disclosed anti-HPV antibodies or antigen-binding fragments thereof in diagnosis of HPV activity in a subject. The presently disclosed anti-HPV antibodies or antigen-binding fragments thereof may be selected such that they have any level of epitope binding specificity and high binding affinity to a HPV polypeptide.

The presently disclosed anti-HPV antibodies or antigen-binding fragments thereof can be used to detect an immunoreactive HPV protein in a variety of standard assay formats. Such formats include immunoprecipitation, Western blotting, ELISA, radioimmunoassay, and immunometric assays. Biological samples can be obtained from any tissue or body fluid of a subject. In certain embodiments, the subject is at an early stage of cancer. In certain embodiments, the early stage of cancer is determined by the level or expression pattern of HPV protein in a sample obtained from the subject. In certain embodiments, the sample is selected from the group consisting of urine, blood, serum, plasma, saliva, amniotic fluid, cerebrospinal fluid (CSF), and biopsied body tissue.

Immunometric or sandwich assays are one format for the diagnostic methods of the present technology. Such assays use one antibody, e.g., the anti-HPV antibody or a population of anti-HPV antibodies immobilized to a solid phase, and another anti-HPV antibody or a population of anti-HPV antibodies in solution. Typically, the solution anti-HPV antibody or population of anti-HPV antibodies is labeled. If an antibody population is used, the population can contain antibodies binding to different epitope specificities within the target polypeptide. Accordingly, the same population can be used for both solid phase and solution antibody. If anti-HPV monoclonal antibodies are used, first and second HPV monoclonal antibodies having different binding specificities are used for the solid and solution phase. Solid phase (also referred to as “capture”) and solution (also referred to as “detection”) antibodies can be contacted with target antigen in either order or simultaneously. If the solid phase antibody is contacted first, the assay is referred to as being a forward assay. Conversely, if the solution antibody is contacted first, the assay is referred to as being a reverse assay. If the target is contacted with both antibodies simultaneously, the assay is referred to as a simultaneous assay. After contacting the HPV protein with the anti-HPV antibody, a sample is incubated for a period that usually varies from about 10 min to about 24 hr and is usually about 1 hr. A wash step is then performed to remove components of the sample not specifically bound to the anti-HPV antibody being used as a diagnostic reagent. When solid phase and solution antibodies are bound in separate steps, a wash can be performed after either or both binding steps. After washing, binding is quantified, typically by detecting a label linked to the solid phase through binding of labeled solution antibody. Usually for a given pair of antibodies or populations of antibodies and given reaction conditions, a calibration curve is prepared from samples containing known concentrations of target antigen. Concentrations of the immunoreactive HPV protein in samples being tested are then read by interpolation from the calibration curve (i.e., standard curve). Analyte can be measured either from the amount of labeled solution antibody bound at equilibrium or by kinetic measurements of bound labeled solution antibody at a series of time points before equilibrium is reached. The slope of such a curve is a measure of the concentration of the HPV protein in a sample.

Suitable supports for use in the above methods include, e.g., nitrocellulose membranes, nylon membranes, and derivatized nylon membranes, and also particles, such as agarose, a dextran-based gel, dipsticks, particulates, microspheres, magnetic particles, test tubes, microtiter wells, SEPHADEX™ (Amersham Pharmacia Biotech, Piscataway N.J.), and the like. Immobilization can be by absorption or by covalent attachment. Optionally, anti-HPV antibodies can be joined to a linker molecule, such as biotin for attachment to a surface bound linker, such as avidin.

In certain embodiments, the presently disclosed anti-HPV antibody or antigen-binding fragment thereof is conjugated to a diagnostic agent. The diagnostic agent may comprise a radioactive or non-radioactive label, a contrast agent (such as for magnetic resonance imaging, computed tomography or ultrasound), and the radioactive label can be a gamma-, beta-, alpha-, Auger electron-, or positron-emitting isotope. A diagnostic agent is a molecule which is administered conjugated to an antibody moiety, i.e., antibody or antibody fragment, or subfragment, and is useful in diagnosing or detecting a disease by locating the cells comprising the antigen.

Useful diagnostic agents include, but are not limited to, radioisotopes, dyes (such as with the biotin-streptavidin complex), contrast agents, fluorescent compounds or molecules and enhancing agents (e.g., paramagnetic ions) for magnetic resonance imaging (MRI). In certain embodiments, the diagnostic agents are selected from the group consisting of radioisotopes, enhancing agents for use in magnetic resonance imaging, and fluorescent compounds. Chelates may be coupled to the presently disclosed anti-HPV antibodies or antigen-binding fragments thereof using standard chemistries. The chelate is normally linked to the antibody by a group which enables formation of a bond to the molecule with minimal loss of immunoreactivity and minimal aggregation and/or internal cross-linking.

### 5.9. Kits

The presently disclosed subject matter provides kits for treatment or ameliorating a disease or disorder associated with HPV (e.g., ovarian cancer), and/or detecting HPV/MHC complexes. In certain embodiments, the kit comprises the anti-HPV antibodies or antigen-binding fragments thereof, the immunoconjugate, the multi-specific molecule, the antigen-recognizing receptor, the cell, or the composition disclosed herein. In certain embodiments, the kit comprises a sterile container which contains a therapeutic or prophylactic vaccine; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

In certain embodiments, the kit further comprises instructions for administering the anti-HPV antibodies or antigen-binding fragments thereof, the immunoconjugate, the multi-specific molecule, the antigen-recognizing receptor, the cell, or the composition disclosed herein to a subject in need the treatment. The instructions can generally include information about the use of the anti-HPV antibodies or antigen-binding fragments thereof, the immunoconjugate, the multi-specific molecule, the antigen-recognizing receptor, the cell, and the composition disclosed herein for the treatment or ameliorating a disease or disorder. In certain embodiments, the instructions include at least one of the following: description of the therapeutic agent; dosage schedule and administration for treatment and/or prevention of a tumor or neoplasm or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

### 5.10. Exemplary Embodiments

A1. In certain non-limiting embodiments, the presently disclosed subject matter provides an antibody or an antigen-binding fragment thereof that binds to an HPV epitope bound to a human major histocompatibility complex (MHC) molecule, wherein the antibody or antigen-binding fragment thereof comprises a heavy chain variable region and a light chain variable region.
A2. The foregoing antibody or antigen-binding portion thereof of A1, wherein the MHC molecule is a human leukocyte antigen (HLA) molecule.
A3. The foregoing antibody or antigen-binding portion thereof of A2, wherein the HLA molecule is an HLA class I molecule.
A4. The foregoing antibody or antigen-binding portion thereof of A3, wherein the HLA class I molecule is HLA-A.
A5. The foregoing antibody or antigen-binding portion thereof of A4, wherein the HLA-A is HLA-A2.
A6. The foregoing antibody or antigen-binding portion thereof of A5, wherein the HLA-A2 is HLA-A*02:01.
A7. The foregoing antibody or antigen-binding portion thereof of any one of A1-A6, wherein the HPV epitope is an E7 epitope.
A8. The foregoing antibody or antigen-binding portion thereof of A7, wherein the E7 epitope comprises or consists of the amino acid sequence set forth in SEQ ID NO: 1.
A9. The foregoing antibody or antigen-binding fragment thereof of any one of A1-A8, wherein the heavy chain variable region comprises an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 9, SEQ ID NO: 34, or SEQ ID NO: 42.
A10. The foregoing antibody or antigen-binding fragment thereof of any one of A1-A8, wherein the light chain variable region comprises an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 10, SEQ ID NO: 12, SEQ ID NO: 15, SEQ ID NO: 17, SEQ ID NO: 20, SEQ ID NO: 22, SEQ ID NO: 24, SEQ ID NO: 26, SEQ ID NO: 28, SEQ ID NO: 35, or SEQ ID NO: 43.
A11. The foregoing antibody or antigen-binding fragment thereof of any one of A1-A8, wherein
(a) the heavy chain variable region comprises an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 9, SEQ ID NO: 34, or SEQ ID NO: 42; and
(b) the light chain variable region comprises an amino acid sequence that is at least about 80%, at least about 85%, at least about 90%, at least about 95%, at least about 96%, at least about 97%, at least about 98%, at least about 99% at least about 100% homologous or identical to the amino acid sequence set forth in SEQ ID NO: 10, SEQ ID NO: 12, SEQ ID NO: 15, SEQ ID NO: 17, SEQ ID NO: 20, SEQ ID NO: 22, SEQ ID NO: 24, SEQ ID NO: 26, SEQ ID NO: 28, SEQ ID NO: 35, or SEQ ID NO: 43.
A12. The foregoing antibody or antigen-binding fragment thereof of any one of A1-A8, wherein the heavy chain variable region and the light chain variable region are selected from the group consisting of:
(a) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 9, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 10;
(b) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 9, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 12;
(c) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 9, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 15;
(d) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 9, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 17;
(e) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 9, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 20;
(f) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 9, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 22;
(g) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 9, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 24;
(h) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 9, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 26;
(i) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 9, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 28;
(j) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 34, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 35; and
(k) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 42, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 43.
A13. The foregoing antibody or antigen-binding fragment thereof of any one of A1-A8, wherein the heavy chain variable region comprises the amino acid sequence set forth in SEQ ID NO: 9, SEQ ID NO: 34, or SEQ ID NO: 42.
A14. The foregoing antibody or antigen-binding fragment thereof of any one of A1-A8, wherein the light chain variable region comprises the amino acid sequence set forth in SEQ ID NO: 10, SEQ ID NO: 12, SEQ ID NO: 15, SEQ ID NO: 17, SEQ ID NO: 20, SEQ ID NO: 22, SEQ ID NO: 24, SEQ ID NO: 26, SEQ ID NO: 28, SEQ ID NO: 35, or SEQ ID NO: 43.
A15. The foregoing antibody or antigen-binding fragment thereof of any one of A1-A8, wherein
(a) the heavy chain variable region comprises the amino acid sequence set forth in SEQ ID NO: 9, SEQ ID NO: 34, or SEQ ID NO: 42; and
(b) the light chain variable region comprises the amino acid sequence set forth in SEQ ID NO: 10, SEQ ID NO: 12, SEQ ID NO: 15, SEQ ID NO: 17, SEQ ID NO: 20, SEQ ID NO: 22, SEQ ID NO: 24, SEQ ID NO: 26, SEQ ID NO: 28, SEQ ID NO: 35, or SEQ ID NO: 43.
A16. The foregoing antibody or antigen-binding fragment thereof of any one of A9-A15, wherein
(a) the heavy chain variable region comprises the amino acid sequence set forth in SEQ ID NO: 9, and the light chain variable region comprises the amino acid sequence set forth in SEQ ID NO: 10;
(b) the heavy chain variable region comprises the amino acid sequence set forth in SEQ ID NO: 9, and the light chain variable region comprises the amino acid sequence set forth in SEQ ID NO: 12;
(c) the heavy chain variable region comprises the amino acid sequence set forth in SEQ ID NO: 9, and the light chain variable region comprises the amino acid sequence set forth in SEQ ID NO: 15;
(d) the heavy chain variable region comprises the amino acid sequence set forth in SEQ ID NO: 9, and the light chain variable region comprises the amino acid sequence set forth in SEQ ID NO: 17;
(e) the heavy chain variable region comprises the amino acid sequence set forth in SEQ ID NO: 9, and the light chain variable region comprises the amino acid sequence set forth in SEQ ID NO: 20;
(f) the heavy chain variable region comprises the amino acid sequence set forth in SEQ ID NO: 9, and the light chain variable region comprises the amino acid sequence set forth in SEQ ID NO: 22;
(g) the heavy chain variable region comprises the amino acid sequence set forth in SEQ ID NO: 9, and the light chain variable region comprises the amino acid sequence set forth in SEQ ID NO: 24;
(h) the heavy chain variable region comprises the amino acid sequence set forth in SEQ ID NO: 9, and the light chain variable region comprises the amino acid sequence set forth in SEQ ID NO: 26;
(i) the heavy chain variable region comprises the amino acid sequence set forth in SEQ ID NO: 9, and the light chain variable region comprises the amino acid sequence set forth in SEQ ID NO: 28;
(j) the heavy chain variable region comprises the amino acid sequence set forth in SEQ ID NO: 34, and the light chain variable region comprises the amino acid sequence set forth in SEQ ID NO: 35; or
(k) the heavy chain variable region comprises the amino acid sequence set forth in SEQ ID NO: 42, and the light chain variable region comprises the amino acid sequence set forth in SEQ ID NO: 43.
A17. The foregoing antibody or antigen-binding fragment thereof of A16, wherein the heavy chain variable region comprises the amino acid sequence set forth in SEQ ID NO: 9, and the light chain variable region comprises the amino acid sequence set forth in SEQ ID NO: 10.
A18. The foregoing antibody or antigen-binding fragment thereof of any one of A1-A8, wherein the heavy chain variable region comprises CDR1, CDR2, and CDR3 domains; and the light chain variable region comprises CDR1, CDR2, and CDR3 domains, wherein the heavy chain variable region and light chain variable region CDR3 domains are selected from the group consisting of:
(a) a heavy chain variable region CDR3 comprising the amino acid sequence set forth in SEQ ID NO: 5 or a conservative modification thereof; and a light chain variable region CDR3 comprising the amino acid sequence set forth in SEQ ID NO: 8 or a conservative modification thereof;
(b) a heavy chain variable region CDR3 comprising the amino acid sequence set forth in SEQ ID NO: 5 or a conservative modification thereof; and a light chain variable region CDR3 comprising the amino acid sequence set forth in SEQ ID NO: 11 or a conservative modification thereof;
(c) a heavy chain variable region CDR3 comprising the amino acid sequence set forth in SEQ ID NO: 5 or a conservative modification thereof; and a light chain variable region CDR3 comprising the amino acid sequence set forth in SEQ ID NO: 14 or a conservative modification thereof;
(d) a heavy chain variable region CDR3 comprising the amino acid sequence set forth in SEQ ID NO: 5 or a conservative modification thereof; and a light chain variable region CDR3 comprising the amino acid sequence set forth in SEQ ID NO: 16 or a conservative modification thereof;
(e) a heavy chain variable region CDR3 comprising the amino acid sequence set forth in SEQ ID NO: 5 or a conservative modification thereof; and a light chain variable region CDR3 comprising the amino acid sequence set forth in SEQ ID NO: 19 or a conservative modification thereof;
(f) a heavy chain variable region CDR3 comprising the amino acid sequence set forth in SEQ ID NO: 5 or a conservative modification thereof; and a light chain variable region CDR3 comprising the amino acid sequence set forth in SEQ ID NO: 21 or a conservative modification thereof;
(g) a heavy chain variable region CDR3 comprising the amino acid sequence set forth in SEQ ID NO: 5 or a conservative modification thereof; and a light chain variable region CDR3 comprising the amino acid sequence set forth in SEQ ID NO: 23 or a conservative modification thereof;
(h) a heavy chain variable region CDR3 comprising the amino acid sequence set forth in SEQ ID NO: 5 or a conservative modification thereof; and a light chain variable region CDR3 comprising the amino acid sequence set forth in SEQ ID NO: 25 or a conservative modification thereof;
(i) a heavy chain variable region CDR3 comprising the amino acid sequence set forth in SEQ ID NO: 5 or a conservative modification thereof; and a light chain variable region CDR3 comprising the amino acid sequence set forth in SEQ ID NO: 27 or a conservative modification thereof;
(j) a heavy chain variable region CDR3 comprising the amino acid sequence set forth in SEQ ID NO: 30 or a conservative modification thereof; and a light chain variable region CDR3 comprising the amino acid sequence set forth in SEQ ID NO: 33 or a conservative modification thereof; or
(k) a heavy chain variable region CDR3 comprising the amino acid sequence set forth in SEQ ID NO: 38 or a conservative modification thereof; and a light chain variable region CDR3 comprising the amino acid sequence set forth in SEQ ID NO: 41 or a conservative modification thereof.
A19. The foregoing antibody or antigen-binding fragment thereof of A18, wherein the heavy chain variable region and light chain variable region CDR2 domains are selected from:
(a) a heavy chain variable region CDR2 comprising the amino acid sequence set forth in SEQ ID NO: 4 or a conservative modification thereof; and a light chain variable region CDR2 comprising the amino acid sequence set forth in SEQ ID NO: 7 or a conservative modification thereof;
(b) a heavy chain variable region CDR2 comprising the amino acid sequence set forth in SEQ ID NO: 29 or a conservative modification thereof; and a light chain variable region CDR2 comprising the amino acid sequence set forth in SEQ ID NO: 32 or a conservative modification thereof; or
(c) a heavy chain variable region CDR2 comprising the amino acid sequence set forth in SEQ ID NO: 37 or a conservative modification thereof; and a light chain variable region CDR2 comprising the amino acid sequence set forth in SEQ ID NO: 40 or a conservative modification thereof.
A20. The foregoing antibody or antigen-binding fragment thereof of A18 or A19, wherein the heavy chain variable region and light chain variable region CDR1 domains are selected from the group consisting of:
(a) a heavy chain variable region CDR1 comprising the amino acid sequence set forth in SEQ ID NO: 3 or a conservative modification thereof; and a light chain variable region CDR1 comprising the amino acid sequence set forth in SEQ ID NO: 6 or a conservative modification thereof;
(b) a heavy chain variable region CDR1 comprising the amino acid sequence set forth in SEQ ID NO: 3 or a conservative modification thereof; and a light chain variable region CDR1 comprising the amino acid sequence set forth in SEQ ID NO: 13 or a conservative modification thereof;
(c) a heavy chain variable region CDR1 comprising the amino acid sequence set forth in SEQ ID NO: 3 or a conservative modification thereof; and a light chain variable region CDR1 comprising the amino acid sequence set forth in SEQ ID NO: 18 or a conservative modification thereof;
(d) a heavy chain variable region CDR1 comprising the amino acid sequence set forth in SEQ ID NO: 3 or a conservative modification thereof; and a light chain variable region CDR1 comprising the amino acid sequence set forth in SEQ ID NO: 31 or a conservative modification thereof; or
(e) a heavy chain variable region CDR1 comprising the amino acid sequence set forth in SEQ ID NO: 36 or a conservative modification thereof; and a light chain variable region CDR1 comprising the amino acid sequence set forth in SEQ ID NO: 39 or a conservative modification thereof.
A21. The foregoing antibody or antigen-binding fragment thereof of any one of A18-A20, wherein one or more of the CDR sequences have up to about 5 amino acid substitutions.
A22. The foregoing antibody or antigen-binding fragment thereof of any one of A18-A21, wherein one or more of the CDR sequences have up to about 3 amino acid substitutions.
A23. The foregoing antibody or an antigen-binding fragment thereof of any one of A1-A8, comprising:
(a) a heavy chain variable region CDR1 comprising the amino acid sequence set forth in SEQ ID NO: 3; a heavy chain variable region CDR2 comprising the amino acid sequence set forth in SEQ ID NO: 4; and a heavy chain variable region CDR3 comprising the amino acid sequence set forth in SEQ ID NO: 5;
(b) a heavy chain variable region CDR1 comprising the amino acid sequence set forth in SEQ ID NO: 3; a heavy chain variable region CDR2 comprising the amino acid sequence set forth in SEQ ID NO: 29; and a heavy chain variable region CDR3 comprising the amino acid sequence set forth in SEQ ID NO: 30; or
(c) a heavy chain variable region CDR1 comprising the amino acid sequence set forth in SEQ ID NO: 36; a heavy chain variable region CDR2 comprising the amino acid sequence set forth in SEQ ID NO: 37; and a heavy chain variable region CDR3 comprising the amino acid sequence set forth in SEQ ID NO: 38.
A24. The foregoing antibody or antigen-binding fragment thereof of any one of A1-A8, comprising:
(a) a light chain variable region CDR1 comprising the amino acid sequence set forth in SEQ ID NO: 6; a light chain variable region CDR2 comprising the amino acid sequence set forth in SEQ ID NO: 7; and a light chain variable region CDR3 comprising the amino acid sequence set forth in SEQ ID NO: 8;
(b) a light chain variable region CDR1 comprising the amino acid sequence set forth in SEQ ID NO: 6; a light chain variable region CDR2 comprising the amino acid sequence set forth in SEQ ID NO: 7; and a light chain variable region CDR3 comprising the amino acid sequence set forth in SEQ ID NO: 11;
(c) a light chain variable region CDR1 comprising the amino acid sequence set forth in SEQ ID NO: 13; a light chain variable region CDR2 comprising the amino acid sequence set forth in SEQ ID NO: 7; and a light chain variable region CDR3 comprising the amino acid sequence set forth in SEQ ID NO: 14;
(d) a light chain variable region CDR1 comprising the amino acid sequence set forth in SEQ ID NO: 6; a light chain variable region CDR2 comprising the amino acid sequence set forth in SEQ ID NO: 7; and a light chain variable region CDR3 comprising the amino acid sequence set forth in SEQ ID NO: 16;
(e) a light chain variable region CDR1 comprising the amino acid sequence set forth in SEQ ID NO: 18; a light chain variable region CDR2 comprising the amino acid sequence set forth in SEQ ID NO: 7; and a light chain variable region CDR3 comprising the amino acid sequence set forth in SEQ ID NO: 19;
(f) a light chain variable region CDR1 comprising the amino acid sequence set forth in SEQ ID NO: 6; a light chain variable region CDR2 comprising the amino acid sequence set forth in SEQ ID NO: 7; and a light chain variable region CDR3 comprising the amino acid sequence set forth in SEQ ID NO: 21;
(g) a light chain variable region CDR1 comprising the amino acid sequence set forth in SEQ ID NO: 6; a light chain variable region CDR2 comprising the amino acid sequence set forth in SEQ ID NO: 7; and a light chain variable region CDR3 comprising the amino acid sequence set forth in SEQ ID NO: 23;
(h) a light chain variable region CDR1 comprising the amino acid sequence set forth in SEQ ID NO: 6; a light chain variable region CDR2 comprising the amino acid sequence set forth in SEQ ID NO: 7; and a light chain variable region CDR3 comprising the amino acid sequence set forth in SEQ ID NO: 25;
(i) a light chain variable region CDR1 comprising the amino acid sequence set forth in SEQ ID NO: 6; a light chain variable region CDR2 comprising the amino acid sequence set forth in SEQ ID NO: 7; and a light chain variable region CDR3 comprising the amino acid sequence set forth in SEQ ID NO: 27;
(j) a light chain variable region CDR1 comprising the amino acid sequence set forth in SEQ ID NO: 31; a light chain variable region CDR2 comprising the amino acid sequence set forth in SEQ ID NO: 32; and a light chain variable region CDR3 comprising the amino acid sequence set forth in SEQ ID NO: 33; or
(k) a light chain variable region CDR1 comprising the amino acid sequence set forth in SEQ ID NO: 39; a light chain variable region CDR2 comprising the amino acid sequence set forth in SEQ ID NO: 40; and a light chain variable region CDR3 comprising the amino acid sequence set forth in SEQ ID NO: 41.
A25. The foregoing antibody or antigen-binding fragment thereof of any one of A1-A8, wherein the heavy chain variable region comprises a CDR1 comprising the amino acid sequence set forth in SEQ ID NO: 3, a CDR2 comprising the amino acid sequence set forth in SEQ ID NO: 4, and a CDR3 comprising the amino acid sequence set forth in SEQ ID NO: 5; and the light chain variable region comprises a CDR1 comprising the amino acid sequence set forth in SEQ ID NO: 6, a CDR2 comprising the amino acid sequence set forth in SEQ ID NO: 7, and a CDR3 comprising the amino acid sequence set forth in SEQ ID NO: 8;
(b) the heavy chain variable region comprises a CDR1 comprising the amino acid sequence set forth in SEQ ID NO: 3, a CDR2 comprising the amino acid sequence set forth in SEQ ID NO: 4, and a CDR3 comprising the amino acid sequence set forth in SEQ ID NO: 5; and the light chain variable region comprises a CDR1 comprising the amino acid sequence set forth in SEQ ID NO: 6, a CDR2 comprising the amino acid sequence set forth in SEQ ID NO: 7, and a CDR3 comprising the amino acid sequence set forth in SEQ ID NO: 11;
(c) the heavy chain variable region comprises a CDR1 comprising the amino acid sequence set forth in SEQ ID NO: 3, a CDR2 comprising the amino acid sequence set forth in SEQ ID NO: 4, and a CDR3 comprising the amino acid sequence set forth in SEQ ID NO: 5; and the light chain variable region comprises a CDR1 comprising the amino acid sequence set forth in SEQ ID NO: 13, a CDR2 comprising the amino acid sequence set forth in SEQ ID NO: 7, and a CDR3 comprising the amino acid sequence set forth in SEQ ID NO: 14;
(d) the heavy chain variable region comprises a CDR1 comprising the amino acid sequence set forth in SEQ ID NO: 3, a CDR2 comprising the amino acid sequence set forth in SEQ ID NO: 4, and a CDR3 comprising the amino acid sequence set forth in SEQ ID NO: 5; and the light chain variable region comprises a CDR1 comprising the amino acid sequence set forth in SEQ ID NO: 6, a CDR2 comprising the amino acid sequence set forth in SEQ ID NO: 7, and a CDR3 comprising the amino acid sequence set forth in SEQ ID NO: 16;
(e) the heavy chain variable region comprises a CDR1 comprising the amino acid sequence set forth in SEQ ID NO: 3, a CDR2 comprising the amino acid sequence set forth in SEQ ID NO: 4, and a CDR3 comprising the amino acid sequence set forth in SEQ ID NO: 5; and the light chain variable region comprises a CDR1 comprising the amino acid sequence set forth in SEQ ID NO: 18, a CDR2 comprising the amino acid sequence set forth in SEQ ID NO: 7, and a CDR3 comprising the amino acid sequence set forth in SEQ ID NO: 19;
(f) the heavy chain variable region comprises a CDR1 comprising the amino acid sequence set forth in SEQ ID NO: 3, a CDR2 comprising the amino acid sequence set forth in SEQ ID NO: 4, and a CDR3 comprising the amino acid sequence set forth in SEQ ID NO: 5; and the light chain variable region comprises a CDR1 comprising the amino acid sequence set forth in SEQ ID NO: 6, a CDR2 comprising the amino acid sequence set forth in SEQ ID NO: 7, and a CDR3 comprising the amino acid sequence set forth in SEQ ID NO: 21;
(g) the heavy chain variable region comprises a CDR1 comprising the amino acid sequence set forth in SEQ ID NO: 3, a CDR2 comprising the amino acid sequence set forth in SEQ ID NO: 4, and a CDR3 comprising the amino acid sequence set forth in SEQ ID NO: 5; and the light chain variable region comprises a CDR1 comprising the amino acid sequence set forth in SEQ ID NO: 6, a CDR2 comprising the amino acid sequence set forth in SEQ ID NO: 7, and a CDR3 comprising the amino acid sequence set forth in SEQ ID NO: 23;
(h) the heavy chain variable region comprises a CDR1 comprising the amino acid sequence set forth in SEQ ID NO: 3, a CDR2 comprising the amino acid sequence set forth in SEQ ID NO: 4, and a CDR3 comprising the amino acid sequence set forth in SEQ ID NO: 5; and the light chain variable region comprises a CDR1 comprising the amino acid sequence set forth in SEQ ID NO: 6, a CDR2 comprising the amino acid sequence set forth in SEQ ID NO: 7, and a CDR3 comprising the amino acid sequence set forth in SEQ ID NO: 25;
(i) the heavy chain variable region comprises a CDR1 comprising the amino acid sequence set forth in SEQ ID NO: 3, a CDR2 comprising the amino acid sequence set forth in SEQ ID NO: 4, and a CDR3 comprising the amino acid sequence set forth in SEQ ID NO: 5; and the light chain variable region comprises a CDR1 comprising the amino acid sequence set forth in SEQ ID NO: 6, a CDR2 comprising the amino acid sequence set forth in SEQ ID NO: 7, and a CDR3 comprising the amino acid sequence set forth in SEQ ID NO: 27;
(j) the heavy chain variable region comprises a CDR1 comprising the amino acid sequence set forth in SEQ ID NO: 3, a CDR2 comprising the amino acid sequence set forth in SEQ ID NO: 29, and a CDR3 comprising the amino acid sequence set forth in SEQ ID NO: 30; and the light chain variable region comprises a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33; or
(k) the heavy chain variable region comprises a CDR1 comprising the amino acid sequence set forth in SEQ ID NO: 36, a CDR2 comprising the amino acid sequence set forth in SEQ ID NO: 37, and a CDR3 comprising the amino acid sequence set forth in SEQ ID NO: 38; and the light chain variable region comprises a CDR1 comprising the amino acid sequence set forth in SEQ ID NO: 39, a CDR2 comprising the amino acid sequence set forth in SEQ ID NO: 40, and a CDR3 comprising the amino acid sequence set forth in SEQ ID NO: 41.
A26. The foregoing antibody or antigen-binding fragment thereof of A25, wherein the heavy chain variable region comprises a CDR1 comprises the amino acid sequence set forth in SEQ ID NO: 3, a CDR2 comprises the amino acid sequence set forth in SEQ ID NO: 4, and a CDR3 comprises the amino acid sequence set forth in SEQ ID NO: 5; the light chain variable region comprises a CDR1 comprises the amino acid sequence set forth in SEQ ID NO: 6, a CDR2 comprises the amino acid sequence set forth in SEQ ID NO: 7, and a CDR3 comprises the amino acid sequence set forth in SEQ ID NO: 8.
A27. The foregoing antibody or antigen-binding fragment thereof of any one of A1-A26, wherein the antibody comprises a comprises a heavy chain constant region and/or a light chain constant region.
A28. The foregoing antibody or antigen-binding fragment thereof of A27, wherein:
(a) the heavy chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 50; and/or
(b) the light chain constant region comprises an amino acid sequence that is at least about 80%, at least about 85%, at least about 90%, at least about 95%, at least about 96%, at least about 97%, at least about 98%, at least about 99% at least about 100% homologous or identical to the amino acid sequence set forth in SEQ ID NO: 51.
A29. The antibody or antigen-binding fragment thereof of A27 or A28, wherein:
(a) the heavy chain constant region comprises the amino acid sequence set forth in SEQ ID NO: 50; and/or
(b) the light chain constant region comprises the amino acid sequence set forth in SEQ ID NO: 51.
A30. The foregoing antibody or antigen-binding fragment thereof of any one of A1-A29, wherein the antibody comprises a human variable region framework region.
A31. The foregoing antibody or antigen-binding fragment thereof of any one of A1-A30, which is a fully human or an antigen-binding fragment thereof.
A32. The foregoing antibody or antigen-binding fragment thereof of any one of A1-A31, which is a chimeric antibody or an antigen-binding fragment thereof.
A33. The foregoing antibody or antigen-binding fragment thereof of any one of A1-A32, which is a humanized antibody or an antigen-binding fragment thereof.
A34. The foregoing antibody or antigen-binding fragment thereof of any one of A1-A33, wherein the antigen-binding fragment is a Fab, Fab′, F(ab′)₂, variable fragment (Fv), or single chain variable region (scFv).
A35. The foregoing antibody or antigen-binding fragment thereof of A34, wherein the antigen antigen-binding fragment is an scFv.
B1. In certain non-limiting embodiments, the presently disclosed subject matter provides a composition comprising the antibody or antigen-binding fragment thereof of any one of A1-A35.
B2. The foregoing composition of B1, which is a pharmaceutical composition that further comprises a pharmaceutically acceptable carrier.
C1. In certain non-limiting embodiments, the presently disclosed subject matter provides an immunoconjugate comprising the antibody or antigen-binding fragment thereof of any one of A1-A35, linked to a therapeutic agent.
C2. The foregoing immunoconjugate of C1, wherein the therapeutic agent is a drug, a cytotoxin, or a radioactive isotope.
D1. In certain non-limiting embodiments, the presently disclosed subject matter provides a composition comprising the immunoconjugate of C1 or C2.
D2. The foregoing composition of D1, which is a pharmaceutical composition that further comprises a pharmaceutically acceptable carrier.
E1. In certain non-limiting embodiments, the presently disclosed subject matter provides a multi-specific molecule comprising the antibody or antigen-binding fragment thereof of any one of A1-A35, linked to one or more functional moieties.
E2. The foregoing multi-specific molecule of E1, wherein the one or more functional moieties have a different binding specificity than the antibody or antigen binding fragment thereof.
F1. In certain non-limiting embodiments, the presently disclosed subject matter provides a composition comprising the multi-specific molecule of E1 or E2.
F2. The foregoing composition of F1, which is a pharmaceutical composition that further comprises a pharmaceutically acceptable carrier.
G1. In certain non-limiting embodiments, the presently disclosed subject matter provides a nucleic acid that encodes an antibody or antigen-binding fragment thereof of any one of A1-A35.
G2. In certain non-limiting embodiments, the presently disclosed subject matter provides a nucleic acid that encodes a heavy chain variable region of an antibody or antigen-binding fragment thereof of any one of A1-A35.
G3. The foregoing nucleic acid of G2, comprising a nucleotide sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the nucleotide sequence set forth in SEQ ID NO: 52, SEQ ID NO: 54, or SEQ ID NO: 56.
G4. The foregoing nucleic acid of G2 or G3, comprising the nucleotide sequence set forth in SEQ ID NO: 52, SEQ ID NO: 54, or SEQ ID NO: 56.
G5. In certain non-limiting embodiments, the presently disclosed subject matter provides a nucleic acid that encodes a light chain variable region of an antibody or antigen-binding fragment thereof of any one of A1-A35.
G6. The foregoing nucleic acid of G5, comprising a nucleotide sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the nucleotide sequence set forth in SEQ ID NO: 53, SEQ ID NO: 55, or SEQ ID NO: 57.
G7. The foregoing nucleic acid of G5 or G6, comprising the nucleotide sequence set forth in SEQ ID NO: 53, SEQ ID NO: 55, or SEQ ID NO: 57.
H1. In certain non-limiting embodiments, the presently disclosed subject matter provides a vector comprising the nucleic acid of any one of G1-G7.
H2. In certain non-limiting embodiments, the presently disclosed subject matter provides a host cell comprising the vector of H1.
H3. In certain non-limiting embodiments, the presently disclosed subject matter provides a lipid nanoparticle comprising the nucleic acid of any one of G1-G7.
I1. In certain non-limiting embodiments, the presently disclosed subject matter provides a method for detecting HPV in a whole cell, a tissue, or a blood sample, comprising:
(a) contacting a cell, tissue or blood sample with the antibody or antigen-binding fragment thereof of any one of A1-A35, wherein the antibody or antigen-binding fragment thereof comprises a detectable label; and
(b) determining the amount of the labeled antibody or antigen-binding fragment thereof bound to the cell, tissue or blood sample by measuring the amount of detectable label associated with said cell or tissue, wherein the amount of bound antibody or antigen-binding fragment thereof indicates the amount of HPV in the cell, tissue or blood sample.
J1. In certain non-limiting embodiments, the presently disclosed subject matter provides an antigen-recognizing receptor comprising an extracellular antigen-binding domain that binds to an HPV epitope bound to a human major histocompatibility complex (MHC) molecule, a transmembrane domain, and an intracellular signaling domain.
J2. The foregoing antigen-recognizing receptor of J1, wherein the extracellular antigen-binding domain comprises the antigen-binding fragment of any one of A1-A35.
J3. The foregoing antigen-recognizing receptor of J1 or J2, wherein the antigen-recognizing receptor is a chimeric antigen receptor (CAR).
J4. The foregoing antigen-recognizing receptor of any one of J1-J3, wherein the intracellular signaling domain comprises a CD3ζ polypeptide.
J5. The foregoing antigen-recognizing receptor of any one of J1-J4, wherein the intracellular signaling domain further comprises at least one co-stimulatory signaling region.
J6. The foregoing antigen-recognizing receptor of J5, wherein the at least one co-stimulatory signaling region comprises an intracellular signaling domain of a co-stimulatory molecule.
J7. The foregoing antigen-recognizing receptor of J6, wherein the co-stimulatory molecule is selected from the group consisting of CD28, 4-1BB, OX40, ICOS, DAP-10, CD27, CD40, NKGD2, CD2, FN14, HVEM, LTBR, CD28H, TNFR1, TNFR2, BAFF-R, BCMA, TACI, TROY, RANK, CD40, CD27, CD30, EDAR, XEDAR, GITR, DR6, NGFR, and combinations thereof.
J8. The foregoing antigen-recognizing receptor of J7, wherein the co-stimulatory molecule is CD28 or 4-1BB.
J9. The foregoing antigen-recognizing receptor of J1 or J2, wherein the antigen-recognizing receptor is a TCR like fusion molecule (HIT) or a T cell receptor fusion construct (TRuC).
K1. In certain non-limiting embodiments, the presently disclosed subject matter provides a nucleic acid that encodes an antigen-recognizing receptor of any one of J1-J9.
K2. In certain non-limiting embodiments, the presently disclosed subject matter provides a vector comprising the nucleic acid of K1.
K3. In certain non-limiting embodiments, the presently disclosed subject matter provides a lipid nanoparticle comprising the nucleic acid of K1.
L1. In certain non-limiting embodiments, the presently disclosed subject matter provides a cell comprising the antigen-recognizing receptor of any one of J1-J9.
L2. In certain non-limiting embodiments, the presently disclosed subject matter provides a composition comprising the cell of L1.
M1. In certain non-limiting embodiments, the presently disclosed subject matter provides a method of treating or ameliorating a disease or disorder in a subject, comprising administering to the subject the antibody or antigen-binding fragment thereof of any one of A1-A35, the immunoconjugate of C1 or C2, the multi-specific molecule of E1 or E2, the cell of L1, or the composition of any one of B1, B2, D1, D2, F1, F2, or L2.
M2. The foregoing method of M1, wherein the disease or disorder is a tumor.
M3. In certain non-limiting embodiments, the presently disclosed subject matter provides a method of reducing tumor burden in a subject, comprising administering to the subject an antibody or antigen-binding fragment thereof of any one of A1-A35, the immunoconjugate of C1 or C2, the multi-specific molecule of E1 or E2, the cell of L1, or the composition of any one of B1, B2, D1, D2, F1, F2, or L2.
M4. The foregoing method of M3, wherein the method reduces the number of the tumor cells, reduces the tumor size, and/or eradicates the tumor in the subject.
M5. In certain non-limiting embodiments, the presently disclosed subject matter provides a method of treating and/or preventing a tumor in a subject, comprising administering to the subject an antibody or antigen-binding fragment thereof of any one of A1-A35, the immunoconjugate of C1 or C2, the multi-specific molecule of E1 or E2, the cell of L1, or the composition of any one of B1, B2, D1, D2, F1, F2, or L2.
M6. In certain non-limiting embodiments, the presently disclosed subject matter provides a method of increasing or lengthening survival of a subject having a tumor, comprising administering to the subject an antibody or antigen-binding fragment thereof of any one of A1-A35, the immunoconjugate of C1 or C2, the multi-specific molecule of E1 or E2, the cell of L1, or the composition of any one of B1, B2, D1, D2, F1, F2, or L2.
M7. The foregoing method of M6, wherein the method reduces or eradicates tumor burden in the subject.
M8. The foregoing method of any one of M1-M7, wherein the disease or disorder or tumor is a cancer.
M9. The foregoing method of any one of M1-M8, wherein the disease or disorder or tumor is an HPV-associated tumor.
M10. The foregoing method of any one of M1-M9, wherein the disease or disorder or tumor is selected from the group consisting of ovarian cancer, cervical cancer, anal cancer, vaginal cancer, vulvar cancer, penile cancer, head and neck cancer, oropharynx cancer, oropharyngeal squamous cell carcinoma (SCC), anal & rectal SCC, vulvar SCC, vaginal SCC, cervical carcinoma, and penile SCC.
M11. The foregoing method of M10, wherein the cancer is ovarian cancer.
M12. The foregoing method of any one of M1-M11, wherein the subject is a human.
N1. In certain non-limiting embodiments, the presently disclosed subject matter provides a kit for treating or ameliorating a disease or disorder in a subject, reducing tumor burden in a subject, treating and/or preventing a tumor in a subject, and/or increasing or lengthening survival of a subject having a tumor, comprising the antibody or antigen-binding fragment thereof of any one of A1-A35, the immunoconjugate of C1 or C2, the multi-specific molecule of E1 or E2, the cell of L1, or the composition of any one of B1, B2, D1, D2, F1, F2, or L2.
N2. The foregoing kit of N1, wherein the kit further comprises written instructions for using the antibody or antigen-binding fragment thereof, immunoconjugate, multi-specific molecule, or composition for treating or ameliorating a disease or disorder in a subject, treating or ameliorating a disease or disorder in a subject, reducing tumor burden in a subject, treating and/or preventing a tumor in a subject, and/or increasing or lengthening survival of a subject having a tumor.

What is claimed is:

1. An antibody or an antigen-binding fragment thereof that binds to an HPV epitope bound to a human major histocompatibility complex (MHC) molecule, wherein the antibody or antigen-binding fragment thereof comprises a heavy chain variable region and a light chain variable region, wherein
(a) the heavy chain variable region comprises a CDR1 comprising the amino acid sequence set forth in SEQ ID NO: 3, a CDR2 comprising the amino acid sequence set forth in SEQ ID NO: 4, and a CDR3 comprising the amino acid sequence set forth in SEQ ID NO: 5; and the light chain variable region comprises a CDR1 comprising the amino acid sequence set forth in SEQ ID NO: 6, a CDR2 comprising the amino acid sequence set forth in SEQ ID NO: 7, and a CDR3 comprising the amino acid sequence set forth in SEQ ID NO: 8;
(b) the heavy chain variable region comprises a CDR1 comprising the amino acid sequence set forth in SEQ ID NO: 3, a CDR2 comprising the amino acid sequence set forth in SEQ ID NO: 4, and a CDR3 comprising the amino acid sequence set forth in SEQ ID NO: 5; and the light chain variable region comprises a CDR1 comprising the amino acid sequence set forth in SEQ ID NO: 6, a CDR2 comprising the amino acid sequence set forth in SEQ ID NO: 7, and a CDR3 comprising the amino acid sequence set forth in SEQ ID NO: 11;
(c) the heavy chain variable region comprises a CDR1 comprising the amino acid sequence set forth in SEQ ID NO: 3, a CDR2 comprising the amino acid sequence set forth in SEQ ID NO: 4, and a CDR3 comprising the amino acid sequence set forth in SEQ ID NO: 5; and the light chain variable region comprises a CDR1 comprising the amino acid sequence set forth in SEQ ID NO: 13, a CDR2 comprising the amino acid sequence set forth in SEQ ID NO: 7, and a CDR3 comprising the amino acid sequence set forth in SEQ ID NO: 14;
(d) the heavy chain variable region comprises a CDR1 comprising the amino acid sequence set forth in SEQ ID NO: 3, a CDR2 comprising the amino acid sequence set forth in SEQ ID NO: 4, and a CDR3 comprising the amino acid sequence set forth in SEQ ID NO: 5; and the light chain variable region comprises a CDR1 comprising the amino acid sequence set forth in SEQ ID NO: 6, a CDR2 comprising the amino acid sequence set forth in SEQ ID NO: 7, and a CDR3 comprising the amino acid sequence set forth in SEQ ID NO: 16;
(e) the heavy chain variable region comprises a CDR1 comprising the amino acid sequence set forth in SEQ ID NO: 3, a CDR2 comprising the amino acid sequence set forth in SEQ ID NO: 4, and a CDR3 comprising the amino acid sequence set forth in SEQ ID NO: 5; and the light chain variable region comprises a CDR1 comprising the amino acid sequence set forth in SEQ ID NO: 18, a CDR2 comprising the amino acid sequence set forth in SEQ ID NO: 7, and a CDR3 comprising the amino acid sequence set forth in SEQ ID NO: 19;
(f) the heavy chain variable region comprises a CDR1 comprising the amino acid sequence set forth in SEQ ID NO: 3, a CDR2 comprising the amino acid sequence set forth in SEQ ID NO: 4, and a CDR3 comprising the amino acid sequence set forth in SEQ ID NO: 5; and the light chain variable region comprises a CDR1 comprising the amino acid sequence set forth in SEQ ID NO: 6, a CDR2 comprising the amino acid sequence set forth in SEQ ID NO: 7, and a CDR3 comprising the amino acid sequence set forth in SEQ ID NO: 21;
(g) the heavy chain variable region comprises a CDR1 comprising the amino acid sequence set forth in SEQ ID NO: 3, a CDR2 comprising the amino acid sequence set forth in SEQ ID NO: 4, and a CDR3 comprising the amino acid sequence set forth in SEQ ID NO: 5; and the light chain variable region comprises a CDR1 comprising the amino acid sequence set forth in SEQ ID NO: 6, a CDR2 comprising the amino acid sequence set forth in SEQ ID NO: 7, and a CDR3 comprising the amino acid sequence set forth in SEQ ID NO: 23;
(h) the heavy chain variable region comprises a CDR1 comprising the amino acid sequence set forth in SEQ ID NO: 3, a CDR2 comprising the amino acid sequence set forth in SEQ ID NO: 4, and a CDR3 comprising the amino acid sequence set forth in SEQ ID NO: 5; and the light chain variable region comprises a CDR1 comprising the amino acid sequence set forth in SEQ ID NO: 6, a CDR2 comprising the amino acid sequence set forth in SEQ ID NO: 7, and a CDR3 comprising the amino acid sequence set forth in SEQ ID NO: 25;
(i) the heavy chain variable region comprises a CDR1 comprising the amino acid sequence set forth in SEQ ID NO: 3, a CDR2 comprising the amino acid sequence set forth in SEQ ID NO: 4, and a CDR3 comprising the amino acid sequence set forth in SEQ ID NO: 5; and the light chain variable region comprises a CDR1 comprising the amino acid sequence set forth in SEQ ID NO: 6, a CDR2 comprising the amino acid sequence set forth in SEQ ID NO: 7, and a CDR3 comprising the amino acid sequence set forth in SEQ ID NO: 27;
(j) the heavy chain variable region comprises a CDR1 comprising the amino acid sequence set forth in SEQ ID NO: 3, a CDR2 comprising the amino acid sequence set forth in SEQ ID NO:
29, and a CDR3 comprising the amino acid sequence set forth in SEQ ID NO: 30; and the light chain variable region comprises a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33; or
(k) the heavy chain variable region comprises a CDR1 comprising the amino acid sequence set forth in SEQ ID NO: 36, a CDR2 comprising the amino acid sequence set forth in SEQ ID NO: 37, and a CDR3 comprising the amino acid sequence set forth in SEQ ID NO: 38; and the light chain variable region comprises a CDR1 comprising the amino acid sequence set forth in SEQ ID NO: 39, a CDR2 comprising the amino acid sequence set forth in SEQ ID NO: 40, and a CDR3 comprising the amino acid sequence set forth in SEQ ID NO: 41.

2. The antibody or antigen-binding fragment thereof of claim 1, wherein the heavy chain variable region comprises a CDR1 comprises the amino acid sequence set forth in SEQ ID NO: 3, a CDR2 comprises the amino acid sequence set forth in SEQ ID NO: 4, and a CDR3 comprises the amino acid sequence set forth in SEQ ID NO: 5; the light chain variable region comprises a CDR1 comprises the amino acid sequence set forth in SEQ ID NO: 6, a CDR2 comprises the amino acid sequence set forth in SEQ ID NO: 7, and a CDR3 comprises the amino acid sequence set forth in SEQ ID NO: 8.

3. The antibody or antigen-binding fragment thereof of claim 1, wherein the heavy chain variable region and the light chain variable region are selected from the group consisting of:
(a) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 9, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 10;
(b) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 9, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 12;
(c) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 9, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 15;
(d) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 9, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 17;
(e) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 9, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 20;
(f) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 9, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 22;
(g) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 9, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 24;
(h) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 9, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 26;
(i) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 9, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 28;
(j) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 34, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 35; and
(k) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 42, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 43.

4. The antibody or antigen-binding fragment thereof of claim 1, wherein
(a) the heavy chain variable region comprises the amino acid sequence set forth in SEQ ID NO: 9, and the light chain variable region comprises the amino acid sequence set forth in SEQ ID NO: 10;
(b) the heavy chain variable region comprises the amino acid sequence set forth in SEQ ID NO: 9, and the light chain variable region comprises the amino acid sequence set forth in SEQ ID NO: 12;
(c) the heavy chain variable region comprises the amino acid sequence set forth in SEQ ID NO: 9, and the light chain variable region comprises the amino acid sequence set forth in SEQ ID NO: 15;
(d) the heavy chain variable region comprises the amino acid sequence set forth in SEQ ID NO: 9, and the light chain variable region comprises the amino acid sequence set forth in SEQ ID NO: 17;
(e) the heavy chain variable region comprises the amino acid sequence set forth in SEQ ID NO: 9, and the light chain variable region comprises the amino acid sequence set forth in SEQ ID NO: 20;
(f) the heavy chain variable region comprises the amino acid sequence set forth in SEQ ID NO: 9, and the light chain variable region comprises the amino acid sequence set forth in SEQ ID NO: 22;
(g) the heavy chain variable region comprises the amino acid sequence set forth in SEQ ID NO: 9, and the light chain variable region comprises the amino acid sequence set forth in SEQ ID NO: 24;
(h) the heavy chain variable region comprises the amino acid sequence set forth in SEQ ID NO: 9, and the light chain variable region comprises the amino acid sequence set forth in SEQ ID NO: 26;
(i) the heavy chain variable region comprises the amino acid sequence set forth in SEQ ID NO: 9, and the light chain variable region comprises the amino acid sequence set forth in SEQ ID NO: 28;
(j) the heavy chain variable region comprises the amino acid sequence set forth in SEQ ID NO: 34, and the light chain variable region comprises the amino acid sequence set forth in SEQ ID NO: 35; or
(k) the heavy chain variable region comprises the amino acid sequence set forth in SEQ ID NO: 42, and the light chain variable region comprises the amino acid sequence set forth in SEQ ID NO: 43.

5. The antibody or antigen-binding fragment thereof of claim 4, wherein the heavy chain variable region comprises the amino acid sequence set forth in SEQ ID NO: 9, and the light chain variable region comprises the amino acid sequence set forth in SEQ ID NO: 10.

6. The antibody or antigen-binding fragment thereof of claim 1, wherein the antibody comprises a comprises a heavy chain constant region and/or a light chain constant region.

7. The antibody or antigen-binding fragment thereof of claim 6, wherein:
(a) the heavy chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 50; and/or
(b) the light chain constant region comprises an amino acid sequence that is at least about 80%, at least about 85%, at least about 90%, at least about 95%, at least about 96%, at least about 97%, at least about 98%, at least about 99% at least about 100% homologous or identical to the amino acid sequence set forth in SEQ ID NO: 51.

8. The antibody or antigen-binding fragment thereof of claim 6, wherein:
(a) the heavy chain constant region comprises the amino acid sequence set forth in SEQ ID NO: 50; and/or
(b) the light chain constant region comprises the amino acid sequence set forth in SEQ ID NO: 51.

9. The antibody or antigen-binding fragment thereof of claim 1, wherein (a) the antibody or antigen-binding fragment thereof comprises a human variable region framework region; (b) the antibody or antigen-binding fragment thereof is a fully human or an antigen-binding fragment thereof; (c) the antibody or antigen-binding fragment thereof is a chimeric antibody or an antigen-binding fragment thereof; (d) the antibody or antigen-binding fragment thereof is a humanized antibody or an antigen-binding fragment thereof.

10. The antibody or antigen-binding fragment thereof of claim 1, wherein the antigen-binding fragment is a Fab, Fab′, F(ab′)₂, variable fragment (Fv), or single chain variable region (scFv).

11. A composition comprising the antibody or antigen-binding fragment thereof of claim 1.

12. An immunoconjugate comprising the antibody or antigen-binding fragment thereof of claim 1, linked to a therapeutic agent.

13. A multi-specific molecule comprising the antibody or antigen-binding fragment thereof of claim 1, linked to one or more functional moieties.

14. A nucleic acid that encodes an antibody or antigen-binding fragment thereof of claim 1.

15. A vector comprising the nucleic acid of any one of claim 14.

16. A host cell comprising the vector of claim 15.

17. A lipid nanoparticle comprising the nucleic acid of claim 17.

18. An antigen-recognizing receptor comprising an extracellular antigen-binding domain that binds to an HPV epitope bound to a human major histocompatibility complex (MHC) molecule, a transmembrane domain, and an intracellular signaling domain, wherein the extracellular antigen-binding domain comprises the antigen-binding fragment of claim 1.

19. A cell comprising the antigen-recognizing receptor of claim 18.

20. A method of (a) treating or ameliorating a disease or disorder in a subject, (b) reducing tumor burden in a subject, (c) increasing or lengthening survival of a subject having a tumor, the method comprising administering to the subject the antibody or antigen-binding fragment thereof of claim 1.

21. The method of claim 20, wherein the disease or disorder or tumor is an HPV-associated tumor.

22. The method of claim 20, wherein the disease or disorder or tumor is selected from the group consisting of ovarian cancer, cervical cancer, anal cancer, vaginal cancer, vulvar cancer, penile cancer, head and neck cancer, oropharynx cancer, oropharyngeal squamous cell carcinoma (SCC), anal & rectal SCC, vulvar SCC, vaginal SCC, cervical carcinoma, and penile SCC.

23. A kit for treating or ameliorating a disease or disorder in a subject, reducing tumor burden in a subject, treating and/or preventing a tumor in a subject, and/or increasing or lengthening survival of a subject having a tumor, comprising the antibody or antigen-binding fragment thereof of claim 1.
